(19) United States
(12) Patent Application Publication
(10) Pub. No.: US 2004/0018522 A1
(43) Pub. Date: Jan. 29, 2004

(54) Identification of dysregulated genes in patients with multiple sclerosis

(75) Inventors: Fernando Dangond, Newton, MA (US); Daehee Hwang, Seattle, WA (US)

Correspondence Address: FULBRIGHT & JAWORSKI L.L.P., Suite 2400, 600 CONGRESS AVENUE, AUSTIN, TX, 78701-3271, US

(73) Assignee: Brigham and Women's Hospital, Inc.

(21) Appl. No.: 10/430,762

(22) Filed: May 6, 2003

Related U.S. Application Data
(60) Provisional application No. 60/379,284, filed on May 9, 2002.

(51) Int. Cl. C12Q 1/68; G06F 19/00; G01N 33/48; G01N 33/50

(52) U.S. Cl. 435/6; 702/20

(57) ABSTRACT

The present invention identifies a number of gene markers whose expression is altered in multiple sclerosis (MS). These markers can be used to diagnose or predict MS in subjects, and can be used in the monitoring of therapies. In addition, these genes identify therapeutic targets, the modification of which may prevent MS development or progression.

### BACKGROUND OF THE INVENTION

The present application claims benefit of priority to U.S. Provisional Serial No. 60/379,284, filed May 9, 2003, the entire contents of which are hereby incorporated by reference.

1. Field of the Invention

The present invention relates generally to the fields of molecular biology, genomics, immunology and neurobiology. More particularly, it concerns the identification of specific genes that are dysregulated in patients afflicted with multiple sclerosis (MS). These genes can be used to identify subjects suffering from or at risk of MS, and can also provide targets for MS therapies.

2. Description of Related Art

Multiple sclerosis (MS) continues to be a serious health problem that afflicts hundreds of thousands each year in the US alone, and millions worldwide. One of the difficult aspects of dealing with MS is identifying patients early in the course of the disease. This is difficult not only because of the lack of a definitive biological test for MS, but because the symptoms may overlap with those of numerous other diseases.

The concordance rate of multiple sclerosis among monozygotic twins is 20-40%, while the risk of a non-twin sibling of an MS patient of developing MS is 2-4%. These facts highly suggest the presence of polygenic susceptibility (nonmendelian inheritance). Although no single gene is associated with all types of MS, several reports have revealed that some genes are associated with MS in certain populations. The well known HLA association with MS has been demonstrated in populations of northern European ancestry. In the Finnish population an association with the myclin basic protein gene has been reported (Tienari et al., 1992). In an European MS patient population, an association with a T cell differentiation-related antigen, CD45, has been demonstrated (Jacobsen et al., 2000).

Since the disease is polymorphic (i.e., not inherited in a classical mendelian pattern but clearly multiple genes are involved in leading to predisposition), recent genomic approaches have been implemented to elucidate multiple genes simultaneously that may be associated with the disease. A recent publication by Lock et al., 2002, demonstrates how gene expression profiling using DNA microarrays to examine MS brain tissues can help identify multiple single genes that are associated with the disease, and may therefore serve as targets of treatment. By altering the function of the product of some of these genes in the animal model of MS, experimental autoimmune encephalomyelitis (EAE), these authors confirmed that some genes found to be altered by DNA microarray screening indeed had an impact on the severity of the disease.

Another approach to identify potential single gene associations is to examine polymorphic gene variants or single nucleotide polymorphisms (SNPs) of candidate genes, or screen the entire genome to establish the SNPs that are associated with the disease. Multiple polymorphisms have been associated with MS, as follows: a) polymorphisms associated with MS disease susceptibility found in the following genes: SCA2 (Chataway et al., 1999), interferon α (Miterski et al., 1999), estrogen receptor (Niino et al., 2000), plasminogen activator inhibitor 1 (Luomala et al., 2000), tumor necrosis factor a (Femandez-Arquero et al., 1999; Lucotte et al., 2000), monocyte chemotactic protein 3 (Fiten et al., 1999), vitamin D receptor (Fukazawa et al., 1999), CTLA4 (Fukazawa et al., 1999), γ aminobutyric acid (Gade-Andavolu et al., 1998); b) polymorphisms associated with disease severity found in the following genes: interleukin 6 (Vandenbroeck et al., 2000), IgG Fe receptor (FCγ R) (Myhr et al., 1999), glutathione-S-transferase (Mann et al., 2000); c) polymorphisms associated with age of onset of MS found in the following genes: interleukin 4 (Vandenbroeck et al., 1997) and chemokine receptor CCR5 (Barcellos et al., 2000); and d) polymorphism associated with remyclination capacity: apolipoprotein E (Carlin et al., 2000). Other gene polymorphisms that have been associated with MS include intercellular adhesion molecule 1 (ICAM-1) (Mycko et al., 1998), the pro-inflammatory gene lymphotoxin (Mycko et al., 1998) and immunoglobulin heavy chain gene polymorphisms (Hashimoto et al., 1993; Walter et al., 1991).

Despite these individual associations, there has yet to be put forth a cohesive set of genes that, as a group, provide accurate diagnostic or prognostic information regarding MS. Thus, there remains a need for a genetic based test that identifies individuals having or at risk of developing MS.

### SUMMARY OF THE INVENTION

Thus, in accordance with the present invention, there is provided a method of predicting whether a subject is or will be afflicted with multiple sclerosis (MS) comprising (a) obtaining an mRNA-containing sample from said subject; (b) determining expression information for one or more genes from the set of highest discriminatory genes selected from the group consisting of phosphatidylinositol transfer protein, inducible nitric oxide synthase, CIC-1 (CLCN1) muscle chloride channel protein, placental bikunin (AMBP), receptor kinase ligand LERK-3/Ephrin-A3, GATA-4, thymopoietin, transcription factor E2f-2, S-adenosylmethionine synthetase, carcinoembryonic antigen, ret transforming gene, G protein-linked receptor (clone GPCR W), GTP-binding protein RALB, tyrosine kinase Syk, T cell leukemia LERK-2/Ephrin-B1, tyrosine kinase (ELK1) oncogene, transcription factor SL1, phospholipase c, gastricsin (progastricsin), and D13S824E locus; (c) comparing expression information for said selected genes with the expression information of the same genes in a subject not afflicted with MS; and (d) predicting whether said subject is or will be afflicted with MS. The sample may comprise peripheral blood-derived mononuclear cells. The method may further comprise determining expression information for one or more genes in Tables 1, 2, 3, 4, 5, 6, 7, 8, 9, 10, 11 and 12. The method may also further comprise obtaining expression information for each gene in step (b), or for one or more genes in Tables 1, 2, 3, 4, 5, 6, 7, 8, 9, 10, 11 and 12, from a subject not afflicted with MS. The expression information for more than one gene in the group above may be determined, such as expression information for at least 2, 3, 4, 5, 6, 7, 8, 9, 10, 11, 12, 13, 14, 15, 16, 17, 18, 19 or 20 genes.

Expression information may be determined by microarray analysis of mRNA transcripts, by northern blots, RNAse protection assays, and multiplex or real-time PCR of mRNA transcripts, or by immunohistochemistry, ELISA or western analysis. Microarray analysis may comprise use of oligonucleotides that hybridize to mRNA transcripts or cDNAs for the selected genes, and the oligonucleotides may be disposed or directly synthesized on the surface of a chip or wafer. The oligonucleotides may be about 10 to about 50 base pairs in length.

In another embodiment, there is provided a chip or wafer comprising a nucleic acid microarray, wherein said nucleic acids hybridize to target transcripts or cDNAs for phosphatidylinositol transfer protein, inducible nitric oxide synthase, CIC-1 (CLCN1) muscle chloride channel protein, placental bikunin (AMBP), receptor kinase ligand LERK-3/Ephrin-A3, GATA-4, thymopoietin, transcription factor E2f-2, S-adenosylmethionine synthetase, carcinoembryonic antigen, ret transforming gene, G protein-linked receptor (clone GPCR W), GTP-binding protein RALB, tyrosine kinase Syk, T cell leukemia LERK-2/Ephrin-B1, tyrosine kinase (ELK1) oncogene, transcription factor SL1, phospholipase c, gastricsin (progastricsin), and D13S824E locus. The chip may be comprised of polymers, plastics, resins, polysaccharides, silica or silica-based materials, fiberoptic materials, carbon, metals, inorganic glasses, or nitrocellulose. The nucleic acids may be partial or full length cDNAs, or oligonucleotides of about 10 to about 50 base pairs or less in length.

In yet another embodiment, there is provided a method for monitoring a therapy for multiple sclerosis comprising (a) obtaining an mRNA-containing sample from a subject receiving said therapy; (b) determining expression information for one or more genes comprising phosphatidylinositol transfer protein, inducible nitric oxide synthase, CIC-1 (CLCN1) muscle chloride channel protein, placental bikunin (AMBP), receptor kinase ligand LERK-3/Ephrin-A3, GATA-4, thymopoietin, and transcription factor E2f-2, S-adenosylmethionine synthetase, carcinoembryonic antigen, ret transforming gene, G protein-linked receptor (clone GPCR W), GTP-binding protein RALB, tyrosine kinase Syk, T cell leukemia LERK-2/Ephrin-B1, tyrosine kinase (ELK1) oncogene, transcription factor SL1, phospholipase c, gastricsin (progastricsin), and D13S824E locus; and (c) comparing expression information for said selected genes with the expression information of the same genes in an MS subject not receiving said therapy. The sample may be peripheral blood. The method may further comprise modifying said therapy based upon the altered expression of one or more of said selected genes. The method may further comprise making a prediction on the efficacy of treating the subject from which said sample was obtained. The expression information may be determined by microarray analysis of mRNA transcripts, by northern blots, RNAse protection assays, and multiplex or real-time PCR of mRNA transcripts, or by immunohistochemistry, ELISA or western analysis.

The microarray analysis may comprise use of oligonucleotides that hybridize to transcripts or cDNAs for the selected genes, and wherein the oligonucleotides are disposed or synthesized directly on the surface of a chip or wafer. The chip may be comprised of polymers, plastics, resins, polysaccharides, silica or silica-based materials, carbon, metals, inorganic glasses, or nitrocellulose. The oligonucleotides may be about 10 to about 50 base pairs or less in length. The MS subject not receiving said therapy may be the same subject prior to receiving said therapy. The method may further comprise determining expression information for said selected genes from said subject at multiple time points.

In still yet another embodiment, there is provided a method for determining the efficacy of a therapy for multiple sclerosis comprising (a) obtaining an mRNA-containing sample from a subject receiving said therapy; (b) determining expression information for one or more selected genes selected from the group consisting of skeletal muscle LIM-protein SLIM1 (also known as four and a half LIM domains 1 (FHL1)), R kappa B, 815A9.1 myosin heavy chain (MYH11), γ G2 psi from γ crystallin, thrombospondin 4 and KIAA0178 (OR Z97054); (c) comparing expression information for said one or more selected genes with the expression information of the same gene or genes in an MS subject not receiving said therapy; and (d) determining the efficacy of said therapy based on the ability of said therapy to alter the expression of said one or more genes. The method may further comprise determining expression information for one or more genes in Table 14 or Table 15.

In a further embodiment, there is provided a method for treating multiple sclerosis (MS) comprising administering to a subject with MS a drug that causes an increase in the level of a gene product selected from the group consisting of those genes indicated by a minus (−) sign in Tables 1-12. In still yet a further embodiment, there is provided a method for treating multiple sclerosis (MS) comprising administering to a subject with MS a drug that causes a decrease in the level of a gene product selected from the group consisting of those genes indicated by a plus (+) sign in Tables 1-12 and 16.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Estimation of false positive error based on H₀ and H₁ distributions.

FIG. 2—FDA projections of peripheral blood samples from four classes. ALS=amyotrophic lateral sclerosis; Healthy=healthy donor; MS=multiple sclerosis; MSA=multiple sclerosis patients on Avonex.

FIG. 3—Eight preferred genes used to determine whether a given blood sample originated from an MS patient. MS vs. ALS and healthy donors were compared using 7 MS (a heterogeneous population), 8 ALS, and 7 healthy donor samples. This list was then compared to the lists from Tables 7-9, in which only 5 samples are included in each group (the 5 MS samples in Tables 7-9 are homogeneous, all with classical relapsing-remitting MS on no treatment). Eight genes were selected based on false positive error probability (only discriminatory genes whose false positive errors are less than 0.001 and are shared in these lists are selected). Autoscaled expressions were calculated by standardizing each gene's expression into zero mean and unit variance (variance=1).

FIGS. 4A-D—In vitro and in vivo validation of MS PBMC targets. FIG. 4A. Quantitative reverse transcriptase-polymerase chain reaction (QRT-PCR) fold change results of selected genes, including the E2F1 transcription-dependent E2F2 and the myelin gene MOBP, in a set of MS PBMC-derived RNAs unrelated to those used for microarray analysis, as compared to healthy donors. FIG. 4B. Demonstration that E2f1 deficient mice (●) have lower disability scores (less disabling form of the disease) during myelin oligodendrocyte glycoprotein (MOG)-induced EAE (P<0.05 by Fisher's protected least significant difference (PLSD) test in days denoted by thick lines under the graph) as compared to wild type (WT) EAE mice (□) and C57BL/6 EAE mice (▴) (n=7 each group); the day of disease onset was not significantly altered. FIG. 4C. Propidium iodide-fluorescence activated cell sorter (PI-FACS) cell cycle analysis of splenocytes isolated from all E2f1 deficient (gray bars), WT EAE (black bars) and C57BL/6 (white bars) mice in the study. Splenocytes from E2f1 deficient mice with EAE exhibit decreased entry into S phase, as compared to splenocytes from WT EAE mice (* P<0.05 by Fisher's PLSD).

### DESCRIPTION OF ILLUSTRATIVE EMBODIMENTS

In autoimmune diseases, activated T cells, B cells and monocytes are hypothesized to clonally expand (i.e., proliferate into multiple daughter cells) and lead to tissue destruction, via infiltration of target tissues with direct cytotoxicity and/or release of harming soluble factors or antibodies. MS is widely considered an autoimmune disease, but there is significant controversy about the key molecules that participate in such process. Further, it is known that various autoimmune disorders, such as systemic lupus erythematosus (SLE), rheumatoid arthritis and MS, do not share all aspects of autoimmunity at the molecular level. This makes sense since it is believed that these different mechanisms are responsible for the target tissue specificity, the difference in biomarkers required to confirm the diagnosis, and the different types of lesions seen.

Therefore, the inventors used DNA microarrays to identify the gene signature of peripheral blood mononuclear cells in multiple sclerosis, revealing multiple new genes that have not been previously considered as biomarkers for this disease. In a comparison of healthy donors to either MS (an autoimmune disease) or ALS (not autoimmune), massive changes were found in gene expression, as would be expected, only in MS peripheral blood. Thus, the inventors have used ALS as a negative disease control to dissect out the autoimmune disease-related biomarkers. In addition, the inventors encountered gene expression abnormalities on a series of pathways, some of which have been previously implicated in autoimmunity, leading further credence to the accuracy of their method in establishing novel and specific biomarkers.

I. Multiple Sclerosis

Multiple Sclerosis (MS) is one of the most common diseases of the central nervous system (brain and spinal cord). It is an inflammatory condition caused by demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord, hence the term “multiple” sclerosis, literally meaning many scars.

Currently, there is no single laboratory test, symptom, or physical finding that provides a conclusive diagnosis of MS. To complicate matters, symptoms of MS can easily be confused with a wide variety of other diseases such as acute disseminated encephalomyclitis, Lyme disease, HIV-associated myelopathy, HTLV-1-associated myelopathy, neurosyphilis, progressive multifocal leukoencephalopathy, systemic lupus erythematosus, polyarteritis nodosa, Sjögren's syndrome, Behcet's disease, sarcoidosis, paraneoplastic syndromes, subacute combined degeneration of cord, subacute myelo-optic neuropathy, adrenomyeloneuropathy, spinocerebellar syndromes, hereditary spastic paraparesis/primary lateral sclerosis, strokes, tumors, arterioyenous malformations, arachnoid cysts, Amold-Chiari malformations, and cervical spondylosis. Consequently, the diagnosis of MS must be made by a process that demonstrates findings that are consistent with MS, and also rules out other causes.

Generally, the diagnosis of MS relies on two criteria. First, there must have been two attacks at least one month apart. An attack, also known as an exacerbation, flare, or relapse, is a sudden appearance of or worsening of an MS symptom or symptoms which lasts at least 24 hours. Second, there must be more than one anatomical area of damage to central nervous system myclin sheath. Damage to sheath must have occurred at more than one point in time and not have been caused by any other disease that can cause demyelination or similar neurologic symptoms. MRI (magnetic resonance imaging) currently is the preferred method of imaging the brain to detect the presence of plaques or scarring caused by MS.

The diagnosis of MS cannot be made, however, solely on the basis of MRI. Other diseases can cause comparable lesions in the brain that resemble those caused by MS. Furthermore, the appearance of brain lesions by MRI can be quite heterogeneous in different patients, even resembling brain or spinal cord tumors in some. In addition, a normal MRI scan does not rule out a diagnosis of MS, as a small number of patients with confirmed MS do not show any lesions in the brain on MRI. These individuals often have spinal cord lesions or lesions which cannot be detected by MRI. As a result, it is critical that a thorough clinical exam also include a patient history and functional testing. This should cover mental, emotional, and language functions, movement and coordination, vision, balance, and the functions of the five senses. Sex, birthplace, family history, and age of the person when symptoms first began are also important considerations. Other tests, including evoked potentials (electrical diagnostic studies), cerebrospinal fluid, and blood (to rule out other causes), may be required in certain cases. Currently, there are no genetic markers that permit the diagnosis of MS. To date, the best association in large population studies with the risk of having MS is found in genomic screens within the HLA (chromosome 6) region, but it is clear that this association, albeit germane to a relative genetic predisposition, is insufficient even to suggest a diagnosis.

II. MS-Related Genes

In the following pages, applicants set forth those gene targets that may be used to diagnose/prognose MS based upon the relevant comparison. Also included are the particular probes utilized.

In the following tables, a plus (+) sign next to a probe set or gene name indicates higher expression observed in patients with MS (Tables 1-12, and 16), highlighting its diagnostic or prognostic value for MS, or it signifies that the gene or probe set is downregulated in MS patients that are receiving Avonex (the latter only applies to Tables 13-15). For example, thrombospondin 4 (+) (Table 13) indicates that this gene is higher in MS patients that in MS patients receiving Avonex. It suggests that Avonex may downregulate this inflammation-related gene and therefore thrombospondin 4 could be a target molecule to devise therapies for MS. Similarly, E2F-3 (+) (Table 15) means that this pro-proliferative transcription factor for immune cells is downregulated by Avonex, suggesting that manipulating the E2F pathway may have beneficial effects in MS.

The numbers under “Probe sets” represent the GenBank accession numbers or, in some instances, an identifier provided by Affymetrix.

TABLE 1
Highly discriminatory genes for MS (on no treatment) vs. Healthy Donors

| Probe sets | Gene Descriptions | Up (+) or Down (−) |
|---|---|---|
| D30037 | phosphatidylinositol transfer protein (PITPN) | + |
| D29675 | Inducible nitric oxide synthase (iNOS) | + |
| U78095 | Placental bikunin (AMBP) | + |
| U18271 | thymopoietin (TMPO) | + |
| D64158 | cell differentiation-associated ATP binding protein | + |
| Z25884 | CIC-1 muscle chloride channel protein (CLCN1) | + |
| U14187 | receptor tyrosine kinase ligand LERK-3/Ephrin-A3 | + |
| Z16411 | phospholipase c; Also: U26425, Z37544 (PLCB3) | − |
| U79528 | SR31747 binding prot 1 (SRBP1); Also: U75283 | + |
| X72879 | 14A2AK DNA sequence | + |
| L34357 | GATA4 | + |
| M76424 | carbonic anhydrase VII (CA VII) | + |
| M25269 | tyrosine kinase (ELK1) | + |
| HG2415- | Transcription Factor E2F2 | + |
| HT2511 | | |
| Z49254 | L23-related MRPL23 | − |
| HG3175- | Carcinoembryonic Antigen | + |
| HT3352 | | |
| Z78289 | (clone 1D2)/Z78289 | + |
| Z15114 | protein kinase C gamma (PRKCG) | + |
| AC002486 | BAC clone RG367O17/7p15-p21/AC002486 | + |
| HG3991- | Cpg-Enriched Dna, Clone E18 | + |
| HT4261 | | |
| D26069 | Centaurin beta 2 (CENTB2)/KIAA0041 | + |
| U25975 | serine kinase (hPAK65) | + |
| D87450 | parallel sister chromatids drosophila protein-like/KIAA0261 | + |
| S68874 | EP3 prostanoid receptor EP3-I | + |
| D45132 | kidney zinc-finger DNA-binding protein PRDM2 | + |
| Y09443 | alkyl-dihydroxyacetonephosphate synthase AGPS | + |
| M13994 | BCL2-alpha; Also: M14745 | + |
| X60483 | H4/D histone | + |
| M98833 | ERGB TRANSCRIPTION FACTORS (FLI-1 homolog) | + |
| U79303 | clone 23882 | + |
| Z47038 | putative microtubule-associated; protein 1A (MAP1A) | + |
| HG3638- | Amyloid Beta (A4) Precursor prot; Also: Y00264 | − |
| HT3849 | | |
| U03398 | receptor 4-1BB ligand (TNFSF9) | + |
| U66059 | TCRBV1S1A1N1 from germline T-cell receptor beta chain | + |
| HG2825- | Ret Transforming | + |
| HT2949 | | |
| L36922 | Met-ase 1/Granzyme M (GZMM) | + |
| U39318 | E2 ubiquitin conjugating enzyme UbcH5C (UBCH5C) | + |
| U49837 | LIM prot MLP (CSRP3) | + |
| U31903 | CREB-RP (CREBL1); Also: U89337_1, X98054 | + |
| X80878 | R kappa B (NFRKB) | + |
| D30036 | phosphatidylinositol transfer protein (PITPN) | + |
| S76992 | VAV2 | + |
| D83779 | Hypothetical protein/KIAA0195 | + |
| D26579 | transmembrane prot ADAM8 | + |
| X90857 | (−14) containing globin regulatory element (CGTHBA) | − |
| X13444 | CD8 beta-chain glycoprot (CD8 beta1) | + |
| M30818 | interferon-induced cellular resistance mediator MxB (MX2) | + |
| M19650 | 2,3-cyclic nucleotide 3-phosphodiesterase (CNP) | + |
| HG4108- | Olfactory Receptor Or17-24 | + |
| HT4378 | | |
| X89067 | trpc2 transcript | + |
| X83492 | Fas/APO1 (TNFRSF6) | + |
| L19593 | IL-8 receptor beta (IL8RB) | + |
| D49490 | disulfide isomerase-related protein (PDIR) | + |
| X89267 | DNA uroporphyrinogen decarboxylase (UROD) | + |
| D16217 | Calpastatin (CAST) | + |
| L05512 | histatin 1 (HTN1) | + |
| D38024 | facioscapulohumeral muscular dystrophy (FSHD) | + |
| U79262 | Deoxyhypusine | + |
| D38491 | Hypothetical protein/KIAA0117 | + |
| M28879 | granzyme B/CTLA-1 (GZMB) | + |
| Y10936 | hypothetical protein downstream of DMPK and DMAHP | + |
| Y09022 | Not56-like protein (NOT56L) | + |
| L35253 | p38 mitogen activated prot (MAP) kinase; Also: L35264 | + |
| HG644- | Histone H1.1 (HIST1H1A) | − |
| HT644 | | |
| S50017 | 2,3-cyclic nucleotide 3-phosphodiesterase (CNP) | + |
| X75755 | PR264; Also: HG3088-HT3261 | + |
| M29971 | 6-O-methylguanine-DNA methyltransferase (MGMT) | − |
| X07619 | cytochrome P450 db1 variant b; Also: X16866 | + |
| U53347 | neutral amino acid transporter B (SLC1A5) | + |
| M21259 | Alu repeats in the region to the Snrp E | − |
| S82597 | UDP-GaINAc: N-acetylgalactosaminyltransferase GALNT1 | + |
| U78521 | immunophilin homolog ARA9 | + |
| U47635 | D13S824E locus | − |
| D28364 | annexin II (ANXA2) | + |
| U59752 | Sec7p-like protein (PSCD2L) | + |
| Z19585 | thrombospondin-4 (THBS4) | + |
| Y00282 | ribophorin II (RPN2) | − |
| U43431 | DNA topoisomerase III alpha (TOP3A) | − |
| U63541 | expressed in HC/HCC livers and MoIT-4 proliferating cells | + |
| S81957 | bone morphogenic protein 5 (BMP5) | + |
| D38550 | E2F3 transcription factor/KIAA0075 | + |
| D38449 | G protein-coupled receptor (GPR) | + |
| D38524 | 5-nucleotidase (NT5C2) | + |
| HG4114- | Olfactory Receptor Or17-209 | + |
| HT4384 | | |
| D86979 | hypothetical protein/KIAA0226 | + |
| U57341 | neurofilament L (NFL) | + |
| D49487 | obese/Leptin (LEP); Also: U43653 | + |
| D86043 | SHPS-1/PTPNS1 tyrosine phosphatase; Also: U06701 | − |
| L76200 | guanylate kinase (GUK1) | − |
| U20647 | zinc finger protein (ZNF151) | + |

TABLE 2
Intermediate discriminatory genes for MS
(on no treatment) vs. Healthy Donors

| | | |
|---|---|---|
| L34075 | FKBP-rapamycin associated prot (FRAP) | + |
| U07807 | metallothionein IV (MTIV) | + |
| X65977 | corticostatin HP-4 precursor (defensin/DEFA4) | + |
| X05309 | C3b/C4b receptor (CR1) F allotype. | + |
| D87076 | Br140/KIAA0239 | + |
| D42046 | DNA replication helicase-like homolog/KIAA0083 | + |
| U15173 | Nip2 (NIP2) | + |
| U07550 | Chaperonin 10 (HSPE1) | − |
| HG2479- | Helix-Loop-Helix prot Sef2-1D; Also: M74719 | + |
| HT2575 | | |
| HG4094- | Transcription Factor LSF-Id; Also: U03494 | + |
| HT4364 | | |
| X16316 | VAV1 | + |
| U41068 | retinoid X receptor beta (RXRbeta) 3 | + |
| U43030 | cardiotrophin-1 (CTF1) | + |
| D82344 | NBPhox (PHOX2B) | + |
| U34301 | Nonmuscle myosin heavy chain IIB | + |
| HG3730- | Tyrosine Kinase Syk; Also: L28824 | + |
| HT4000 | | |
| M20778 | alpha-3 (VI) collagen; Also: X52022 | + |
| AB001325 | Aquaporin 3 (AQP3) | + |
| D28114 | myelin-associated oligodendrocytic basic protein | + |
| | (MOBP) | |
| U52154 | G-coupled inwardly rectifying potassium channel KIR34 | + |
| X79865 | Mrp17/Mitochondrial ribosomal L12 (MRPL12) | − |
| U76388 | steroidogenic factor 1 (NR5A1) | + |
| D31883 | actin-binding LIM protein 1 (ABLIM1)/KIAA0059 | + |
| Z14244 | coxVIIb cytochrome c oxidase subunit VIIb (COX7B) | − |
| S70348 | integrin beta 3 (ITGB3) | + |
| U56998 | putative serine/threonine protein kinase PRK | + |
| U33921 | HSU33921 cDNA | + |
| U89336 | Notch 4 | + |
| U79287 | clone 23867/prostate tumor overexpressed 1 (PTOV1) | − |
| D88613 | HGCMa/glial cells missing homolog 1 (GCM1) | + |
| L41607 | beta-16-N-acetylglucosaminyltransferase (IGNT) | + |
| S82362 | hRAR-beta 2 = retinoic-acid-receptor beta | + |
| U02566 | receptor tyrosine kinase TIF; Also: U18934 | − |
| AF001359 | mismatch repair protein (MLH1)/AF001359 | − |
| L27943 | cytidine deaminase (CDA) | + |
| U48405 | G prot coupled receptor OGR1 | + |
| M60750 | histone H2B.1 | + |
| U15197 | histo-blood group ABO protein | + |
| X13967 | leukemia inhibitory factor (LIF/HILDA) | + |
| U71364 | serine protease inhibitor (P19) | + |
| L42611 | keratin 6 isoform K6e (KRT6E) | + |
| U91930 | AP-3 complex delta subunit (AP3D1) | − |
| L77561 | DiGeorge syndrome critical region 11 DGS-D (DGCR11) | + |
| X90761 | Keratin, hair, acidic, 2 hHa2 (KRTHA2) | + |
| X02176 | complement component C9; Also: K02766 | − |
| U22028 | cytochrome P450 (CYP2A13) | + |
| S77583 | HERVK10/HUMMTV reverse transcriptase homolog | + |
| L36529 | (clone N5-4) protein p84 (THOC1) | − |
| M13577 | myelin basic protein (MBP) | + |
| U75272 | gastricsin/progastricsin (PGC); Also: J04443 | + |
| D89859 | zinc finger 5 protein (ZNF5) | + |
| Z75330 | nuclear protein stromal antigen SA-1 (STAG1) | + |
| M35416 | GTP-binding protein (RALB) | + |
| S76617 | protein tyrosine kinase (BLK) | + |
| U09303 | T cell leukemia LERK-2 (EPLG2)/Ephrin-B1 | + |
| D80004 | Hypothetical protein/KIAA0182 | + |
| Z18956 | taurine transporter (SLC6A6) | + |
| AB000464 | RES4-24A | + |
| X13461 | calmodulin-like protein (CLP); Also: M58026 | + |
| D79993 | Hypothetical/KIAA0171/Enthoprotin (ENTH) | + |
| Z46632 | HSPDE4C1 3,5-cyclic AMP phosphodiesterase (PDE4C) | + |
| AB002315 | Hypothetical protein/KIAA0317 | + |
| X57206 | 1D-myo-inositol-trisphosphate 3-kinase B (ITPKB) | + |
| L10717 | T cell-specific tyrosine kinase | + |
| D14889 | small GTP-binding protein S10 (RAB33A) | + |
| U46461 | Dishevelled homolog (DVL) | + |
| L42563 | (clone ISW34) non-gastric HK-ATPase (ATP1AL1) | + |
| M93405 | methylmalonate semialdehyde dehydrogenase | + |
| | (ALDH6A1) | |
| U68233 | farnesol receptor (HRR-1) | + |
| M34181 | testis-specific cAMP-dependent prot kinase (PRKACB) | + |
| L19183 | MAC30 hypothetical protein | − |
| HG2602- | Succinate Dehydrogenase Flavoprotein (HSSUCCDH) | + |
| HT2698 | | |
| D28416 | esterase D (ESD) | + |
| L20860 | glycoprotein lb beta (GP1BB) | + |
| L13977 | prolylcarboxypeptidase (PRCP) | − |
| U50553 | helicase like protein 2 (DDX3) | + |
| L14754 | DNA-binding protein (SMBP2) | + |
| Z47727 | RNA polymerase II subunit | + |
| HG3995- | Cpg-Enriched Dna Clone S19 | + |
| HT4265 | | |
| U61500 | GT334 prot (GT334) | + |
| L19058 | glutamate receptor (GLUR5) | + |
| HG4318- | Lim-Domain Transcription Factor Lim-1 (LHX1); | + |
| HT4588 | U14755 | |
| M14159 | T-cell receptor beta-chain J2.1 | + |
| K02574 | purine nucleoside phosphorylase (PNP) | + |
| U79261 | clone 23959 (MAPK8IP2); Also: U62317 | + |
| D83174 | collagen binding prot 2 (SERPINH1); Also: X61598 | − |
| M60626 | N-formylpeptide receptor 1 (FPR1) | + |
| Y09392 | WSL-LR, WSL-S1 and WSL-S2 prots; Also: U74611 | + |
| U24704 | antisecretory factor-1 (PSMD4) | − |
| U89505 | Hlark | − |
| D14661 | Wims' tumor-1 associating WTAP protein/KIAA0105 | + |
| U05040 | FUSE binding protein (FUBP1) | + |
| S74728 | antiquitin (ALDH7A1) | − |
| U40714 | tyrosyl-tRNA synthetase (YARS) | + |
| M28219 | low density lipoprotein receptor | + |
| Z25535 | nuclear pore complex protein hnup153 | + |
| U89896 | casein kinase I gamma 2 (CSNK1G2) | + |
| X71125 | glutamine cyclotransferase (QPCT) | + |
| S72487 | Platelet-derived endothelial growth factor 1 (ECGF1) | + |

TABLE 3
Least discriminatory genes for MS
(on no treatment) vs. Healthy Donors

| | | |
|---|---|---|
| U78575 | phosphatidylinositol 4-phosphate 5-kinase alpha | + |
| | PIP5K1A | |
| K01884 | Blym-1 transforming | + |
| X75304 | Giantin | − |
| M13699 | ceruloplasmin (CP) | + |
| Z49269 | chemokine HCC-1 (CCL14) | + |
| D28588 | Sp2 transcription factor/KIAA0048 | + |
| HG4115- | Olfactory Receptor Or17-210 | + |
| HT4385 | | |
| HG1751- | CSH5 | + |
| HT1768 | | |
| L42324 | (clone GPCR W) G protein-linked receptor/L42324 | + |
| J03764 | Plasminogen activator inhibitor 1 | + |
| Z26256 | L-type calcium channel | + |
| M13955 | Mesothelial keratin K7 (KRT7) | + |
| Y14140 | G protein-encoding beta 3 subunit 1 (GNB3) | + |
| X54871 | ras-related prot RAB5B | + |
| AD000092 | RAD23A homolog | + |
| D13988 | rab GDI | + |
| U58130 | Bumetanide-sensitive Na-K-2Cl cotransporter | + |
| | (NKCC2) | |
| U50743 | NaK-ATPase gamma subunit | + |
| Y13618 | DFFRY prot | + |
| U67932 | cAMP phosphodiesterase (PDE7A2); Also: L12052 | + |
| D86967 | alpha mannosidase-like protein/KIAA0212 | + |
| X05345 | histidyl-tRNA synthetase (HRS) | − |
| M91036 | G-gamma globin (HBG2) | + |
| U94319 | Autoantigen DFS70 | + |
| U02680 | protein tyrosine kinase 9 (PTK9) | − |
| X07203 | CD20 receptor (S7) | + |
| D78361 | ornithine decarboxylase antizyme 1 (OAZ1) | + |
| X98248 | Sortilin (SORT1) | + |
| M93221 | macrophage mannose receptor (MRC1) | − |
| AC002045 | Nuclear pore complex-interacting protein (NPIP) | + |
| D83784 | C2 H2-type zinc finger protein/KIAA0198 | + |
| AF005775 | caspase-like apoptosis regulatory prot 2 (CLARP) | + |
| Y10514 | CD152 prot (CTLA4); Also: Y10508 | − |
| X80907 | phosphatidyl-inositol-3-kinase p85 (PIK3R2) | + |
| U88666 | SFRS protein kinase 2 (SRPK2) | + |
| HG3104- | Serine Protease MET1 | + |
| HT3280 | | |
| U63717 | osteoclast stimulating factor (OSTF1) | + |
| D79990 | Ras association domain family 2/KIAA0168 | + |
| L32831 | G protein-coupled receptor (GPR3); Also: U18550 | + |
| U00238 | glutamine PRPP amidotransferase (GPAT) | − |
| L05425 | Autoantigen | − |
| U71601 | zinc finger protein zfp47 (ZNF47) | − |
| U51010 | nicotinamide N-methyltransferase 1 (NNMT); U08021 | − |
| U83410 | CUL-2 | + |
| U44799 | U1-snRNP binding prot homolog; Also: U44798 | + |
| L07261 | alpha adducin (ADD1) | + |
| X97303 | Ptg-12 protein | + |
| D31797 | CD40 ligand (TNFSF5) | + |
| HG3925- | SFTPA2D | + |
| HT4195 | | |
| L15309 | zinc finger protein (ZNF141) | + |
| X13255 | dopamine beta-hydroxylase (DBH) | + |
| Z69043 | translocon-associated prot delta subunit | − |
| | precursor (SSR4) | |
| J04162 | leukocyte IgG receptor (Fc-gamma-R) | + |
| U37431 | HOX A1 | + |
| L40397 | clone S31i125 | − |
| D10495 | protein kinase C delta-type (PRKCD) | + |
| U20938 | Lymphocyte dihydropyrimidine | + |
| | dehydrogenase (DPYD) | |
| U18919 | chromosome 17q12-21 clone pOV-2 | − |
| Y07827 | butyrophilin (BTN)/U90552 | + |
| U10868 | aldehyde dehydrogenase ALDH7 | + |
| U79252 | clone 23679 | + |
| M90299 | Glucokinase (GCK) | + |
| D56495 | Reg-related sequence derived peptide-2 (REGL) | + |
| X66839 | MaTu MN p54/58N carbonic anhydrase | + |
| | 9 protein (CA9) | |
| D25217 | Membrane protein MLC1/KIAA0027 | + |
| HG2715- | Tyrosine Kinase | + |
| HT2811 | | |
| L10338 | sodium channel beta-1 subunit | + |
| | (SCN1B)/U12194, L16242 | |
| D87969 | CMP-sialic acid transporter (SLC35A1) | + |
| L20815 | S protein/corneodesmosin (CDSN) | + |
| D87460 | Paralemmin/KIAA0270 | + |
| M60830 | Ecotropic viral integration site 2B (EVI2B) | + |
| X06745 | DNA polymerase alpha-subunit (POLA) | − |
| U02683 | Nuclear respiratory factor 1 (NRF1); Also: | + |
| | L22454, U18383 | |
| M80629 | cdc2-like protein kinase 5 (CDC2L5) | + |
| U38291 | Microtubule-associated prot 1a (MAP1A) | − |
| D16626 | Histidine ammonia lyase (HAL) | + |
| L39059 | TRANSCRIPTION FACTOR SL1 (TAF1C) | − |
| M31523 | transcription factor (E2A) | − |
| D50926 | Hypothetical protein/KIAA0136 | + |
| U82535 | fatty acid amide hydrolase (FAAH) | + |
| U02310 | k head domain protein (FKHR) | + |
| AJ000480 | C8FW phosphoprotein | + |
| HG627- | Rhesus (Rh) Blood Group Ce-Antigen 2, Rhvi/X63097 | + |
| HT5097 | | |
| S69790 | Brush-1 = tumor suppressor | + |
| U27325 | thromboxane A2 receptor (TBXA2R); Also: D38081 | + |
| X66867 | MAX | + |
| X82634 | hair keratin acidic 3B (KRTHA3B) | + |
| HG4018- | Opioid-Binding Cell Adhesion Molecule | + |
| HT4288 | | |
| M80244 | E16 | + |
| U10886 | Protein tyrosine phosphatase (PTPRJ) | + |
| U51903 | RasGAP-related protein (IQGAP2) | + |
| AF007111 | MDM2-like p53-binding protein (MDMX) | + |
| M32886 | sorcin CP-22 (SRI) | − |
| J04809 | Cytosolic adenylate kinase AK1 | + |
| U80811 | Lysophosphatidic acid receptor homolog (EDG2) | − |
| X04445 | Inhibin alpha (INHA); Also: M13981 | − |
| X06323 | MRL3 ribosomal prot L3 homolog | + |
| S78432 | un-named-transcript-1 from SAS = | − |
| | transmembrane 4 protein | |
| S71129 | acetylcholinesterase (ACHE) | + |
| X82240 | T cell leukemia/lymphoma 1 (TCL1A) | + |
| Y08999 | Sop2p-like protein | − |
| U51561 | cosmid N79E2 sequence | + |
| D37781 | protein-tyrosine phosphatase (PTPRJ); Also: U10886 | + |
| X97230 | NK receptor, clone library 4M1#6 | + |
| U48936 | amiloride-sensitive epithelial sodium | + |
| | channel gamma subU | |
| U81006 | p76 transmembrane 9 superfamily | + |
| | member 2 (TM9SF2) | |
| X98534 | Vasodilator-stimulated phosphoprotein | + |
| | (VASP); Z46389 | |
| L20859 | leukemia virus receptor 1 GLVR1 (SLC20A1) | + |
| X94563 | DBI/ACBP | + |
| U70323 | ataxin-2 (SCA2) | − |
| U39840 | Hepatocyte nuclear factor-3 alpha (HNF-3 alpha) | + |
| U78190 | GTP cyclohydrolase I feedback regulatory | + |
| | prot (GCHFR) | |
| X55330 | aspartylglucosaminidase (AGA) | − |
| U05237 | fetal Alz-50-reactive clone 1 (FAC1) | + |
| M31210 | Endothelial differentiation protein (EDG-1) | + |
| D63998 | golgi alpha-mannosidase II (MAN2A1) | + |
| U52077 | mariner1 transposase complete consensus/U80776 | − |
| U06454 | AMP-activated protein kinase (hAMPK) | + |
| U90916 | clone 23815 sequence, IFN-inducible | + |
| U03399 | T-complex protein 10A (TCP10A) | + |
| HG2668- | Bradykinin Receptor | + |
| HT2764 | | |
| L76568 | S26 from excision and cross link | − |
| | repair protein (ERCC4) | |
| HG64- | NF-Kappa B-Binding protein (KBP1) | − |
| HT64 | | |
| Z68193 | Opsin 1 (OPN1LW) | + |
| X58298 | interleukin-6-receptor (IL6R); Also: M20566 | + |
| Z12962 | homolog to yeast ribosomal protein L41 | + |
| Y08265 | DAN26 protein; Also: U94836 | + |
| U31248 | zinc finger protein (ZNF174) | − |
| S72503 | Inwardly rectifier potassium channel (KCNJ4); | + |
| | U07364 | |
| S76473 | tyrosine kinase receptor trkB (NTRK2); U12140 | + |
| K03218 | src sarcoma viral oncogene homolog (SRC) | + |
| Z48923 | Bone morphogenetic protein receptor 2 (BMPR2) | + |
| U21049 | Membrane-associated protein 17 (DD96) | + |
| HG3521- | Ras-Related protein 1b (RAP1B) | + |
| HT3715 | | |
| U24685 | anti-B cell autoantibody IgM heavy | + |
| | chain V-D-J region | |
| D45371 | GS3109 adipose most abundant gene transcript 1 | + |
| | (APM1) | |
| M17466 | blood coagulation factor XII (F12) | − |
| M80899 | novel protein desmoyokin (AHNAK) | + |
| X83492 | Fas/APO1 (TNFRSF6); Also: X63717, X83490 | + |
| U33920 | clone lambda 5 semaphorin 3F (SEMA3F) | + |
| V00594 | metallothionein 2A (MT2A); Also: J00271 | − |
| U20428 | SNC19 sequence | + |
| HG4272- | Hepatocyte Growth Factor Receptor | + |
| HT4542 | | |
| M30607 | zinc finger protein Y-linked (ZFY); Also: L10393 | − |
| M24485 | glutathione S-transferase pi (GSTP1); Also: U21689 | − |
| U34976 | gamma-sarcoglycan (SGCG) | − |
| D85376 | DNA thyrotropin-releasing hormone receptor (TRHR) | + |
| D50532 | macrophage lectin 2 (HML2) | + |
| AB000897 | cadherin FIB3 | + |
| M29994 | alpha-I spectrin (SPNA1); Also: M61877, M61826 | + |
| D42138 | Phosphatidylinositol glycan type B (PIG-B) | + |
| X66142 | rod cGMP phosphodiesterase 6b (PDE6B); Also: | + |
| | S41458 | |
| U10686 | MAGE-11 antigen (MAGEA11) | + |
| HG3994- | Cpg-Enriched DNA Clone S16 | + |
| HT4264 | | |
| X97324 | adipophilin (ADFP) | + |
| U18235 | ATP-binding cassette prot (ABC2) HFBCD04 clone | + |
| X76105 | Death-associated protein (DAP1) | − |
| M65290 | Interleukin 12p40 (IL12p40/IL12B) | + |
| D63160 | DNA lectin P35/Ficolin 2 (FCN2) | + |
| AB006190 | aquaporin 6 (AQP6) | + |
| L42572 | p87/89 mitochondrial inner membrane protein (IMMT) | − |
| U35113 | metastasis-associated MTA1 | − |
| U03634 | P47 LBC oncoprotein | + |
| X98253 | ZNF183/X98253 | + |
| D90276 | CGM7 nonspecific cross-reacting antigen (NCA) | + |
| Y08263 | AAD14 prot | + |
| HG4102- | N-Ethylmaleimide-Sensitive Factor (NSF) | + |
| HT4372 | | |
| HG4638- | Spliceosomal protein Sap 49 | + |
| HT5050 | | |
| U37408 | C-terminal binding protein 1 (CTBP1) | − |
| HG3748- | Basic Transcription Factor 44 Kda Subunit | + |
| HT4018 | | |
| U49835 | YKL-39 precursor; chitinase 3-like 2; | + |
| | U58514, U58515 | |
| U37055 | hepatocyte growth factor-like | − |
| | macrophage-stimulating 1 | |
| L40395 | clone S20iii15 | + |
| X90530 | ragB protein | + |
| M24461 | pulmonary surfactant-associated protein SP-B (SFTP3) | + |
| D13639 | G1/S-specific cyclin D2/KIAK0002 | + |
| M35999 | platelet glycoprot IIIa/Integrin beta 3 (ITGB3) | + |
| X13589 | aromatase (estrogen synthetase) (CYP19A1) | + |
| U52827 | Cri-du-chat region clone NIBB11 | + |
| X79353 | XAP-4 GDP-dissociation inhibitor (GDI1) | − |
| U00115 | zinc-finger protein (BCL6) | − |
| X78686 | Chemokine (C-X-C motif) ligand 5 (CXCL5) | + |
| HG3627- | Calcium Channel Voltage-Gated Beta 1 | + |
| HT3836 | Subunit L Type 2 | |
| Y00486 | adenine phosphoribosyltransferase (APRT) | + |
| D50918 | septin 2, 6 (SEPT6)/KIAA0128 | + |
| X06318 | protein kinase C (PKC) type beta I (PRKCB) | + |
| X52730 | phenylethanolamine n-methyltransferase (PNMT) | + |
| AB000896 | cadherin FIB2 | + |
| X81892 | G protein-couped receptor 64 (GPR64) | + |
| D25538 | adenylate cyclase 7 (ADCY7)/KIAA0037 | + |
| X68836 | S-adenosylmethionine synthetase (MAT2A) | + |
| X52638 | 6-phosphofructo-2-kinase/fructose-26-bisphosphatase | + |
| L16842 | ubiquinol cytochrome-c reductase core I | − |
| | prot (UQCRC1) | |
| D30758 | Centaurin beta 1 (CENTB1)/KIAA0050 | + |
| X53586 | integrin alpha 6 VLA6 (ITGA6) | + |
| X58199 | adducin 2 beta (ADD2); Also: S81083_1 | + |
| S50223 | HKR-T1 = Kruppel-like zinc finger prot | + |
| X52011 | MYF6 encoding a muscle determination factor | + |
| D42108 | phospholipase C-like 1 (PLCL1) | + |
| D88795 | Cadherin | + |
| HG3517- | Alpha-1-Antitrypsin | + |
| HT3711 | | |
| M16714 | MHC class I divergent lymphocyte antigen; clone RS5 | + |
| X76061 | p130 retinoblastoma-like 2 (RBL2) | + |
| U79301 | clone 23842 sequence | + |
| U80040 | aconitase nuclear encoded mitochondrial | − |
| | protein (ACO2) | |
| U82311 | unknown protein/U82311 | + |
| U79294 | clone 23748 phosphatidic acid phosphatase | + |
| | 2B (PPAP2B) | |
| X99664 | prot containing SH3 domain SH3GL3 | + |
| U00928 | clone CE29 4.1 (CAC)n/(GTG)n repeat-containing | + |
| S41458 | rod cGMP phosphodiesterase 6B (PDE6B) | + |
| U06863 | follistatin-related protein precursor (FSTL1) | + |
| HG3884- | Homeotic protein HPX42 | − |
| HT4154 | | |
| M12759 | Ig J chain (IGJ) | + |
| Y10517 | CD108 prot/Y10517 | + |
| U09002 | N-methyl-D-aspartate receptor subunit 2A (GRIN2A) | + |
| HG627- | Rhesus (Rh) Blood Group Ce-Antigenl, 3, | − |
| HT5098 | Rhviii/X63097 | |
| M81933 | Cell division cycle 25A (CDC25A) | + |
| X98263 | M-phase phosphoprot mpp6 | + |
| U90905 | clone 23574 sequence | + |
| X67683 | keratin 4 (KRT4) | + |
| HG315- | Beta-1-Glycoprot 11 Pregnancy-Specific (PSG11) | + |
| HT315 | | |
| M77144 | 3-b-hydroxysteroid dehydrogenase/5delta-4delta | + |
| | isomerase | |
| D85418 | phosphatidylinositol-glycan-class C (PIGC) | + |
| M95549 | sodium/glucose cotransporter-like protein (SLC5A2) | + |
| X16665 | HOX2H | + |
| AF002224 | E6-AP ubiquitin prot ligase 3A (UBE3A) | + |
| U12779 | MAP kinase activated protein kinase 2 (MAPKAPK2) | + |
| U07919 | aldehyde dehydrogenase 6 | + |
| S85655 | prohibitin (PHB) | − |
| U09087 | thymopoietin (TMPO); Also: U09088 | + |
| U72512 | B-cell receptor associated protein (hBAP) | + |
| U52112 | Renin binding protein (RENBP) | + |
| HG4747- | Nadh-Ubiquinone Oxidoreductase 51 Kda Subunit | + |
| HT5195 | | |
| AB000450 | Vaccinia related kinase 2 (VRK2) | + |
| X12794 | v-erbA related ear-2/NR2F6 | + |
| M19645 | 78 kdalton glucose-regulated protein (GRP78) | − |
| M10277 | actin (ACTB) | + |
| D87074 | rab3 interacting protein/KIAA0237 | + |
| U58033 | myotubularin related prot 2 (MTMR2)/U58033 | + |
| HG2167- | prot Kinase Ht31 cAMP-Dependent | + |
| HT2237 | | |
| U41371 | spliceosome associated prot (SAP 145) | − |
| U49395 | ionotropic ATP receptor P2X5a | + |
| X64624 | RDC-1 POU domain containing prot; Also: L20433 | + |
| U18548 | GPR12 G protein coupled-receptor | + |
| U55853 | 130 kD Golgi-localized phosphoprot (GPP130) | + |
| Z70759 | mitochondrial 16S rRNA ()/Z70759 | + |
| L04953 | Amyloid precursor protein binding APBA1 | + |
| U68142 | RaIGDS-like 2 (RGL2) | + |
| M16279 | MIC2 | − |
| U70732 | glutamate pyruvate transaminase (GPT) | + |
| M31520 | Ribosomal S24 | − |
| D28423 | pre-splicing factor SRp20 | + |
| X65857 | HGMP07E olfactory receptor | + |
| U30313 | diadenosine tetraphosphatase (NUDT2) | − |
| U78678 | thioredoxin 2 (TXN2) | + |
| J05582 | Mucin 1 (MUC1); Also: J05581 | − |
| U85767 | myeloid progenitor inhibitory factor-1 (MPIF1) | + |
| M94172 | N-type calcium channel alpha-1 subunit (CACNA1B) | + |
| AB002559 | hunc18b2 | + |
| X62078 | GM2 activator protein (GM2A) | − |
| M62831 | TRANSCRIPTION FACTORS ETR101 | + |
| Z18954 | S100D calcium binding prot | + |
| X54816 | alpha-1-microglobulin-bikunin (AMBP) | + |
| M35531 | fucosyltransferase (FUT1) | + |
| M90391 | Interleukin 16 (IL16) | + |
| M87284 | 69 kDa 2′ oligoadenylate synthetase | − |
| | (P69 2-5A synthetase) | |
| U91932 | AP-3 complex sigma 3A subunit (AP3S1) | − |
| D87673 | heat shock transcription factor 4 (HSF4) | + |
| D86976 | minor histocompatibility antigen HA-1/KIAA0223 | + |
| M82882 | cis-acting ELF1 | + |
| D83657 | calcium-binding prot in amniotic fluid 1 | + |
| | CAAF1 (S100A12) | |
| U41740 | trans-Golgi p230 (GOLGA4) | − |
| U12535 | epidermal growth factor receptor kinase | − |
| | substrate (EPS8) | |
| HG3635- | Zinc Finger prot, Kruppel-Like | + |
| HT3845 | | |
| D50915 | hypothetical protein/KIAA0125 | + |
| L04490 | NADH-ubiquinone oxidoreductase subunit (NDUFA9) | − |
| U59748 | desert hedgehog (DHH) | + |
| U82010 | heme A: farnesyltransferase (COX10) | − |
| D50402 | Natural resistance associated macrophate | + |
| | prot1 (NRAMP1) | |
| X16983 | integrin alpha-4 subunit VLA4 (ITGA4) | + |
| U68536 | zinc finger protein 45 (ZNF45); Also KOX17 | + |
| X80909 | alpha NAC nascent polypeptide-associated complex | − |
| M24248 | MLC-1V-Sb | + |
| X96401 | MAX-binding ROX protein | + |
| D31888 | CoREST protein (RCOR)/KIAA0071 | + |
| X95404 | non-muscle type cofilin | − |
| U09550 | oviductal glycoprotein (OVGP1) | + |
| S79219 | metastasis-associated; Also: X14608 | − |
| X65488 | Heterogeneous nuclear ribonucleoprotein U (HNRPU) | + |
| D87685 | TFIIS-like PHD finger protein 3/KIAA0244 | + |
| L02840 | potassium channel Kv21 (KCNB1) | + |
| Z19554 | vimentin (VIM); Also: M18895_2 | − |
| L36051 | Thrombopoietin (THPO) | + |
| M81758 | voltage-dependent sodium channel SKM1 (SCN4A) | + |
| HG3991- | Cpg-Enriched Dna, Clone E18 | + |
| HT4261 | | |
| D31846 | DRPLA | + |
| U19906 | arginine vasopressin receptor 1 (AVPR1) | + |
| AB002366 | hypothetical protein/KIAA0368 | − |
| M68520 | cyclin-dependent kinase 2 (CDK2) | + |
| U69546 | RNA binding prot ETR3 | + |
| U58970 | outer mitochondrial membrane translocase (TOMM34) | − |
| L31584 | G prot-coupled receptor (EBI1) | + |
| X60655 | EVX1 homeobox | + |
| U71300 | snRNA activating prot complex 50 kD | + |
| | subunit (SNAP50) | |
| S75578 | 4-aminobutyrate aminotransferase (ABAT) | − |
| X82693 | Lymphocyte antigen 6 complex, locus D (E48) | + |
| HG1155- | Colony-Stimulating Factor 1 Macrophage (CSF1) | + |
| HT4822 | | |
| D87432 | solute carrier family 7/KIAA0245 | + |
| D50640 | phosphodiesterase 3B (PDE3B) | + |
| D13370 | APEX nuclease (APEX1) | + |
| Z22548 | thiol-specific antioxidant peroxiredoxin 2 (PRDX2) | − |
| M33308 | Vinculin (VCL) | − |
| M80335 | protein kinase A catalytic subunit (PRKACA) | + |
| U14391 | myosin-IC | − |
| Y09943 | NGF-inducible PC3 anti-proliferative protein (BTG2) | + |
| M81780 | Sphingomyelin phosphodiesterase 1 (SMPD1) | + |
| U20582 | actin-like peptide (LOC81569) | + |
| D10656 | Sarcoma virus homolog (CRK) | + |
| Y08319 | kinesin-2 (KIF2) | + |
| Z48633 | retrotransposon | − |
| X15187 | homolog of murine tumor rejection | − |
| | antigen gp96 (TRA1) | |
| HG884- | E6-Associated protein, Papillomavirus | − |
| | (UBE3A); U84404 | |
| HT884 | | |
| X77753 | TROP-2 (TACSTD2) | + |
| D12620 | cytochrome P-450 LTBV; Also: U02388 | + |
| Y09615 | mitochondrial transcription termination | + |
| | factor (MTERF) | |
| J02883 | Colipase (CLPS) | + |
| M81637 | Grancalcin (GCA) | + |
| M61733 | erythroid membrane protein 41 (EPB41) | + |
| U23430 | Cholecystokinin type A receptor (CCKAR); | + |
| | Also: L19315 | |
| X95325 | DNA binding prot A variant; Also: M24069 | − |
| D87434 | Hypothetical protein/KIAA0247 | + |
| HG274- | Gamma-Glutamyltransferase 1 (GGT1); J04131 | − |
| HT274 | | |
| M98776 | keratin 1 | + |
| L20941 | ferritin heavy chain (FTH1) | − |
| D87292 | Rhodanese | + |
| K02766 | complement component C9 (C9) | − |
| X12447 | aldolase A (ALDOA) (EC 41213) | − |
| M59941 | GM-CSF receptor beta chain (CSF2RB) | + |
| D11327 | protein tyrosine phosphatase (PTPN7); Also: M64322 | + |
| X90846 | mixed lineage kinase 2 (MAP3K10) | + |
| U33822 | tax1-binding prot TXBP181, MAD1-like 1 (MAD1L1) | + |
| D49410 | interleukin 3 receptor alpha subunit (IL3RA) | + |
| L33842 | type II inosine monophosphate | − |
| | dehydrogenase (IMPDH2) | |
| U57352 | sodium channel 1 (hBNaC1) | + |
| J04444 | cytochrome c-1 (CYC1) | − |
| L13329 | iduronate-2-sulfatase (IDS) | + |
| D50919 | TRIM14 protein/KIAA0129 | + |
| L38935 | GT212 | + |
| L17327 | pre-T/NK cell associated prot (3B3) | + |
| X04143 | bone gla prot (BGP) | − |
| J05243 | nonerythroid alpha-spectrin (SPTAN1) | − |
| Z11502 | annexin A13 (ANXA13) | + |
| D13637 | toll-like receptor 1 (TLR1)/KIAA0012 | + |
| U32680 | CLN3 | + |
| D29954 | hypothetical protein/KIAA0056 | + |
| M61855 | cytochrome P4502C9 (CYP2C9), clone 25 | + |
| U82279 | Ig-like transcript 2 (LILRB1) | + |
| Z83336 | hH2B/d | + |
| U36759 | pre-T cell receptor alpha-type chain | + |
| | precursor (PTCRA) | |
| L13848 | RNA helicase A (DDX9) | + |
| U70862 | nuclear factor I/B (NFIB) | + |
| HG1102- | Ras-Related C3 Botulinum Toxin Substrate (RAC1) | − |
| HT1102 | | |
| D85181 | fungal sterol-C5-desaturase homolog (SC5DL) | + |
| M31642 | hypoxanthine phosphoribosyltransferase (HPRT) | − |
| D87470 | hypothetical protein/KIAA0280 | − |
| X74330 | DNA primase (PRIM1) | + |
| D28915 | hepatitis C-associated protein p44 (IFI44) | + |
| U82979 | Ig-like transcript-3 (LILRB4) | + |
| HG2480- | Fmlp-Related Receptor I | + |
| HT2576 | | |
| HG4263- | NKR-P1A protein | + |
| HT4533 | | |
| S77576 | ERV9 reverse transcriptase homolog | + |
| M25629 | kallikrein (KLK1) | − |
| M89957 | B cell receptor complex cell surface | + |
| | glycoprotein (IGB) | |
| X15875 | cAMP response element binding prot | − |
| | CREBP1 (ATF2) | |
| D50550 | Lethal giant larvae homolog 1 (LLGL1) | + |
| U80017 | Survival motor neuron protein (SMN) | − |
| L40387 | thyroid receptor interactor TRIP14 (OASL) | + |
| U34343 | 13 kD differentiation-associated protein (DAP13) | − |
| U04270 | putative potassium channel subunit (KCNH2) | − |
| X97160 | TFE3 transcription factor from TFE3 | + |
| D83018 | NEL-related protein 2 (NELL2) | + |
| AF008937 | syntaxin-16C | + |
| U38276 | semaphorin III family homolog (SEMA3F) | − |
| X83368 | phosphatidylinositol 3 kinase gamma (PIK3CG) | + |
| X59739 | Zinc finger protein, X-linked (ZFX) | + |
| J02611 | apolipoprotein D | + |
| X13293 | v-myb myeloblastosis viral homolog-like 2 (MYBL2) | − |

TABLE 4
Highly discriminatory genes for MS (regardless of
treatment with Avonex or not) vs. Healthy Donors

| Probe sets | Gene Descriptions | Up (+) or down (−) |
|---|---|---|
| Z16411 | phospholipase c; Also: U26425, Z37544 (PLCB3) | − |
| U78095 | Placental bikunin (AMBP) | + |
| L34075 | FKBP-rapamycin associated prot (FRAP) | + |
| X90857 | (−14) containing globin regulatory element (CGTHBA) | − |
| HG644- | Histone H1.1 (HIST1H1A) | − |
| HT644 | | |
| D86043 | SHPS-1/PTPNS1 tyrosine phosphatase; Also: U06701 | − |
| Z49254 | L23-related MRPL23 | − |
| M21259 | Alu repeats in the region to the small nuclear ribonucleoprot E | − |
| X13444 | CD8 beta-chain glycoprot (CD8 beta1) | + |
| Y00282 | ribophorin II (RPN2) | − |
| S74728 | antiquitin (ALDH7A1) | − |
| U79303 | clone 23882 | + |
| U15197 | Histo-blood group ABO prot | + |
| M76424 | carbonic anhydrase VII (CA VII) | + |
| M29971 | 6-O-methylguanine-DNA methyltransferase (MGMT) | − |
| X16316 | vav oncogene | + |
| D83174 | collagen binding prot 2 (SERPINH1); Also: X61598 | − |
| U07550 | Chaperonin 10 (HSPE1) | − |
| U39318 | E2 ubiquitin conjugating enzyme UbcH5C (UBCH5C) | + |
| L42324 | (clone GPCR W) G prot-linked receptor/L42324 | + |
| L19593 | IL-8 receptor beta (IL8RB) | + |
| M98833 | ERGB TRANSCRIPTION FACTORS (FLI-1 homolog) | + |
| M93221 | macrophage mannose receptor (MRC1) | − |
| M14159 | T-cell receptor beta-chain J2.1 | + |
| X05345 | histidyl-tRNA synthetase (HRS) | − |
| U66059 | TCRBV1S1A1N1 from germline T-cell receptor beta chain | + |
| D79993 | Hypothetical/KIAA0171/Enthoprotin (ENTH) | + |
| U24704 | antisecretory factor-1 (PSMD4) | − |
| X72879 | 14A2AK DNA sequence | + |
| D29675 | Inducible nitric oxide synthase (iNOS) | + |
| U94319 | autoantigen DFS70 | + |
| HG3175- | Carcinoembryonic Antigen | + |
| HT3352 | | |
| L20859 | leukemia virus receptor 1 GLVR1 (SLC20A1) | + |
| K01884 | Blym-1 transforming | + |
| S76992 | VAV2 = VAV onco homolog | + |
| HG2415- | Transcription Factor E2f-2 | + |
| HT2511 | | |
| L76200 | guanylate kinase (GUK1) | − |
| M25269 | tyrosine kinase (ELK1) onco | + |
| X05309 | C3b/C4b receptor (CR1) F allotype. | + |
| AC002486 | BAC clone RG367O17/7p15-p21/AC002486 | + |
| U79528 | SR31747 binding prot 1 (SRBP1); Also: U75283 | + |
| U89896 | casein kinase I gamma 2 (CSNK1G2) | + |
| X66867 | MAX | + |
| Y09392 | WSL-LR, WSL-S1 and WSL-S2 prots; Also: U74611 | + |
| Z15114 | protein kinase C gamma (PRKCG) | + |
| L36922 | Met-ase 1/Granzyme M (GZMM) | + |
| U79287 | clone 23867/prostate tumor overexpressed 1 (PTOV1) | − |
| M31523 | transcription factor (E2A) | − |
| U37055 | hepatocyte growth factor-like/L11924, M74178, U28055 | − |
| U52077 | mariner1 transposase complete consensus sequence/U80776 | − |
| D86979 | hypothetical protein/KIAA0226 | + |
| D26579 | transmembrane prot ADAM8 | + |
| Y09443 | alkyl-dihydroxyacetonephosphate synthase AGPS | + |
| U79262 | deoxyhypusine | + |
| U51903 | RasGAP-related prot (IQGAP2) | + |
| M60830 | Ecotropic viral integration site 2B (EVI2B) | + |
| U53347 | neutral amino acid transporter B (SLC1A5) | + |
| D79990 | Ras association domain family 2/KIAA0168 | + |
| Z47038 | microtubule-associated; prot 1A (MAP1A)/U38291_rna1 | + |
| U30313 | diadenosine tetraphosphatase (NUDT2) | − |
| D26069 | Centaurin beta 2 (CENTB2)/KIAA0041 | + |
| Z69043 | translocon-associated prot delta subunit precursor (SSR4) | − |
| U02683 | Nuclear respiratory factor 1 (NRF1); Also: L22454, U18383 | + |
| U46461 | dishevelled homolog (DVL) | + |
| U47635 | D13S824E locus | − |
| HG3991- | Cpg-Enriched Dna, Clone E18 | + |
| HT4261 | | |
| L27943 | cytidine deaminase (CDA) | + |
| U34976 | gamma-sarcoglycan (SGCG) | − |
| U31248 | zinc finger prot (ZNF174) | − |
| U18271 | thymopoietin (TMPO); Also: U09087, U09088 | + |
| HG627- | Rhesus (Rh) Blood Group Ce-Antigenl, 3, Rhviii; Also: X63097 | − |
| HT5098 | | |
| M19650 | 2,3-cyclic nucleotide 3-phosphodiesterase (CNP) | + |
| HG3730- | Tyrosine Kinase Syk; Also: L28824 | + |
| HT4000 | | |
| D13988 | rab GDI | + |

TABLE 5
Intermediate discriminatory genes for MS
(regardless of treatment with Avonex
or not) vs. Healthy Donors

| | | |
|---|---|---|
| D10495 | protein kinase C delta-type (PRKCD) | + |
| D64158 | cell differentiation-associated ATP binding prot | + |
| U78521 | immunophilin homolog ARA9 | + |
| D30037 | phosphatidylinositol transfer protein (PITPN) | + |
| U33921 | HSU33921 cDNA | + |
| HG2825- | Ret Transforming | + |
| HT2949 | | |
| L34357 | GATA-4 | + |
| L16842 | ubiquinol cytochrome-c reductase core I | − |
| | prot (UQCRC1) | |
| D16626 | Histidine ammonia lyase (HAL) | + |
| U48405 | G prot coupled receptor OGR1 | + |
| Y10514 | CD152 prot (CTLA4); Also: Y10508 | − |
| HG3521- | Ras-Related prot 1b | + |
| HT3715 | | |
| D86967 | alpha mannosidase-like protein/KIAA0212 | + |
| HG4318- | Lim-Domain Transcription Factor Lim-1 | + |
| HT4588 | (LHX1); U14755 | |
| X75304 | giantin | − |
| M19645 | 78 kdalton glucose-regulated prot (GRP78) | − |
| AB000464 | RES4-24A | + |
| U14187 | receptor tyrosine kinase ligand LERK-3/Ephrin-A3 | + |
| Z26256 | L-type calcium channel/Z26256 | + |
| U34301 | nonmuscle myosin heavy chain IIB/U34301 | + |
| HG4108- | Olfactory Receptor Or17-24 | + |
| HT4378 | | |
| X07619 | cytochrome P450 db1 variant b; Also: X16866 | + |
| Z14244 | coxVllb cytochrome c oxidase subunit VIIb (COX7B) | − |
| M90299 | glucokinase (GCK) | + |
| D78361 | ornithine decarboxylase antizyme 1 (OAZ1) | + |
| M62831 | TRANSCRIPTION FACTORS ETR101 | + |
| U50553 | helicase like protein 2 (DDX3) | + |
| D31883 | actin-binding LIM protein 1 (ABLIM1)/KIAA0059 | + |
| U44799 | U1-snRNP binding prot homolog; Also: U44798 | + |
| U80811 | Lysophosphatidic acid receptor homolog (EDG2) | − |
| D31797 | CD40 ligand (TNFSF5) | + |
| U89336 | Notch 4 | + |
| L04490 | NADH-ubiquinone oxidoreductase subunit (NDUFA9) | − |
| X52943 | ATF-a TRANSCRIPTION FACTORS | − |
| U91930 | AP-3 complex delta subunit (AP3D1) | − |
| U12535 | epidermal growth factor receptor kinase | − |
| | substrate (EPS8) | |
| X83492 | Fas/APO1 (TNFRSF6) | + |
| U89505 | Hlark | − |
| U59752 | Sec7p-like prot | + |
| X15187 | tra1 homolog of murine tumor rejection antigen gp96 | − |
| X72964 | caltractin | − |
| X58298 | interleukin-6-receptor (IL6R); Also: M20566 | + |
| U41371 | spliceosome associated prot (SAP 145) | − |
| U02310 | k head domain prot (FKHR) | + |
| X98248 | Sortilin (SORT1) | + |
| D50640 | phosphodiesterase 3B (PDE3B) | + |
| D55696 | cysteine protease | − |
| Z46632 | HSPDE4C1 3,5-cyclic AMP | + |
| | phosphodiesterase (PDE4C) | |
| Y09022 | Not56-like protein (NOT56L) | + |
| M32886 | sorcin CP-22 (SRI) | − |
| D16217 | Calpastatin (CAST) | + |
| D83018 | nel-related prot 2 | + |
| U76388 | steroidogenic factor 1 (NR5A1) | + |
| S85655 | prohibitin (PHB) | − |
| L33842 | (clone FFE-7) type II inosine | − |
| | monophosphate dehydrogenase | |
| Z49269 | chemokine HCC-1 (CCL14) | + |
| U63717 | osteoclast stimulating factor (OSTF1) | + |
| M82882 | cis-acting ELF1 | + |
| D45132 | kidney zinc-finger DNA-binding protein PRDM2 | + |
| Z18956 | taurine transporter (SLC6A6) | + |
| U03398 | receptor 4-1BB ligand (TNFSF9) | + |
| L05512 | histatin 1 (HTN1) | + |
| U82010 | heme A: farnesyltransferase (COX10) | − |
| U20428 | SNC19 sequence | + |
| U69546 | RNA binding prot ETR3 | + |
| S72503 | HRK1 = inward rectifier potassium | + |
| | channel; Also: U07364 | |
| M80335 | protein kinase A catalytic subunit (PRKACA) | + |
| U22028 | cytochrome P450 (CYP2A13) | + |
| M75099 | Rapamycin and FK506-binding protein FKBP13 | − |
| U68536 | zinc finger protein 45 (ZNF45); Also KOX17 | + |
| AB001325 | Aquaporin 3 (AQP3) | + |
| X65977 | corticostatin HP-4 precursor (defensin/DEFA4) | + |
| HG2167- | prot Kinase Ht31 Camp-Dependent | + |
| HT2237 | | |
| L21936 | succinate dehydrogenase flavoprot subunit (SDH) | − |
| L40397 | (clone S31i125) | − |
| AC002073 | WUGSC: DJ515N12/PAC clone | + |
| | DJ515N1/22q112-q22 | |
| D86976 | minor histocompatibility antigen HA-1/KIAA0223 | + |
| D28416 | esterase D (ESD) | + |
| D30036 | phosphatidylinositol transfer protein (PITPN) | + |
| X15875 | cAMP response element binding prot CREBP1 (ATF2) | − |
| U90913 | clone 23665 sequence | − |
| L33243 | polycystic kidney disease 1 prot (PKD1) | − |
| U80628 | thymidine kinase 2 isom B (TK2) alternatively spliced | − |
| U02566 | receptor tyrosine kinase TIF; Also: U18934 | − |
| D87460 | paralemmin/KIAA0270 | + |
| D11327 | protein tyrosine phosphatase (PTPN7); Also: M64322 | + |
| D87450 | parallel sister chromatids drosophila | + |
| | protein-like/KIAA0261 | |
| HG1862- | Calmodulin Type I | + |
| HT1897 | | |
| AF005775 | caspase-like apoptosis regulatory prot 2 (CLARP) | + |
| X75755 | PR264; Also: HG3088-HT3261 Same | + |
| | Uni Cluster as M90104 | |
| X57206 | 1D-myo-inositol-trisphosphate 3-kinase B (ITPKB) | + |
| U80040 | aconitase nuclear encoded mitochondrial prot | − |
| D86964 | dedicator of cytokinesis 1 homolog protein/KIAA0209 | + |
| U40714 | tyrosyl-tRNA synthetase (YARS) | + |
| X67235 | proline rich homeobox (Prh) prot; Also: L16499 | + |
| AC002045 | Nuclear pore complex-interacting protein (NPIP) | + |
| Z22548 | thiol-specific antioxidant peroxiredoxin 2 (PRDX2) | − |
| D28423 | pre-splicing factor SRp20 | + |
| S75578 | 4-aminobutyrate aminotransferase (ABAT) | − |
| HG3995- | Cpg-Enriched Dna Clone S19 | + |
| HT4265 | | |
| M24485 | glutathione S-transferase pi (GSTP1); Also: U21689 | − |
| M93405 | methylmalonate semialdehyde | + |
| | dehydrogenase (ALDH6A1) | |
| L36529 | (clone N5-4) protein p84 (THOC1) | − |
| L77561 | DiGeorge syndrome critical region 11 | + |
| | DGS-D (DGCR11) | |
| D83920 | uterus ficolin-1 | + |
| X63679 | TRAMP prot | + |
| D30758 | Centaurin beta 1 (CENTB1)/KIAA0050 | + |
| HG2715- | Tyrosine Kinase | + |
| HT2811 | | |
| D50925 | serine-threonine protein kinase/KIAA0135 | + |
| L42572 | p87/89 mitochondrial inner membrane protein (IMMT) | − |
| U25975 | serine kinase (hPAK65) | + |
| HG2479- | Helix-Loop-Helix prot Sef2-1d; Also: M74719 | + |
| HT2575 | | |
| X76648 | glutaredoxin | + |
| D82345 | NB thymosin beta | − |
| M13994 | bcl-2-alpha; Also: M14745 | + |
| D13639 | G1/S-specific cyclin D2/KIAK0002 | + |
| L39059 | TRANSCRIPTION FACTOR SL1 (TAF1C) | − |
| M81933 | Cell division cycle 25A (CDC25A) | + |
| X57809 | rearranged Ig lambda light chain | + |
| U41740 | trans-Golgi p230 (GOLGA4) | − |
| D83784 | C2 H2-type zinc finger protein/KIAA0198 | + |
| HG4272- | Hepatocyte Growth Factor Receptor | + |
| HT4542 | | |
| M34181 | testis-specific cAMP-dependent prot | + |
| | kinase (PRKACB) | |
| X79353 | XAP-4 GDP-dissociation inhibitor (GDI1) | − |
| U20938 | Lymphocyte dihydropyrimidine | + |
| | dehydrogenase (DPYD) | |
| M58285 | membrane-associated prot (HEM-1) | + |
| L15309 | zinc finger prot (ZNF 141) | + |
| J04444 | cytochrome c-1 (CYC1) | − |
| Y08265 | DAN26 prot; Also: U94836 | + |
| D50532 | macrophage lectin 2 (HML2) | + |
| U43030 | cardiotrophin-1 (CTF1) | + |
| AB000450 | Vaccinia related kinase 2 (VRK2) | + |
| U73377 | p66shc (SHC) | − |
| X62654 | ME491/CD63 antigen | − |
| HG3570- | prot Phosphatase Inhibitor Homolog | − |
| HT3773 | | |
| U78575 | phosphatidylinositol 4-phosphate 5-kinase | + |
| | alpha PIP5K1A | |
| M63573 | secreted cyclophilin-like prot (SCYLP) | − |
| D31767 | DAZ associated protein 2/KIAA0058 | + |
| X94563 | dbi/acbp/X94563 | + |
| D31846 | DRPLA | + |
| U20647 | zinc finger prot (ZNF151) | + |
| X89101 | Fas/APO1 (TNFRSF6) | + |
| U31903 | CREB-RP (CREBL1); Also: U89337_1, X98054 | + |
| HG315- | Beta-1-Glycoprot 11 Pregnancy-Specific (PSG11) | + |
| HT315 | | |
| X75252 | phosphatidylethanolamine binding prot | − |
| Z48042 | encoding GPI-anchored prot p137 | + |
| M20778 | alpha-3 (VI) collagen; Also: X52022 | + |
| M81118 | alcohol dehydrogenase chi polypeptide (ADH5) | − |
| X76105 | Death-associated protein (DAP1) | − |
| M16279 | MIC2 | − |
| D83779 | Hypothetical protein/KIAA0195 | + |
| U07231 | G-rich sequence factor-1 (GRSF-1) | − |
| M28713 | NADH-cytochrome b5 reductase (b5R) | − |
| X16609 | ankyrin (variant 2.1); Also: HG2737-HT2837 | − |
| D87434 | Hypothetical protein/KIAA0247 | + |
| Y08409 | spot14 | + |
| D87735 | ribosomal prot L14 | + |
| M35416 | GTP-binding prot (RALB) | + |
| S79219 | metastasis-associated; Also: X14608 | − |
| U59309 | fumarase precursor (FH) | − |
| D28588 | Sp2 transcription factor/KIAA0048 | + |
| X94910 | ERp31 prot | + |
| M34175 | beta adaptin | + |
| X71125 | glutamine cyclotransferase (QPCT) | + |
| AF006087 | Arp2/3 prot complex subunit p20-Arc (ARC20) | + |
| U43431 | DNA topoisomerase III alpha (TOP3A) | − |
| D38491 | Hypothetical protein/KIAA0117 | + |
| U30521 | P311 HUM-31 | − |
| U03634 | P47 LBC onco | + |
| M13955 | mesothelial keratin K7 (type II) | + |
| S78432 | un-named-transcript-1 from SAS = transmembrane 4 | − |
| K03218 | src sarcoma viral oncogene homolog (SRC) | + |
| L04270 | (clone CD18) tumor necrosis factor | − |
| | receptor 2 related prot | |
| U38291 | microtubule-associated prot 1a (MAP1A) | − |
| | genomic sequence | |
| U78027 | L44L (L44-like ribosomal prot) | + |
| U09303 | T cell leukemia LERK-2 (EPLG2)/Ephrin-B1 | + |
| AB002365 | Hypothetical protein/KIAA0367 | − |
| AD000092 | RAD23A homolog | + |
| U49395 | ionotropic ATP receptor P2X5a | + |
| U70732 | glutamate pyruvate transaminase (GPT) | + |
| U41068 | retinoid X receptor beta (RXRbeta)/collagen | + |
| | alpha2(XI) | |
| Z25884 | CIC-1 muscle chloride channel protein (CLCN1) | + |
| X65488 | Heterogeneous nuclear ribonucleoprotein U (HNRPU) | + |
| M63959 | alpha-2-macroglobulin receptor-associated prot | − |
| U14391 | myosin-IC | − |
| M10277 | cytoplasmic beta-actin; Also: HSAC07/X00351 | + |
| D25217 | Membrane protein MLC1/KIAA0027 | + |
| D87969 | CMP-sialic acid transporter (SLC35A1) | + |
| M83221 | I-Rel | + |
| M12759 | Ig J chain (IGJ) | + |
| X76534 | NMB | − |
| Z48923 | BMPR-II | + |
| M31520 | Ribosomal S24 | − |
| U15173 | Nip2 (NIP2) | + |
| AB002559 | hunc18b2 | + |
| U49837 | LIM prot MLP (CSRP3) | + |
| X02761 | fibronectin (FN precursor); Also: HG3044-HT2527 | − |
| L14754 | DNA-binding prot (SMBP2) | + |
| AF008937 | syntaxin-16C | + |
| U58032 | myotubularin related prot 1 (MTMR1)/U58032 | + |
| Z12962 | homolog to yeast ribosomal prot L41 | + |
| U88964 | HEM45 | + |
| X98534 | Vasodilator-stimulated phosphoprotein | + |
| | (VASP); Z46389 | |
| S75463 | P43 = mitochondrial elongation factor homolog | − |
| Y09980 | HOXD3 | − |
| U48936 | amiloride-sensitive epithelial sodium | + |
| | channel gamma subunit | |
| M15841 | U2 small nuclear RNA-associated B′ antigen | − |
| Z18951 | caveolin | − |
| D86965 | Hypothetical protein/KIAA0210 | + |
| L19058 | glutamate receptor (GLUR5) | + |
| U77594 | tazarotene-induced 2 (TIG2) | − |
| M55543 | guanylate binding prot isom II (GBP-2) | − |

TABLE 6
Least discriminatory genes for MS (regardless
of treatment with Avonex or not) vs. Healthy Donors

| | | |
|---|---|---|
| HG4018- | Opioid-Binding Cell Adhesion Molecule | + |
| HT4288 | | |
| X79683 | Z68155 and others | − |
| M29696 | interleukin-7 receptor (IL-7) | + |
| L16896 | zinc finger prot | − |
| Y00285 | insulin-like growth factor II receptor; Also: J03528 | − |
| HG4115- | Olfactory Receptor Or17-210 | + |
| HT4385 | | |
| L32977 | ubiquinol cytochrome c reductase Rieske iron-sulphur prot | − |
| M65290 | Interleukin 12p40 (IL12p40/IL12B) | + |
| U15131 | p126 (ST5) | − |
| L48513 | paraoxonase 2 (PON2) | − |
| M31642 | hypoxanthine phosphoribosyltransferase (HPRT) | − |
| M83088 | phosphoglucomutase 1 (PGM1) | − |
| U83410 | CUL-2 (cul-2) | + |
| U41804 | putative T1/ST2 receptor binding prot precursor | − |
| U57094 | small GTP-binding prot | + |
| U05040 | FUSE binding protein (FUBP1) | + |
| L20815 | S protein/corneodesmosin (CDSN) | + |
| X66142 | rod cGMP phosphodiesterase 6b (PDE6B); Also: S41458 | + |
| X53586 | integrin alpha 6 | + |
| L25286 | alpha-1 type XV collagen; Also: L25285 | − |
| J03764 | Plasminogen activator inhibitor 1 | + |
| Z38026 | FALL-39 peptide antibiotic | − |
| U27325 | thromboxane A2 receptor (TBXA2R); Also: D38081 | + |
| HG4747- | Nadh-Ubiquinone Oxidoreductase 51 Kda Subunit | + |
| HT5195 | | |
| L40395 | (clone S20iii15) | + |
| L40387 | thyroid receptor interactor TRIP14 (OASL) | + |
| M31210 | Endothelial differentiation protein (EDG-1) | + |
| X56741 | rab8 | + |
| Z84497 | from cosmid O14 on chrom 6 contains RING3, CpG Island | + |
| L76568 | S26 from excision and cross link repair prot (ERCC4)/L76568 | − |
| S77583 | HERVK10/HUMMTV reverse transcriptase homolog/S77583 | + |
| M64099 | gamma-glutamyl transpeptidase-related prot (GGT-Rel) | − |
| L35253 | p38 mitogen activated prot (MAP) kinase; Also: L35264 | + |
| S82362 | hRAR- beta 2 = retinoic-acid-receptor beta/M62303 | + |
| U00238 | glutamine PRPP amidotransferase (GPAT) | − |
| U79526 | orphan G-prot coupled receptor Dez isoform a | − |
| HG4114- | Olfactory Receptor Or17-209 | + |
| HT4384 | | |
| L31584 | G prot-coupled receptor (EBI1) | + |
| X07203 | CD20 receptor (S7) | + |
| Y07701 | aminopeptidase | + |
| X13839 | vascular smooth muscle alpha-actin | − |
| D28114 | myelin-associated oligodendrocytic basic protein (MOBP) | + |
| HG3994- | Cpg-Enriched Dna Clone S16 | + |
| HT4264 | | |
| X78520 | CLCN3 | − |
| AF006084 | Arp2/3 prot complex subunit p41-Arc (ARC41) | + |
| U33920 | clone lambda 5 semaphorin 3F (SEMA3F) | + |
| M81637 | Grancalcin (GCA) | + |
| X89267 | DNA uroporphyrinogen decarboxylase (UROD) | + |
| X82200 | Staf50 | + |
| Z70759 | mitochondrial 16S rRNA ()/Z70759 | + |
| X78925 | HZF2 zinc finger prot | + |
| Y00486 | adenine phosphoribosyltransferase (aprt) | + |
| X59871 | TCF-1 T cell factor 1 | + |
| U50743 | NaK-ATPase gamma subunit | + |
| U06454 | AMP-activated prot kinase (hAMPK) | + |
| X75593 | rab 13 | − |
| U68233 | farnesol receptor HRR-1 (HRR-1) | + |
| HG2602- | Succinate Dehydrogenase Flavoprotein (HSSUCCDH) | + |
| HT2698 | | |
| D25304 | Rac/Cdc42 guanine exchange factor/KIAA0006 | + |
| HG884- | Oncogene E6-Ap, Papillomavirus; Also: U84404 | − |
| HT884 | | |
| U40705 | telomeric repeat binding factor (TRF1) | − |
| D42046 | DNA replication helicase-like homolog/KIAA0083 | + |
| U63541 | expressed in HC/HCC livers and MolT-4 proliferating cells | + |
| HG417- | Cathepsin B; Also: L22569 | − |
| HT417 | | |
| HG4258- | Kinase Inhibitor P27kip1 Cyclin-Dependent | + |
| HT4528 | | |
| Y10936 | hypothetical prot downstream of DMPK and DMAHP | + |
| D28364 | annexin II (ANXA2) | + |
| D29833 | salivary proline rich peptide P-B | + |
| U10886 | Protein tyrosine phosphatase (PTPRJ) | + |
| M13699 | ceruloplasmin (CP) | + |
| U24183 | phosphofructokinase (PFKM); Also: HG1849-HT1878 | − |
| U43083 | G alpha-q (Gaq) | + |
| D83657 | calcium-binding prot in amniotic fluid 1 CAAF1 (S100A12) | + |
| Y13618 | DFFRY prot | + |
| U60060 | FEZ1 | − |
| U03399 | T-complex prot 10A (TCP10A) | + |
| X68836 | S-adenosylmethionine synthetase (MAT2A) | + |
| M81758 | voltage-dependent sodium channel SKM1 (SCN4A) | + |
| J02611 | apolipoprot D | + |
| M13577 | myelin basic prot (MBP) | + |
| M73077 | glucocorticoid receptor repression factor 1 (GRF-1) | − |
| X16983 | integrin alpha-4 subunit VLA4 (ITGA4) | + |
| AF002224 | E6-AP ubiquitin prot ligase 3A (UBE3A) | + |
| HG3638- | Amyloid Beta (A4) Precursor prot; Also: Y00264 | − |
| HT3849 | | |
| L07758 | IEF SSP 9502 | − |
| M87507 | interleukin-1 beta convertase (IL1BCE); Also: U13697 | + |
| Y00062 | T200 leukocyte common antigen (CD45 LC-A) | + |
| M81750 | myeloid cell nuclear differentiation antigen | + |
| X06825 | skeletal beta-tropomyosin | − |
| U07919 | aldehyde dehydrogenase 6 | + |
| D88613 | HGCMa/glial cells missing homolog 1 (GCM1) | + |
| U17969 | initiation factor eIF-5A | − |
| M31013 | nonmuscle myosin heavy chain (NMHC) | − |
| M57763 | ADP-ribosylation factor (hARF6) | + |
| X76061 | p130 retinoblastoma-like 2 (RBL2) | + |
| AF007111 | MDM2-like p53-binding prot (MDMX) | + |
| K02574 | purine nucleoside phosphorylase (PNP) | + |
| X90761 | Keratin, hair, acidic, 2 hHa2 (KRTHA2) | + |
| D49490 | disulfide isomerase-related protein (PDIR) | + |
| L12350 | thrombospondin 2 (THBS2) | − |
| X96506 | NC2 alpha subunit; Also: U41843 | + |
| L19183 | MAC30 | − |
| D86971 | Hypothetical protein/KIAA0217 | + |
| L42563 | (clone ISW34) non-gastric HK-ATPase (ATP1AL1) | + |
| D25538 | adenylate cyclase 7 (ADCY7)/KIAA0037 | + |
| L06633 | TRANSCRIPTION FACTORS | + |
| M77349 | transforming growth factor-beta induced product (BIGH3) | − |
| Z35093 | SURF-1 | − |
| L06845 | cysteinyl-tRNA synthetase | + |
| HG3627- | Calcium Channel Voltage-Gated Beta 1 Subunit L Type 2 | + |
| HT3836 | | |
| X55330 | aspartylglucosaminidase (AGA) | − |
| U75272 | gastricsin/progastricsin (PGC); Also: J04443 | + |
| J04162 | leukocyte IgG receptor (Fc-gamma-R) | + |
| Z29505 | nucleic acid binding prot sub23 | + |
| M86406 | skeletal muscle alpha 2 actinin (ACTN20 | − |
| U67963 | lysophospholipase homolog (HU-K5) | − |
| S69272 | 38 kda intracellular serine protase inhibitor; Also: Z22658 | − |
| S82297 | beta 2-microglobulin | + |
| S50017 | 2,3-cyclic nucleotide 3-phosphodiesterase (CNP) | + |
| K02777 | T-cell receptor active alpha-chain from Jurkat cell line/M12959 | + |
| M27749 | Ig-related 14.1 prot | + |
| M55621 | N-acetylglucosaminyltransferase I (GlcNAc-TI) | + |
| X52011 | MYF6 encoding a muscle determination factor | + |
| HG2815- | Myosin, Light Chain/U02629; Also: HG2815-HT1357, M22919 | − |
| HT2931 | | |
| HG3884- | Homeotic protein HPX42 | − |
| HT4154 | | |
| D87470 | hypothetical protein/KIAA0280 | − |
| D37781 | protein-tyrosine phosphatase (PTPRJ); Also: U10886 | + |
| U41766 | metalloprotease/disintegrin/cysteine-rich prot precursor MDC9 | − |
| Z11559 | iron regulatory factor | − |
| U34380 | prot tyrosine kinase TEC and TXK; Also: D29767 | + |
| X68486 | A2a adenosine receptor | + |
| M19267 | tropomyosin; Also: X12369 | − |
| M64497 | apolipoprot AI regulatory prot (ARP-1) | − |
| Z24727 | tropomyosin isoform | − |
| Y09836 | unknown prot | − |
| D80006 | Hypothetical protein/KIAA0184 | + |
| U78678 | thioredoxin 2 (TXN2) | + |
| HG4094- | Transcription Factor Lsf-Id; Also: U03494 | + |
| HT4364 | | |
| L10717 | T cell-specific tyrosine kinase | + |
| U22431 | hypoxia-inducible factor 1 alpha; X72726, U29165 | − |
| U07857 | 18 kDa Alu RNA binding prot | − |
| X97303 | Ptg-12 prot/X97303 | + |
| X98172 | MACH alpha 1 prot | + |
| L35240 | enigma | − |
| Z74616 | prepro-alpha2(l) collagen; Also: J03464 | − |
| D56495 | Reg-related sequence derived peptide-2 (REGL) | + |
| U52827 | Cri-du-chat region clone NIBB11 | + |
| HG1751- | CSH5 | + |
| HT1768 | | |
| Y10517 | CD108 prot/Y10517 | + |
| M37721 | peptidylglycine alpha-amidating monooxygenase | − |
| D87716 | hypothetical protein KIAA0007; Also: D26488 | − |
| U83908 | nuclear antigen H731 | + |
| D38024 | facioscapulohumeral muscular dystrophy (FSHD) | + |
| HG2755- | T-Plastin | − |
| HT2862 | | |
| U65579 | mitoch NADH dehydrogenase-ubiquinone Fe-S prot | − |
| U05237 | fetal Alz-50-reactive clone 1 (FAC1) | + |
| D12620 | cytochrome P-450LTBV; Also: U02388 | + |
| L10413 | farnesyltransferase alpha-subunit | − |
| D87445 | Hypothetical protein/KIAA0256 | + |
| U33822 | tax1-binding prot TXBP181, MAD1-like 1 (MAD1L1) | + |
| S81957 | BMP-5 = bone morphogenic prot-5/S81957 | + |
| L06499 | ribosomal prot L37a (RPL37A) | + |
| D86957 | Septin-like protein/KIAA0202 | − |
| U73477 | acidic nuclear phosphoprot pp32; Also: X75090 | + |
| M22632 | mitochondrial aspartate aminotransferase | − |
| X60188 | ERK1 prot serine/threonine kinase | − |
| AF001359 | mismatch repair prot (hMLH1)/AF001359 | − |
| Z68193 | Opsin 1 (OPN1LW) | + |
| U82311 | unknown prot/U82311 | + |
| U34044 | selenium donor prot (seID) | − |
| U55206 | gamma-glutamyl hydrolase (hGH) | − |
| U90916 | clone 23815 sequence, IFN-inducible | + |
| M16424 | beta-hexosaminidase alpha chain (HEXA) | + |
| U46025 | translation initiation factor elF-3 p110 subunit | − |
| M90391 | Interleukin 16 (IL16) | + |
| U49835 | YKL-39 precursor; Also: U58514, U58515 | + |
| HG2668- | Bradykinin Receptor | + |
| HT2764 | | |
| Z11793 | selenoprot P | − |
| X64364 | M6 antigen | − |
| M58028 | ubiquitin-activating enzyme E1 (UBE1) | − |
| X66839 | MaTu MN p54/58N carbonic anhydrase 9 protein (CA9) | + |
| L17327 | pre-T/NK cell associated prot (3B3) | + |
| X80692 | ERK3 | − |
| U09550 | oviductal glycoprotein (OVGP1) | + |
| X64330 | ATP-citrate lyase | − |
| U81006 | p76 transmembrane 9 superfamily member 2 (TM9SF2) | + |
| Y09943 | NGF-inducible PC3 anti-proliferative protein (BTG2) | + |
| U70862 | nuclear factor I/B (NFIB) | + |
| U24685 | anti-B cell autoantibody lgM heavy chain variable V-D-J region | + |
| HG3991- | Cpg-Enriched Dna, Clone E18 | + |
| HT4261 | | |
| 80343 | ArgRS = arginyl-tRNA synthetase | − |
| U37146 | silencing mediator of retinoid and thyroid hormone SMRT | − |
| U47101 | NifU-like prot (hNifU) | − |
| M13241 | N-myc | − |
| M24899 | triiodothyronine (ear7) | − |
| AB000896 | cadherin FIB2 | + |
| M19283 | cytoskeletal gamma-actin | − |
| L25085 | Sec61-complex beta-subunit | − |
| L07261 | alpha adducin (ADD1) | + |
| S65738 | actin depolymerizing factor | − |
| D14889 | small GTP-binding protein S10 (RAB33A) | + |
| U40462 | Ikaros/LyF-1 homolog (hlk-1) | + |
| M26730 | mitochondrial ubiquinone-binding prot; Also: X13585, M22348 | − |
| M28219 | low density lipoprot receptor (FH 10) | + |
| X52192 | RNA c-fes | + |
| L76571 | nuclear hormone receptor (shp) | − |
| M76378 | cysteine-rich prot (CRP) | − |
| AB006190 | aquaporin 6 (AQP6) | + |
| M54995 | connective tissue activation peptide III | + |
| U51561 | cosmid N79E2 sequence | + |
| U29680 | A1 prot | + |
| AB000584 | TGF-beta superfamily prot | + |
| HG3517- | Alpha-1-Antitrypsin | + |
| HT3711 | | |
| X52599 | beta nerve growth factor | + |
| U89922 | lymphotoxin beta isoform variant/L11016_rna1, L11015 | + |
| AB000897 | cadherin FIB3 | + |
| HG627- | Rhesus (Rh) Blood Group Ce-Antigen, 2, Rhvi; Also: X63097 | + |
| HT5097 | | |
| D89859 | zinc finger 5 protein (ZNF5) | + |
| X63422 | delta-subunit of mitochondrial F1F0 ATP-synthase (clone #1) | − |
| X98253 | ZNF183/X98253 | + |
| M37104 | mitochondrial ATPase coupling factor 6 subunit (ATP5A) | − |
| X57809 | rearranged Ig lambda light chain; Also: S42404 | + |
| M31899 | DNA repair helicase (ERCC3) | − |
| X16663 | HS1 heamatopoietic lineage cell specific prot | + |
| D86960 | Hypothetical protein/KIAA0205 | + |
| X06700 | pro-alpha1(III) collagen; Also: X14420 | − |
| X12794 | v-erbA related ear-2/NR2F6 | + |
| L10338 | sodium channel beta-1 subunit (SCN1B)/U12194, L16242 | + |
| L38935 | GT212 | + |
| U52101 | desmocollin-2 3 | + |
| U35113 | metastasis-associated mta1 | − |
| X02875 | (2-5 ) oligo A synthetase E (1,8 kb RNA); Also: M11810_2 | + |
| Z29083 | 5T4 Oncofetal antigen | − |
| X69141 | squalene synthase | − |
| D80004 | Hypothetical protein/KIAA0182 | + |
| M29994 | alpha-I spectrin (SPNA1); Also: M61877, M61826 | + |
| D42073 | reticulocalbin | − |
| X90846 | mixed lineage kinase 2 (MAP3K10) | + |
| L13329 | iduronate-2-sulfatase (IDS) | + |
| U09813 | mitochondrial ATP synthase subunit 9 P3 | − |
| M91029 | AMP deaminase (AMPD2) | + |
| HG2815- | HG2815-HT2931_at | − |
| HT2931 | | |
| Z78289 | (clone 1D2)/Z78289 | + |
| M23114 | calcium-ATPase (HK1) | − |
| U10686 | MAGE-11 antigen (MAGE11) | + |
| M15395 | leukocyte adhesion prot (LFA-1/Mac-1/p150,95 family) | + |
| M19045 | lysozyme | + |
| X96401 | MAX-binding ROX protein | + |
| M33336 | cAMP-dependent prot kinase type I-alpha subunit (PRKAR1A) | + |
| D13626 | G protein-coupled receptor/KIAA0001 | − |
| U71300 | snRNA activating prot complex 50 kD subunit (SNAP50) | + |
| J02854 | 20-kDa myosin light chain (MLC-2) | − |
| AJ000480 | C8FW phosphoprot | + |
| U40998 | retinal prot (HRG4) | + |
| HG3893- | Phosphoglucomutase 1 | + |
| HT4163 | | |
| X17567 | snRNP prot B; Also: J04564 | − |
| D42108 | phospholipase C-like 1 (PLCL1) | + |
| U79271 | clones 23920 and 23921 sequence | − |
| M94046 | zinc finger prot (MAZ) | − |
| X89067 | trpc2 transcript (possible pseudo) | + |
| X57398 | pM5 prot | − |
| HG2649- | Serine/Threonine prot Kinase Cdk3; Also: X66357 | − |
| HT2745 | | |
| U82535 | fatty acid amide hydrolase (FAAH) | + |
| X59798 | PRAD1 cyclin | − |
| HG2480- | Fmlp-Related Receptor I | + |
| HT2576 | | |
| U18009 | chromosome 17q21 clone LF113 | − |
| M64571 | microtubule-associated prot 4 | − |
| D82344 | NBPhox (PHOX2B) | + |
| X61587 | rhoG GTPase | + |
| M35531 | fucosyltransferase (FUT1) | + |
| HG4322- | Tubulin, Beta | − |
| HT4592 | | |
| M55210 | laminin B2 chain (LAMB2) | − |
| X95735 | zyxin 2 | − |
| L42176 | (clone 353) DRAL | − |
| X60036 | mitochondrial phosphate carrier prot | − |
| X57766 | stromelysin-3 | − |
| HG4683- | TNF Receptor 2 Associated prot Trap3/U12597 | − |
| HT5108 | | |
| L26339 | autoantigen | − |
| D38437 | DNA mismatch repair | − |
| S67798 | PH-20 | + |
| Z47727 | RNA polymerase II subunit | + |
| U68142 | RalGDS-like 2 (RGL2) | + |
| D87436 | Lipin 2/KIAA0249 | + |
| Z19554 | vimentin (VIM); Also: M18895_2 | − |
| J00123 | enkephalin | − |
| D42138 | Phosphatidylinositol glycan type B (PIG-B) | + |
| M65214 | (HeLa) helix-loop-helix prot HE47 (E2A); Also: M31523 | + |
| U57342 | myelodysplasia/myeloid leukemia factor 2 (MLF2) | − |
| D85245 | TR3beta | + |
| U39447 | placenta copper monamine oxidase | − |
| U07807 | metallothionein IV (MTIV) | + |
| U97018 | echinoderm microtubule-associated prot homolog HuEMAP | − |
| D87076 | Br140/KIAA0239 | + |
| U34252 | gamma-aminobutyraldehyde dehydrogenase | − |
| S72487 | Platelet-derived endothelial growth factor 1 (ECGF1) | + |
| J02963 | platelet glycoprot IIb | + |
| U30255 | phosphogluconate dehydrogenase (hPGDH) | + |
| D38524 | 5-nucleotidase (NT5C2) | + |
| U46751 | phosphotyrosine independent ligand p62 | − |
| HG1879- | Ras-Like prot Tc10 | + |
| HT1919 | | |
| L13434 | chromosome 3p211 sequence | + |
| U37012 | cleavage and polyadenylation specificity factor | + |
| HG4662- | Omega Light Chain Ig Lambda Light Chain Related | + |
| HT5075 | | |
| M29536 | translational initiation factor 2 beta subunit (eIF-2-beta) | − |
| D21235 | HHR23A prot | − |
| U65093 | msg1-related 1 (mrg1) | + |
| M32011 | neutrophil oxidase factor (p67-phox) | + |
| X04412 | plasma gelsolin | − |
| S80905 | PRB2 (PRB2L CON1+) = Con1 | + |
| M60626 | N-formylpeptide receptor 1 (FPR1) | + |
| D83243 | NPAT | + |
| X73478 | hPTPA | − |
| J05243 | nonerythroid alpha-spectrin (SPTAN1) | − |
| S42303 | N-cadherin | − |
| Y09305 | prot kinase Dyrk4 | − |
| HG1980- | Tubulin, Beta 2 | − |
| HT2023 | | |
| L42611 | keratin 6 isoform K6e (KRT6E) | + |
| U66619 | SWI/SNF complex 60 KDa subunit (BAF60c) | − |
| U40282 | integrin-linked kinase (ILK) | − |
| AB004884 | PKU-alpha | + |
| X72841 | IEF 7442 | − |
| U13695 | homolog of yeast mutL (hPMS1) | + |
| U67171 | selenoprot W (selW)/U67171 | − |
| X97335 | kinase A anchor prot | − |
| U82130 | tumor susceptiblity prot (TSG101) | − |
| Z80780 | H2B/h/Z80780 | + |
| U52112 | Renin binding protein (RENBP) | + |
| U14193 | TFIIA gamma subunit | − |
| U66618 | SWI/SNF complex 60 KDa subunit (BAF60b) | + |
| M24461 | pulmonary surfactant-associated prot SP-B (SFTP3) | + |
| U77735 | pim-2 protoonco homolog pim-2 h | + |
| D25216 | hypothetical protein/KIAA0014 | − |
| U28369 | semaphorin V | − |
| J04611 | lupus p70 (Ku) autoantigen prot | − |
| X76770 | PAP | + |
| U58970 | outer mitochondrial membrane translocase (TOMM34) | − |
| U78628 | leukemia inhibitory factor receptor/U78628 | + |
| Z68274 | cosmid L129H7, Huntingtons Disease Region/Z68274 | + |
| X13967 | leukaemia inhibitory factor (LIF/HILDA) | + |
| L37868 | POU-domain transcription factor (N-Oct-3); Also: Z11933 | − |
| U35451 | heterochromatin prot p25 | − |
| X15880 | collagen VI alpha-1 C-terminal globular domain | − |
| X79780 | YPT3 | − |
| D63485 | Inhibitor of NFkB kinase epsilon subU. IkBKE/KIAA0151 | + |
| HG3748- | Basic Transcription Factor 44 Kda Subunit | + |
| HT4018 | | |
| X80907 | phosphatidyl-inositol-3-kinase p85 (PIK3R2) | + |
| U31930 | deoxyuridine nucleotidohydrolase | − |
| U93049 | SLP-76 associated prot | + |
| M74715 | alpha-L-iduronidase (IDUA); Also: M95740 | + |
| D82061 | short-chain alcohol dehydrogenase family | + |
| L10838 | SR prot family pre-splicing factor (SRp20) | + |
| X55448 | Glucose 6 phosphate dehydrogenase | − |
| U82979 | Ig-like transcript-3 (LILRB4) | + |
| M21154 | S-adenosylmethionine decarboxylase | + |
| U51432 | nuclear prot Skip | − |
| X54870 | NKG2-D | + |
| D87440 | hypothetical protein/KIAA0252 | − |
| X85116 | epb72; Also: U33931 Same Unigene Cluster as M81635 | − |
| Z23090 | 28 kDa heat shock prot | − |
| HG2992- | Beta-Hexosaminidase Alpha Polypeptide | + |
| HT5186 | | |
| M55593 | collagenase type IV (CLG4) | − |
| X15341 | COX VIa-L cytochrome c oxidase liver-specific subunit Via | − |
| HG4638- | Spliceosomal prot Sap 49 | + |
| HT5050 | | |
| U59058 | beta-A3/A1 crystallin (CYRBA3/A1); Also: M14306 | + |
| HG3255- | Gaba A Receptor Beta 2 Subunit | + |
| HT3432 | | |
| X83368 | phosphatidylinositol 3 kinase gamma (PIK3CG) | + |
| L46720 | autotaxin-t (atx-t); Also: L35594 | − |
| J03040 | SPARC/osteonectin | − |
| HG3214- | Metallopanstimulin 1 | + |
| HT3391 | | |
| HG4102- | N-Ethylmaleimide-Sensitive Factor (NSF) | + |
| HT4372 | | |
| U79295 | clone 23961 sequence | + |
| M54915 | h-pim-1 prot (h-pim-1); Also: M27903, M24779 | + |
| L13848 | RNA helicase A (DDX9) | + |
| M86528 | neurotrophin-4 (NT-4) | + |
| M96843 | striated muscle contraction regulatory prot (Id2B) | + |
| J02883 | Colipase (CLPS) | + |
| HG1602- | Utrophin | + |
| HT1602 | | |
| L41559 | pterin-4a-carbinolamine dehydratase (PCBD) | − |
| X01677 | glyceraldehyde-3-phosphate dehydrogenase | − |
| M80899 | novel protein desmoyokin (AHNAK) | + |
| D50915 | hypothetical protein/KIAA0125 | + |
| D49728 | NAK1 DNA binding prot, | + |
| U82671 | HSP1-A from cosmids from Xq28 | + |
| S45630 | alpha B-crystallin = Glioma Rosenthal fiber component | − |
| AB002318 | talin homolog/KIAA0320 | + |
| L42373 | prot phosphatase 2A B56-alpha | + |
| L10284 | integral membrane prot calnexin (IP90) | − |
| M81780 | Sphingomyelin phosphodiesterase 1 (SMPD1) | + |
| M91036 | G-gamma globin (HBG2) | + |
| Z24725 | mitogen inducible mig-2 | − |
| D29805 | beta-14-galactosyltransferase | + |
| HG358- | Homeotic prot 7 Notch Group | + |
| HT358 | | |
| Z50022 | surface glycoprot | − |
| X99664 | prot containing SH3 domain SH3GL3 | + |
| AB002380 | leukemia-associated Rho GEF/KIAA0382 | − |
| X63131 | My1 (PML); Also: M73778 | − |
| J00129 | fibrinogen beta-chain | + |
| S62539 | insulin receptor substrate-1 | − |
| X51956 | ENO2 neuron specific (gamma) enolase | − |
| M22382 | mitochondrial matrix prot P1 (nuclear encoded) | − |
| S68874 | EP3 prostanoid receptor EP3-I/D86096_1/X83858/L27490 | + |
| D29954 | hypothetical protein/KIAA0056 | + |
| U70735 | 34 kDa mov34 isologue/U70735 | − |
| X69878 | Flt4 transmembrane tyrosine kinase | + |
| HG36- | PM-Sc1 autoantigen/M58460 | − |
| HT4101 | | |
| L24774 | delta3, delta2-CoA-isomerase; Also: Z25821_rna1, Z25820 | − |
| L04953 | Amyloid precursor protein binding APBA1 | + |
| X77548 | RFG | + |
| U18919 | chromosome 17q12-21 clone pOV-2 | − |
| S68805 | L-arginine: glycine amidinotransferase | − |
| M62782 | insulin-like growth factor binding prot 5 (IGFBP-5)/M65062 | − |
| Z97074 | Rab9 effector p40/Z97074 | − |
| AB002315 | Hypothetical protein/KIAA0317 | + |
| U22398 | Cdk-inhibitor p57KIP2 (KIP2) | − |
| M23197 | differentiation antigen (CD33) | + |
| U01147 | guanine nucleotide regulatory prot (ABR) | − |
| U51586 | siah binding prot 1 (SiahBP1) | − |
| M83652 | complement component properdin; Also: X57748, X70872_rna1 | + |
| X04143 | bone gla prot (BGP) | − |
| M13928 | X64467_rna1 and others | − |
| U15642 | transcription factor E2F-5; Also: U31556 | + |
| U18235 | ATP-binding cassette prot (ABC2) HFBCD04 clone | + |
| U84487 | CX3C chemokine precursor | − |
| X95404 | non-muscle type cofilin | − |
| D63160 | DNA lectin P35/Ficolin 2 (FCN2) | + |
| X13255 | dopamine beta-hydroxylase type a (EC 114171) | + |
| M12125 | fibroblast muscle-type tropomyosin, | − |
| X99268 | B-HLH DNA binding prot | − |
| U65533 | regulator of nonsense transcript stability (RENT1)/D86988 | − |
| L22342 | nuclear phosphoprot | + |
| U00928 | clone CE29 4.1 (CAC)n/(GTG)n repeat-containing | + |
| M10901 | glucocorticoid receptor alpha | + |
| HG3942-HT4212 | Interferon | − |
| M27161 | MHC class I CD8 alpha-chain (Leu-2/T8) | + |
| D90276 | CGM7 nonspecific cross-reacting antigen (NCA) | + |
| U42359 | N33 prot/U42359 | − |
| HG987- | Mac25 | − |
| HT987 | | |
| L40933 | phosphoglucomutase-related prot (PGMRP) | − |
| M19961 | cytochrome c oxidase subunit Vb (coxVb) | − |
| X58199 | adducin 2 beta (ADD2); Also: S81083_1 | + |
| U20582 | actin-like peptide (LOC81569) | + |
| D30655 | eukaryotic initiation factor 4All | + |
| L10910 | splicing factor (CC13) | − |
| M11749 | Thy-1 glycoprot | − |
| D79989 | centaurin gamma-1/KIAA0167 | + |
| U12595 | tumor necrosis factor type 1 receptor associated prot (TRAP1) | − |
| L37042 | casein kinase I alpha isom (CSNK1A1) | + |
| X86809 | major astrocytic phosphoprot PEA-15 | − |
| M24766 | alpha-2 collagen type IV (COL4A2); Also: X05610 | − |
| HG1140- | Collagen, Type Vi, Alpha 2; Also: M34570 | − |
| HT4817 | | |
| X64624 | RDC-1 POU domain containing prot; Also: L20433 | + |
| D28473 | T-lymphocyte isoleucyl-tRNA synthetase; Also: U04953 | − |
| M29277 | isolate JuSo MUC18 glycoprot/M28882, X68264_rna1 | − |
| HG429- | B-Cell Growth Factor 1 | + |
| HT429 | | |
| L02840 | potassium channel Kv21 (KCNB1) | + |
| M33308 | Vinculin (VCL) | − |
| M22995 | ras-related prot (Krev-1) | + |
| X99226 | FAA prot | − |
| HG3104- | Serine Protease Met1 | + |
| HT3280 | | |
| U51010 | nicotinamide N-methyltransferase 1 and 5 ing region/U08021 | − |
| U32114 | caveolin-2 | − |
| M60750 | histone H2B.1 (H2B)/M60750 | + |
| HG3740- | Basic Transcription Factor 2 34 Kda Subunit | + |
| HT4010 | | |
| M24398 | parathymosin | − |
| Z11502 | annexin A13 (ANXA13) | + |
| J04080 | complement component C1r | − |
| U47927 | isopeptidase T (ISOT) | − |
| HG1800- | Ribosomal prot S20 | + |
| HT1823 | | |
| L10405 | DNA binding prot surfactant prot B/L10405 | − |
| D17400 | 6-pyruvoyl-tetrahydropterin synthase | − |
| J03798 | autoantigen small nuclear ribonucleoprot Sm-D | + |
| X99586 | SMT3C prot; Also: U67122, U83117, U61397 | + |
| D49487 | obese/Leptin (LEP); Also: U43653 | + |
| D29642 | GTPase/KIAA0053 | + |
| HG1155- | Colony-Stimulating Factor 1 Macrophage (CSF1) | + |
| HT4822 | | |
| X14046 | leukocyte antigen CD37 | + |
| U11690 | faciogenital dysplasia (FGD1) | − |
| U61234 | tubulin-folding cofactor C | + |
| U91932 | AP-3 complex sigma 3A subunit (AP3S1) | − |
| S81083 | beta subunit 63 kDa | − |
| D16593 | BDR-2 hippocalcin | − |
| M22760 | nuclear-encoded mitochondrial cytochrome c oxidase Va | − |
| U71364 | serine protase inhibitor (P19) | + |
| J03805 | phosphatase 2A; Also: M60484_rna1 | − |
| L10955 | carbonic anhydrase IV; Also: M83670 | + |
| X53416 | actin-binding prot (filamin) (ABP-280) | − |
| D42040 | RING3 protein/KIAA9001; Also: X62083, M80613 | + |
| X52221 | ERCC2 | + |
| M32334 | intercellular adhesion molecule 2 (ICAM-2) | + |
| X64559 | tetranectin | − |
| M16750 | pim-1 oncogene; Also: M27903, M24779, M54915 | + |
| M30269 | nidogen | + |
| M33374 | cell adhesion prot (SQM1) | − |
| M27396 | asparagine synthetase; Also: M15798 | − |
| M31627 | X box binding prot-1 (XBP-1) | + |
| X97230 | NK receptor, clone library 4M1#6 | + |
| U89012 | dentin matrix acidic phosphoprot 1 (DMP1) | + |
| D83597 | RP105 | + |
| M13207 | granulocyte-macrophage colony-stimulating factor (CSF1) | − |
| L04733 | kinesin light chain | − |
| X82240 | T cell leukemia/lymphoma 1 (TCL1A) | + |
| U65406 | KCNJ1 (from potassium channel ROM-K1-6) | + |
| U79263 | clone 23760 | + |
| S57212 | hMEF2C = myocyte enhancer-binding factor 2; Also: L08895 | + |
| X13461 | calmodulin-like prot (CLP); Also: M58026 | + |
| X12447 | aldolase A (ALDOA) (EC 41213) | − |
| M27504 | topoisomerase type II (Topo II)/M27504/Also: Z15115 | + |
| X54941 | ckshs1 Cks1 prot homolog | − |
| M28882 | MUC18 glycoprot; Also: X68264_rna1 | − |
| X93499 | RAB7 prot | + |
| Z49989 | smoothelin | − |
| U61500 | GT334 prot (GT334) | + |
| U38980 | PMS2 related (hPMSR6) | + |
| Z18954 | S100D calcium binding prot | + |
| U57627 | fetal brain oculocerebrorenal syndrome (OCRL1) | − |
| M64673 | heat shock factor 1 (TCF5) | − |
| U82279 | Ig-like transcript 2 (LILRB1) | + |
| M57609 | DNA-binding prot (GLI3) | + |
| U89335 | Notch 4 | + |
| M61733 | erythroid membrane protein 41 (EPB41) | + |
| Y07604 | nucleoside-diphosphate kinase | − |
| M37766 | MEM-102 glycoprot | + |
| D13634 | Hypothetical protein/KIAA0009 | + |
| X52142 | CTP synthetase (EC 6342) | − |
| D26018 | DNA polymerase delta subunit 3/KIAA0039 | − |
| X98743 | RNA helicase (Myc-regulated dead box prot) | + |
| X75535 | PxF prot | − |
| U27193 | prot-tyrosine phosphatase | − |
| U47686 | signal transducer and activator of transcription Stat5B/U48730 | + |
| L43964 | (clone F-T03796) STM-2 | − |
| U08316 | insulin-stimulated prot kinase 1 (ISPK-1) | + |
| D50683 | TGF-betallR alpha | + |
| L34355 | (clone p4) 50 kD dystrophin-associated glycoprot, | − |
| L24470 | prostanoid FP receptor | + |
| AC002464 | BAC clone RG331P03/AC002464 | − |
| L10678 | profilin II | − |
| X80590 | PHKG1 | + |
| U04270 | putative potassium channel subunit (KCNH2) | − |
| K02054 | gastrin-releasing peptide | + |
| M11313 | alpha-2-macroglobulin; Also: M36501 | − |
| HG4321- | Ahnak-Related prot | + |
| HT4591 | | |
| X15882 | collagen VI alpha-2 C-terminal globular domain | − |
| U50534 | BRCA2 region sequence CG003 | − |
| U94586 | NADH: ubiquinone oxidoreductase MLRQ subunit | − |
| M26576 | COL4A1 (alpha-1 type IV collagen) | − |
| U50079 | histone deacetylase HD1; Also: D50405 | + |
| L38593 | D50402 and others | + |
| D26067 | Hypothetical protein/KIAA0033 | + |
| X13589 | aromatase (estrogen synthetase) (CYP19A1) | + |
| L13689 | prot-onco (BMI-1) | − |
| X80909 | alpha NAC nascent polypeptide-associated complex | − |
| Y11710 | extracellular matrix prot collagen type XIV, C-terminus | − |
| D38550 | E2f-3 transcription factor/KIAA0075 | + |
| M65062 | insulin-like growth factor binding prot 5 (IGFBP-5) | − |
| X74330 | DNA primase (PRIM1) | + |
| HG2743- | Caldesmon 1, 4, Non-Muscle; Also: HG2743-HT2843 | − |
| HT2846 | | |
| X94628 | MeCP-2; Also: L37298, Y12643, X89430 | − |
| U29953 | pigment epithelium-derived factor | − |
| D63879 | T cell-recognized SART-3/KIAA0156 | + |
| X81003 | HCG V | + |
| X60483 | H4/D histone | + |
| X92896 | ITBA2 prot | − |
| M73548 | polyposis locus (DP25) | + |
| M14058 | complement C1r | − |
| Z27113 | RNA polymerase II subunit 144 kD | − |
| J02923 | 65-kilodalton phosphoprot (p65) | + |
| X79568 | BDP1 prot-tyrosine-phosphatase | − |
| J03060 | glucocerebrosidase (GCB) | − |
| X07618 | cytochrome P450 db1 variant a; Also: X07619, X16866 | + |
| U55936 | SNAP-23 | + |
| U45328 | ubiquitin-conjugating enzyme (UBE2I); Also: U31882 | + |
| M17885 | acidic ribosomal phosphoprot P0 | − |
| U56402 | chromatin structural prot homolog (SUPT5H); Also: Y12790 | − |
| HG3431- | Decorin; Also: HG3431-HT3617 | − |
| HT3616 | | |
| X78338 | Synthetic adenovirus transformed retinal cell line MRP | + |
| J04621 | heparan sulfate proteoglycan (HSPG) core prot end | − |
| X74801 | Cctg chaperonin | − |
| U28055 | hepatocyte growth factor-like prot homolog (D1F15S1A) | − |
| M62810 | mitochondrial TRANSCRIPTION FACTORS 1 | − |
| HG4263- | Nkr-P1a prot | + |
| HT4533 | | |
| U52154 | G prot-coupled inwardly rectifying potassium channel Kir34 | + |
| U37352 | prot phosphatase 2AB alpha 1 | + |
| D50926 | Hypothetical protein/KIAA0136 | + |
| X16560 | COX VIIc subunit VIIc of cytochrome c oxidase (EC 1931) | − |
| U18088 | 3,5-cyclic AMP phosphodiesterase; Also: M37744, S75213 | − |
| L76159 | FRG1 | − |
| S90469 | cytochrome P450 reductase | − |
| HG3635- | Zinc Finger prot, Kruppel-Like | + |
| HT3845 | | |
| X69433 | mitochondrial isocitrate dehydrogenase (NADP+) | − |
| U09953 | ribosomal prot L9 | − |
| X06745 | DNA polymerase alpha-subunit (POLA) | − |
| HG3033- | Spliceosomal prot Sap 62 | + |
| HT3194 | | |
| D13720 | LYK; Also: L10717 | + |
| X60221 | H+-ATP synthase subunit b | − |
| M55905 | mitochondrial NAD(P)+ dependent malic enzyme | + |
| HG4128- | Anion Exchanger 3 Cardiac Isom | + |
| HT4398 | | |
| U62325 | FE65-like prot (hFE65L) | − |
| M21186 | neutrophil cytochrome b light chain p22 | + |
| U09851 | zinc finger prot (ZNF148); Also: L04282 | + |
| D50919 | TRIM14 protein/KIAA0129 | + |
| HG3044- | Fibronectin; Also: HG3044-HT2527, X02761 | − |
| HT3742 | | |
| M14676 | src-like kinase (slk) | + |
| D87673 | heat shock transcription factor 4 (HSF4) | + |
| J04809 | Cytosolic adenylate kinase AK1 | + |
| X15183 | 90-kDa heat-shock prot | − |
| M95623 | PBGD from hydroxymethylbilane synthase | − |
| U15174 | Nip3 (NIP3) | + |
| Z50115 | thimet oligopeptidase (metalloproteinase); Also: U29366 | − |
| AF001548 | 815A9.1 myosin heavy chain from chromosome 16 | − |
| M30894 | T-cell receptor Ti rearranged gamma-chain V-J-C region | + |
| L10615 | beta casein (CSN2); Also: X17070 | + |
| L14922 | DNA-binding prot (PO-GA) | + |
| U27768 | RGP4 | + |
| U68485 | Box-dependent MYC-interacting prot-1 (BIN1) | + |
| X14008 | lysozyme (EC 3.2.1.17) | + |
| X82693 | Lymphocyte antigen 6 complex, locus D (E48) | + |
| J05582 | Mucin 1 (MUC1); Also: J05581 | − |
| U77665 | RNaseP prot p30 (RPP30) | − |
| L05425 | autoantigen | − |
| U34343 | 13 kD differentiation-associated protein (DAP13) | − |
| D63486 | hypothetical protein/KIAA0152 | − |
| L32179 | arylacetamide deacetylase | − |
| D13628 | angiopoietin 1/KIAA0003 | − |
| U45973 | phosphatidylinositol (45)bisphosphate 5-phosphatase homolog | + |
| X05610 | type IV collagen alpha-2 chain | − |
| M94172 | N-type calcium channel alpha-1 subunit (CACNA1B) | + |
| Y08319 | kinesin-2 (KIF2) | + |
| S82447 | GCN5-like 1 = GCN5; Also: D64007 | − |
| U72512 | B-cell receptor associated prot (hBAP)/U72512 | + |
| X17648 | granulocyte-macrophage colony-stimulating factor receptor | + |
| L07044 | Ca-Calmodulin-dependent prot kinase CAMK | − |
| L06132 | voltage-dependent anion channel isom 1 (VDAC) | − |
| M95787 | 22 kDa smooth muscle prot (SM22) | − |
| U48296 | prot tyrosine phosphatase PTPCAAX1 (hPTPCAAX1) | − |
| M11718 | alpha-2 type V collagen | − |
| U24576 | breast tumor autoantigen sequence | − |
| X77383 | cathepsin-O | + |
| U50939 | amyloid precursor prot-binding prot 1 | − |
| U91903 | Fritz | − |
| U57341 | neurofilament L (NFL) | + |
| D83407 | ZAKI-4 | − |
| Z83735 | hH3/k | + |
| U70439 | silver-stainable prot SSP29; Also: Y07570 | − |
| D13435 | phosphatidyl-inositol-glycan class F | + |
| M67468 | Fragile X mental retardation 1 FMR-1; Also: X69962, L19493 | + |
| L32832 | alpha fetoprotein enhancer binding protein/D10250 | − |
| X59372 | HOX4C a homeobox prot | − |
| HG1103- | Guanine Nucleotide-Binding prot Ral | + |
| HT1103 | | |
| Z80345 | SCAD; Also: M26393 | − |
| U23946 | putative tumor suppressor (LUCA15) | + |
| D50810 | placental leucine aminopeptidase | + |
| L35475 | olfactory receptor-like | + |
| U07563 | ABL 1b and intron 1b, and putative M8604 Met prot | − |
| L19779 | histone H2A2 | + |
| M28825 | thymocyte antigen CD1a | + |
| D50918 | septin 2, 6 (SEPT6)/KIAA0128 | + |
| D13969 | Mel-18 prot | − |
| Z84718 | 22q11.2-qter DNA contains GSTT1-2 glutathione transferases | − |
| X53296 | IRAP; Also: X64532_rna1, X52015 | + |
| M63262 | 5-lipoxygenase activating prot (FLAP) | + |
| HG3546- | Pre- Splicing Factor Sf2p33; Also: M72709, M72709_rna2 | + |
| HT3744 | | |
| U72342 | platelet activating factor acetylhydrolase 45 kDa subunit (LIS1) | − |
| X51804 | PMI a putative receptor prot | − |
| L42243 | IFNAR2 (interferon receptor) | + |
| U09087 | thymopoietin (TMPO); Also: U09088 | + |
| J04617 | elongation factor EF-1-alpha; Also: M27364 | + |
| X54871 | ras-related prot RAB5B | + |
| M31520 | ribosomal prot S24; Also: HG3214-HT3391 | − |
| U90878 | LIM domain prot CLP-36 | − |
| X61373 | alternatively spliced tau/X61373 | + |
| L20860 | glycoprotein Ib beta (GP1BB) | + |
| D45371 | GS3109 adipose most abundant gene transcript 1 (APM1) | + |
| X16546 | eosinophil derived neurotoxin | + |
| M14636 | liver glycogen phosphorylase | + |
| X02176 | complement component C9; Also: K02766 | − |
| Z37166 | BAT1 nuclear RNA helicase (DEAD family) | − |
| L10844 | cellular growth-regulating prot | − |
| X56692 | C-reactive prot | + |
| L29376 | (clone 38-1) MHC class I fragment | + |
| U67932 | cAMP phosphodiesterase (Pde7A2)/U67932; Also: L12052 | + |
| M64595 | small G prot (Gx) | + |
| J02783 | thyroid hormone binding prot (p55) | − |
| D16181 | PMP2 | + |
| X72012 | endoglin | − |
| M14123 | HERV-K10 neutral protease | − |
| J03278 | MYCL2 | − |
| Y07827 | butyrophilin (BTN)/U90552 | + |
| S77812 | flt = vascular endothelial growth factor receptor/VEGF receptor | + |
| D12775 | erythrocyte-specific AMP deaminase; Also: U29926_2 | + |
| X95073 | translin associated prot X | + |
| D79999 | VPARP vault protein/KIAA0177 | + |
| M68891 | GATA-binding prot (GATA2) | + |
| U05321 | X-linked PEST-containing transporter (XPCT) | − |
| X80818 | metabotropic glutamate receptor type 4 | + |
| U20350 | G prot-coupled receptor V28 | + |
| M96233 | glutathione transferase class mu number 4 (GSTM4) | − |
| U19495 | intercrine-alpha (hIRH); Also: L36034 | − |
| J04101 | NAD(P)H: menadione oxidoreductase; Also: M81600 | + |
| L08177 | EBV induced G-prot coupled receptor (EBI2) | + |
| U40490 | nicotinamide nucleotide transhydrogenase | + |
| D13637 | toll-like receptor 1 (TLR1)/KIAA0012 | + |
| HG4704- | Glial Growth Factor 2 | + |
| HT5146 | | |
| U40571 | alpha1-syntrophin (SNT A1) | − |
| L37043 | casein kinase I epsilon | − |
| M28879 | granzyme B/CTLA-1 (GZMB) | + |
| U78190 | GTP cyclohydrolase I feedback regulatory prot | + |
| M61764 | gamma-tubulin | − |
| M81830 | somatostatin receptor isom 2 (SSTR2) | + |
| M83751 | arginine-rich prot (ARP) | − |
| D25218 | yeast ribosome biogenesis regulator homolog/KIAA0112 | + |
| L10377 | (clone CTG-B37) sequence; Also: D38529, U23851, D31840 | − |
| U80457 | TRANSCRIPTION FACTORS SIM2 short form | − |
| Z50194 | PQ-rich prot | + |
| X51405 | carboxypeptidase E (EC 341710) | − |
| Y08999 | Sop2p-like prot | − |
| M16447 | dihydropteridine reductase (hDHPR) | + |
| L20316 | glucagon receptor | + |
| D78132 | Ras homolog enriched in brain (RHEB); Also: Z29677 | − |
| S71129 | acetylcholinesterase (ACHE) | + |
| U79301 | clone 23842 sequence | + |
| D14661 | Splicing regulator WTAP protein/KIAA0105 | + |

TABLE 7
Highly discriminatory genes for MS (on no treatment)
vs. Healthy Donors and ALS

| | | Up (+) or down |
|---|---|---|
| Probe sets | Gene Descriptions | (−) |
| D30037 | phosphatidylinositol transfer protein (PITPN) | + |
| D29675 | iNOS | + |
| Z25884 | CIC-1 muscle chloride channel protein (CLCN1) | + |
| U78095 | Placental bikunin (AMBP) | + |
| U14187 | receptor tyrosine kinase ligand LERK-3/Ephrin-A3 | + |
| L34357 | GATA-4 | + |
| U79528 | SR31747 binding prot 1 (SRBP1); Also: U75283 | + |
| HG3991-HT4261 | Cpg-Enriched Dna, Clone E18 | + |
| U18271 | thymopoietin (TMPO) | + |
| HG2415-HT2511 | Transcription Factor E2f-2 | + |
| Z16411 | phospholipase c; Also: U26425, Z37544 (PLCB3) | − |
| D26069 | Centaurin beta 2 (CENTB2)/KIAA0041 | + |
| D45132 | kidney zinc-finger DNA-binding protein PRDM2 | + |
| D87450 | parallel sister chromatids drosophila prot-like/KIAA0261 | + |
| M25269 | tyrosine kinase (ELK1) onco | + |
| D38491 | Hypothetical protein/KIAA0117 | + |
| X72879 | 14A2AK DNA sequence | + |
| HG2825-HT2949 | Ret Transforming | + |
| M13994 | bcl-2-alpha; Also: M14745 | + |
| Z15114 | protein kinase C gamma (PRKCG) | + |
| U03398 | receptor 4-1BB ligand (TNFSF9) | + |
| D38449 | G protein-coupled receptor (GPR) | + |
| HG3175-HT3352 | Carcinoembryonic Antigen | + |
| M19650 | 2,3-cyclic nucleotide 3-phosphodiesterase (CNP) | + |
| M30818 | interferon-induced cellular resistance mediator MxB (MX2) | + |
| X65977 | corticostatin HP-4 precursor (defensin/DEFA4) | + |
| S68874 | EP3 prostanoid receptor EP3-I | + |
| U57341 | neurofilament L (NFL) | + |
| L05512 | histatin 1 (HTN1) | + |
| X83492 | Fas/APO1 (TNFRSF6) | + |
| D64158 | cell differentiation-associated ATP binding prot | + |
| D30036 | phosphatidylinositol transfer protein (PITPN) | + |
| X80878 | R kappa B (NFRKB) | + |
| U66059 | TCRBV1S1A1N1 from germline TCR beta chain | + |
| U39318 | E2 ubiquitin conjugating enzyme UbcH5C (UBCH5C) | + |
| U59752 | Sec7p-like prot | + |
| L34075 | FKBP-rapamycin associated prot (FRAP) | + |
| HG4108-HT4378 | Olfactory Receptor Or17-24 | + |
| X07619 | cytochrome P450 db1 variant b; Also: X16866 | + |
| HG3730-HT4000 | Tyrosine Kinase Syk; Also: L28824 | + |
| M35416 | GTP-binding prot (RALB) | + |
| M13577 | myelin basic prot (MBP) | + |
| S76617 | protein tyrosine kinase (BLK) | + |
| L36922 | Met-ase 1/Granzyme M (GZMM) | + |
| D49487 | obese/Leptin (LEP); Also: U43653 | + |
| D83784 | C2 H2-type zinc finger protein/KIAA0198 | + |
| L20860 | glycoprotein lb beta (GP1BB) | + |
| U61500 | GT334 prot (GT334) | + |
| M60626 | N-formylpeptide receptor 1 (FPR1) | + |
| D79993 | Hypothetical/KIAA0171/Enthoprotin (ENTH) | + |
| U22028 | cytochrome P450 (CYP2A13) | + |
| U63717 | osteoclast stimulating factor (OSTF1) | + |
| Z46632 | HSPDE4C1 3,5-cyclic AMP phosphodiesterase (PDE4C) | + |
| L77561 | DiGeorge syndrome critical region 11 DGS-D (DGCR11) | + |
| M34181 | testis-specific cAMP-dependent prot kinase (PRKACB) | + |
| S82362 | hRAR-beta 2 = retinoic-acid-receptor beta | + |
| U50743 | NaK-ATPase gamma subunit | + |
| D26579 | transmembrane prot ADAM8 | + |
| U20647 | zinc finger prot (ZNF151) | + |
| HG4094-HT4364 | Transcription Factor Lsf-Id; Also: U03494 | + |
| U34301 | nonmuscle myosin heavy chain IIB | + |
| U53347 | neutral amino acid transporter B (SLC1A5) | + |
| HG4115-HT4385 | Olfactory Receptor Or17-210 | + |
| U25975 | serine kinase (hPAK65) | + |
| U78575 | phosphatidylinositol 4-phosphate 5-kinase alpha PIP5K1A | + |
| U37431 | HOX A1 | + |
| Z26256 | L-type calcium channel/Z26256 | + |
| X54871 | ras-related prot RAB5B | + |
| L10717 | T cell-specific tyrosine kinase | + |
| M20778 | alpha-3 (VI) collagen; Also: X52022 | + |
| D14661 | Splicing regulator WTAP protein/KIAA0105 | + |
| D82344 | NBPhox (PHOX2B) | + |
| D83779 | Hypothetical protein/KIAA0195 | + |
| Z47727 | RNA polymerase II subunit | + |
| D89859 | zinc finger 5 protein (ZNF5) | + |
| Z78289 | (clone 1D2)/Z78289 | + |
| AB002315 | Hypothetical protein/KIAA0317 | + |
| D87969 | CMP-sialic acid transporter (SLC35A1) | + |
| AC002486 | BAC clone RG367O17/7p15-p21/AC002486 | + |
| D38524 | 5-nucleotidase (NT5C2) | + |
| M28879 | granzyme B/CTLA-1 (GZMB) | + |
| Y09392 | WSL-LR, WSL-S1 and WSL-S2 prots; Also: U74611 | + |
| D86967 | alpha mannosidase-like protein/KIAA0212 | + |
| X75755 | PR264; Also: HG3088-HT3261 | + |
| Z49254 | L23-related MRPL23 | − |
| D28364 | annexin II (ANXA2) | + |
| U75272 | gastricsin/progastricsin (PGC); Also: J04443 | + |
| L35253 | p38 mitogen activated prot (MAP) kinase; Also: L35264 | + |
| D50926 | Hypothetical protein/KIAA0136 | + |
| U47635 | D13S824E locus | − |
| U76388 | steroidogenic factor 1 (NR5A1) | + |
| D87076 | Br140/KIAA0239 | + |
| L42324 | (clone GPCR W) G prot-linked receptor/L42324 | + |
| D16626 | Histidine ammonia lyase (HAL) | + |
| D80004 | Hypothetical protein/KIAA0182 | + |
| HG3925-HT4195 | SFTPA2D | + |
| M13699 | ceruloplasmin (CP) | + |
| M90299 | glucokinase (GCK) | + |
| X89267 | DNA uroporphyrinogen decarboxylase (UROD) | + |
| U49835 | YKL-39 precursor; Also: U58514, U58515 | + |
| D28416 | esterase D (ESD) | + |
| U15173 | Nip2 (NIP2) | + |
| X78686 | Chemokine (C-X-C motif) ligand 5 (CXCL5) | + |
| Y14140 | G protein-encoding beta 3 subunit 1 (GNB3) | + |
| X60483 | H4/D histone | + |
| M28219 | low density lipoprot receptor (FH 10) | + |
| D85418 | phosphatidylinositol-glycan-class C (PIGC) | + |
| J04162 | leukocyte IgG receptor (Fc-gamma-R) | + |
| D13988 | rab GDI | + |
| U68233 | farnesol receptor HRR-1 (HRR-1) | + |
| K03218 | src sarcoma viral oncogene homolog (SRC) | + |
| S50223 | HKR-T1 = Kruppel-like zinc finger prot | + |
| X94563 | dbi/acbp/X94563 | + |
| AF001359 | mismatch repair prot (hMLH1)/AF001359 | − |
| U79287 | clone 23867/prostate tumor overexpressed 1 (PTOV1) | − |
| S77583 | HERVK10/HUMMTV reverse transcriptase homolog | + |
| Y00282 | ribophorin II (RPN2) | − |
| S81957 | BMP-5 = bone morphogenic prot-5/S81957 | + |
| AD000092 | RAD23A homolog | + |
| D37781 | protein-tyrosine phosphatase (PTPRJ); Also: U10886 | + |
| L42611 | keratin 6 isoform K6e (KRT6E) | + |
| M80629 | cdc2-like protein kinase 5 (CDC2L5) | + |
| X13255 | dopamine beta-hydroxylase type a (EC 114171) | + |
| Z12962 | homolog to yeast ribosomal prot L41 | + |
| D31883 | actin-binding LIM protein 1 (ABLIM1)/KIAA0059 | + |
| U44799 | U1-snRNP binding prot homolog; Also: U44798 | + |
| X97303 | Ptg-12 prot/X97303 | + |
| U12779 | MAP kinase activated protein kinase 2 (MAPKAPK2) | + |
| M77144 | 3-b-hydroxysteroid DH/5delta-4delta isomerase | + |
| X82240 | T cell leukemia/lymphoma 1 (TCL1A) | + |
| HG3104-HT3280 | Serine Protease Met1 | + |
| U51561 | cosmid N79E2 sequence | + |
| HG4638-HT5050 | Spliceosomal prot Sap 49 | + |
| Z14244 | coxVIIb cytochrome c oxidase subunit VIIb (COX7B) | − |
| M80899 | novel protein desmoyokin (AHNAK) | + |
| X90761 | Keratin, hair, acidic, 2 hHa2 (KRTHA2) | + |
| U91930 | AP-3 complex delta subunit (AP3D1) | − |
| M24461 | pulmonary surfactant-associated prot SP-B (SFTP3) | + |
| U58033 | myotubularin related prot 2 (MTMR2)/U58033 | + |
| HG3521-HT3715 | Ras-Related prot 1b | + |
| D87673 | heat shock transcription factor 4 (HSF4) | + |
| U82535 | fatty acid amide hydrolase (FAAH) | + |
| D88613 | HGCMa/glial cells missing homolog 1 (GCM1) | + |
| X13589 | aromatase (estrogen synthetase) (CYP19A1) | + |
| HG3995-HT4265 | Cpg-Enriched Dna Clone S19 | + |
| U21049 | DD96 | + |
| L13977 | prolylcarboxypeptidase (PRCP) | − |
| HG627-HT5097 | Rhesus (Rh) Blood Group Ce-Antigen, 2, Rhvi | + |
| L76200 | guanylate kinase (GUK1) | − |
| U89896 | casein kinase I gamma 2 (CSNK1G2) | + |
| Y09022 | Not56-like protein (NOT56L) | + |
| X58298 | interleukin-6-receptor (IL6R); Also: M20566 | + |
| X12794 | v-erbA related ear-2/NR2F6 | + |
| D78361 | ornithine decarboxylase antizyme 1 (OAZ1) | + |
| D63998 | golgi alpha-mannosidase II (MAN2A1) | + |
| M14159 | T-cell receptor beta-chain J2.1 | + |
| U79303 | clone 23882 | + |
| X16665 | HOX2H from the Hox2 locus | + |
| X99664 | prot containing SH3 domain SH3GL3 | + |
| X68836 | S-adenosylmethionine synthetase (MAT2A) | + |
| L27943 | cytidine deaminase (CDA) | + |
| D87685 | TFIIS-like PHD finger protein 3/KIAA0244 | + |
| L42563 | (clone ISW34) non-gastric HK-ATPase (ATP1AL1) | + |
| U10886 | Protein tyrosine phosphatase (PTPRJ) | + |
| D42046 | DNA replication helicase-like homolog/KIAA0083 | + |
| D28588 | Sp2 transcription factor/KIAA0048 | + |
| Y10936 | hypothetical prot downstream of DMPK and DMAHP | + |
| U24685 | anti-B cell autoantibody IgM heavy chain V-D-J region | + |

TABLE 8
Intermediate discriminatory genes for MS (on no treatment)
vs. Healthy Donors and ALS

| | | |
|---|---|---|
| Y08265 | DAN26 prot; Also: U94836 | + |
| L14754 | DNA-binding prot (SMBP2) | + |
| U48405 | G prot coupled receptor OGR1 | + |
| M80244 | E16 | + |
| D28114 | myelin-associated oligodendrocytic basic protein | + |
| | (MOBP) | |
| Y09443 | alkyl-dihydroxyacetonephosphate synthase AGPS | + |
| HG2715- | Tyrosine Kinase | + |
| HT2811 | | |
| U70732 | glutamate pyruvate transaminase (GPT) | + |
| U46461 | dishevelled homolog (DVL) | + |
| AF007111 | MDM2-like p53-binding prot (MDMX) | + |
| X06318 | protein kinase C (PKC) type beta I (PRKCB) | + |
| U07807 | metallothionein IV (MTIV) | + |
| J04809 | Cytosolic adenylate kinase AK1 | + |
| Z18954 | S100D calcium binding prot | + |
| U23430 | Cholecystokinin type A receptor (CCKAR); | + |
| | Also: L19315 | |
| Z75330 | nuclear protein stromal antigen SA-1 (STAG1) | + |
| HG315- | Beta-1-Glycoprot 11 Pregnancy-Specific (PSG11) | + |
| HT315 | | |
| U85767 | myeloid progenitor inhibitory factor-1 (MPIF1) | + |
| X82634 | hair keratin acidic 3B (KRTHA3B) | + |
| D50550 | Lethal giant larvae homolog 1 (LLGL1) | + |
| U09550 | oviductal glycoprotein (OVGP1) | + |
| U09303 | T cell leukemia LERK-2 (EPLG2)/Ephrin-B1 | + |
| D28915 | hepatitis C-associated protein p44 (IFI44) | + |
| D38550 | E2f-3 transcription factor/KIAA0075 | + |
| D86979 | hypothetical protein/KIAA0226 | + |
| X05309 | C3b/C4b receptor (CR1) F allotype. | + |
| U88666 | SFRS protein kinase 2 (SRPK2) | + |
| U79294 | clone 23748 phosphatidic acid phosphatase 2B (PPAP2B) | + |
| HG3517- | Alpha-1-Antitrypsin | + |
| HT3711 | | |
| L19593 | IL-8 receptor beta (IL8RB) | + |
| Y07827 | butyrophilin (BTN)/U90552 | + |
| S72487 | Platelet-derived endothelial growth factor 1 (ECGF1) | + |
| M31210 | Endothelial differentiation protein (EDG-1) | + |
| X60655 | EVX1 homeobox | + |
| U41068 | retinoid X receptor beta (RXRbeta)/collagen alpha2(XI) | + |
| X83492 | Fas/APO1 (TNFRSF6) | + |
| HG3991- | Cpg-Enriched Dna, Clone E18 | + |
| HT4261 | | |
| U52154 | G prot-coupled inwardly rectifying K+ channel Kir34 | + |
| HG3627- | Calcium Channel Voltage-Gated B-1 Subunit L Type 2 | + |
| HT3836 | | |
| Y08263 | AAD14 prot | + |
| M16714 | MHC class I divergent lymphocyte antigen; clone RS5 | + |
| Z18956 | taurine transporter (SLC6A6) | + |
| U52827 | Cri-du-chat region clone NIBB11 | + |
| M29971 | 6-O-methylguanine-DNA methyltransferase (MGMT) | − |
| X98534 | Vasodilator-stimulated phosphoprotein (VASP); Z46389 | + |
| U59748 | desert hedgehog (hDHH)/U59748/ | + |
| X58199 | adducin 2 beta (ADD2); Also: S81083_1 | + |
| U27325 | thromboxane A2 receptor (TBXA2R); Also: D38081 | + |
| X81892 | G protein-couped receptor 64 (GPR64) | + |
| L04953 | Amyloid precursor protein binding APBA1 | + |
| X97324 | adipophilin (ADFP) | + |
| Y08319 | kinesin-2 (KIF2) | + |
| HG3638- | Amyloid Beta (A4) Precursor prot; Also: Y00264 | − |
| HT3849 | | |
| M10277 | cytoplasmic beta-actin; Also: HSAC07/X00351 | + |
| U31903 | CREB-RP (CREBL1); Also: U89337_1, X98054 | + |
| D88795 | cadherin | + |
| HG3748- | Basic Transcription Factor 44 Kda Subunit | + |
| HT4018 | | |
| X13967 | leukaemia inhibitory factor (LIF/HILDA) | + |
| X52599 | beta nerve growth factor | + |
| Z25535 | nuclear pore complex prot hnup153 | + |
| M90391 | Interleukin 16 (IL16) | + |
| Z47038 | putative microtubule-associated; prot 1A (MAP1A) | + |
| U43431 | DNA topoisomerase III alpha (TOP3A) | − |
| M63928 | T cell activation antigen (CD27) | + |
| D31797 | CD40 ligand (TNFSF5) | + |
| L13848 | RNA helicase A (DDX9) | + |
| U72512 | B-cell receptor associated prot (hBAP) | + |
| M89957 | B cell receptor complex cell surface glycoprot (IGB) | + |
| M80335 | protein kinase A catalytic subunit (PRKACA) | + |
| L20941 | ferritin heavy chain (FTH1) | − |
| D30655 | eukaryotic initiation factor 4AII | + |
| X80907 | phosphatidyl-inositol-3-kinase p85 (PIK3R2) | + |
| D85181 | fungal sterol-C5-desaturase homolog (SC5DL) | + |
| U58334 | Bcl2 p53 binding prot Bbp/53BP2 (BBP/53BP2) | + |
| U52112 | Renin binding protein (RENBP) | + |
| D25538 | adenylate cyclase 7 (ADCY7)/KIAA0037 | + |
| M98833 | ERGB TRANSCRIPTION FACTORS (FLI-1 homolog) | + |
| Y09943 | NGF-inducible PC3 anti-proliferative protein (BTG2) | + |
| D63160 | DNA lectin P35/Ficolin 2 (FCN2) | + |
| M13955 | mesothelial keratin K7 (type II) | + |
| U03634 | P47 LBC onco | + |
| U05237 | fetal Alz-50-reactive clone 1 (FAC1) | + |
| L41607 | beta-16-N-acetylglucosaminyltransferase (IGnT) | + |
| HG1428- | Globin, Beta; Also: U01317_6 | + |
| HT1428 | | |
| U10868 | aldehyde dehydrogenase ALDH7 | + |
| D42138 | Phosphatidylinositol glycan type B (PIG-B) | + |
| M35999 | platelet glycoprot IIIa/Integrin beta 3 (ITGB3) | + |
| D49490 | disulfide isomerase-related protein (PDIR) | + |
| X06323 | MRL3 ribosomal prot L3 homolog | + |
| Z11502 | annexin A13 (ANXA13) | + |
| X75304 | giantin | − |
| L10955 | carbonic anhydrase IV; Also: M83670 | + |
| J05068 | transcobalamin I | + |
| HG4102- | N-Ethylmaleimide-Sensitive Factor (NSF) | + |
| HT4372 | | |
| Y00486 | adenine phosphoribosyltransferase (aprt) | + |
| U57057 | WD prot IR10 | + |
| X07203 | CD20 receptor (S7) | + |
| X53296 | IRAP; Also: X64532_rna1 X52015 | + |
| U58130 | bumetanide-sensitive Na-K-2Cl cotransporter (NKCC2) | + |
| S71129 | acetylcholinesterase (ACHE) | + |
| U37219 | cyclophilin-like prot CyP-60 | + |
| D31888 | CoREST protein (RCOR)/KIAA0071 | + |
| D25304 | Rac/Cdc42 guanine exchange factor/KIAA0006 | + |
| U89336 | Notch 4 | + |
| U67932 | cAMP phosphodiesterase (Pde7A2)/L12052 | + |
| D63875 | TPR-containing SH2-binding phosphoprotein/KIAA0155 | + |
| U10686 | MAGE-11 antigen (MAGE11) | + |
| S75256 | HNL = neutrophil lipocalin; Also: X99133 | + |
| U78678 | thioredoxin 2 (TXN2) | + |
| L29376 | (clone 38-1) MHC class I fragment | + |
| X77383 | cathepsin-O | + |
| X52730 | phenylethanolamine n-methyltransferase (PNMT) | + |
| HG2167- | prot Kinase Ht31 Camp-Dependent | + |
| HT2237 | | |
| HG3925- | Surfactant prot Sp-A2 Delta/M30838 | + |
| HT4195 | | |
| X74330 | DNA primase (PRIM1) | + |
| HG3994- | Cpg-Enriched Dna Clone S16 | + |
| HT4264 | | |
| M95549 | sodium/glucose cotransporter-like protein (SLC5A2) | + |
| S69790 | Brush-1 = tumor suppressor | + |
| U50553 | helicase like protein 2 (DDX3) | + |
| U63541 | expressed in HC/HCC livers and MolT-4 cells | + |
| K02054 | gastrin-releasing peptide | + |
| D50919 | TRIM14 protein/KIAA0129 | + |
| D50918 | septin 2, 6 (SEPT6)/KIAA0128 | + |
| M91036 | G-gamma globin (HBG2) | + |
| D45371 | GS3109 adipose most abundant gene transcript 1 (APM1) | + |
| D25217 | Membrane protein MLC1/KIAA0027 | + |
| D16217 | Calpastatin (CAST) | + |
| U49973 | tigger 1 transposable element | + |
| L07261 | alpha adducin (ADD1) | + |
| HG3635- | Zinc Finger prot, Kruppel-Like | + |
| HT3845 | | |
| X53586 | integrin alpha 6 | + |
| X13444 | CD8 beta-chain glycoprot (CD8 beta1) | + |
| M29994 | alpha-I spectrin (SPNA1); Also: M61877, M61826 | + |
| HG1102- | Ras-Related C3 Botulinum Toxin Substrate (RAC1) | − |
| HT1102 | | |
| L39059 | TRANSCRIPTION FACTOR SL1 (TAF1C) | − |
| Y10812 | fructose-bisphosphatase | + |
| D90276 | CGM7 nonspecific cross-reacting antigen (NCA) | + |
| S76992 | VAV2 = VAV onco homolog | + |
| S70348 | integrin beta 3 (ITGB3) | + |
| U70323 | ataxin-2 (SCA2) | − |
| U31248 | zinc finger prot (ZNF174) | − |
| HG1783- | Islet Amyloid Polypeptide; Also: X81832 | + |
| HT1803 | | |
| X13293 | v-myb myeloblastosis viral homolog-like 2 (MYBL2) | − |
| Y10517 | CD108 prot/Y10517 | + |
| X65857 | HGMP07E olfactory receptor | + |
| L15309 | zinc finger prot (ZNF141) | + |
| S74728 | antiquitin (ALDH7A1) | − |
| Z70759 | mitochondrial 165 rRNA ()/Z70759 | + |
| U27768 | RGP4 | + |
| X90530 | ragB prot | + |
| D85376 | DNA thyrotropin-releasing hormone receptor (TRHR) | + |
| D26067 | Hypothetical protein/KIAA0033 | + |
| D86976 | minor histocompatibility antigen HA-1/KIAA0223 | + |
| D28423 | pre-splicing factor SRp20 | + |
| HG2602- | Succinate Dehydrogenase Flavoprotein (HSSUCCDH) | + |
| HT2698 | | |
| U06454 | AMP-activated prot kinase (hAMPK) | + |
| M30607 | zinc finger prot Y-linked (ZFY); Also: L10393 | − |
| X02875 | (2-5) oligo A synthetase E (1,8 kb RNA)/M11810_2 | + |
| U43030 | cardiotrophin-1 (CTF1) | + |
| L31584 | G prot-coupled receptor (EBI1) | + |
| D83657 | calcium-binding prot in amniotic fluid 1 CAAF1 | + |
| | (S100A12) | |
| X99586 | SMT3C prot; Also: U67122, U83117, U61397 | + |
| X63097 | Rhesus polypeptide RhXIII | + |
| U82979 | Ig-like transcript-3 (LILRB4) | + |
| D59253 | NCBP interacting prot 1 | + |
| HG4114- | Olfactory Receptor Or17-209 | + |
| HT4384 | | |
| U08815 | splicesomal prot (SAP 61) | − |
| M81637 | Grancalcin (GCA) | + |
| M61855 | cytochrome P4502C9 (CYP2C9), clone 25 | + |
| L38935 | GT212 | + |
| U25265 | MEK5 | + |
| U00928 | clone CE29 4.1 (CAC)n/(GTG)n repeat-containing | + |
| L05628 | multidrug resistance-associated prot (MRP)/X78338 | + |
| D13639 | G1/S-specific cyclin D2/KIAK0002 | + |
| D50525 | TI-227H/D50525 | + |
| S76473 | tyrosine kinase receptor trkB (NTRK2); U12140 | + |
| U33920 | clone lambda 5 semaphorin 3F (SEMA3F) | + |
| M25667 | neuronal growth prot 43 (GAP-43) | + |
| U05040 | FUSE binding protein (FUBP1) | + |
| U70862 | nuclear factor I/B (NFIB) | + |
| X12953 | ab2 , YPT1-related and member of ras family | + |
| AF008937 | syntaxin-16C | + |
| HG4272- | Hepatocyte Growth Factor Receptor | + |
| HT4542 | | |
| X89430 | methyl CpG binding prot 2 | + |
| M27749 | Ig-related 14.1 prot | + |
| D87684 | Hypothetical protein/KIAA0317 | + |
| U33921 | HSU33921 cDNA | + |
| X12530 | B lymphocyte antigen CD20 (B1, Bp35); Also: X07203 | + |
| S50017 | 2,3-cyclic nucleotide 3-phosphodiesterase (CNP) | + |
| U71364 | serine protase inhibitor (P19) | + |
| M27691 | transactivator prot (CREB) | + |
| Z37166 | BAT1 nuclear RNA helicase (DEAD family) | − |
| X54816 | alpha-1-microglobulin-bikunin (AMBP) | + |

TABLE 9
Least discriminatory genes for MS (on no treatment)
vs. Healthy Donors and ALS

| | | |
|---|---|---|
| M16279 | MIC2 | − |
| M14123 | HERV-K10 neutral protease | − |
| U66618 | SWI/SNF complex 60 KDa subunit (BAF60b) | + |
| U37408 | C-terminal binding protein 1 (CTBP1) | − |
| X05196 | aldolase C | + |
| U73304 | CB1 cannabinoid receptor (CNR1). | + |
| L32831 | G prot-coupled receptor (GPR3); Also: U18550 | + |
| D87073 | zinc finger protein znf142/KIAA0236 | + |
| Z50194 | PQ-rich prot | + |
| U38276 | semaphorin III family homolog (SEMA3F) | − |
| S76756 | 4R-MAP2 = microtubule-associated prot 2 4R isoform | + |
| HG1862- | Calmodulin Type I | + |
| HT1897 | | |
| L06175 | P5-1 | + |
| U91932 | AP-3 complex sigma 3A subunit (AP3S1) | − |
| Z84721 | cosmid GG1 from 16p13.3 Contains alpha & zeta globin | + |
| M61733 | erythroid membrane protein 41 (EPB41) | + |
| D86043 | SHPS-1/PTPNS1 tyrosine phosphatase; Also: U06701 | − |
| M65290 | Interleukin 12p40 (IL12p40/IL12B) | + |
| J05016 | prot disulfide isomerase related prot (ERp72) | + |
| J03798 | autoantigen small nuclear ribonucleoprot Sm-D | + |
| HG4128- | Anion Exchanger 3 Cardiac Isom | + |
| HT4398 | | |
| X95808 | prot encoded by DXS6673E/AB002383 | + |
| L40395 | (clone S20iii15) | + |
| K02574 | purine nucleoside phosphorylase (PNP) | + |
| M25809 | endomembrane proton pump subunit | + |
| X06389 | synaptophysin (p38) | + |
| D13370 | APEX nuclease (APEX1) | + |
| V00565 | preproinsulin; Also: M10039 | + |
| U49837 | LIM prot MLP (CSRP3) | + |
| L75847 | zinc finger prot 45 (ZNF45) | + |
| U09087 | thymopoietin (TMPO); Also: U09088 | + |
| U04806 | U03858 and others | + |
| M27093 | alpha-keto acid dehydrogenase transacylase | + |
| HG2479- | Helix-Loop-Helix prot Sef2-1d; Also: M74719 | + |
| HT2575 | | |
| X13930 | CYP2A4 P-450 IIA4 prot | + |
| D42108 | phospholipase C-like 1 (PLCL1) | + |
| M12759 | Ig J chain (IGJ) | + |
| J03764 | Plasminogen activator inhibitor 1 | + |
| L06499 | ribosomal prot L37a (RPL37A) | + |
| HG1595- | Heterogeneous Nuclear Ribonucleoprot I | + |
| HT4788 | | |
| L13329 | iduronate-2-sulfatase (IDS) | + |
| X67683 | keratin 4 (KRT4) | + |
| HG1733- | Moloney Murine Sarcoma Viral Oncogene Homolog | + |
| HT1748 | | |
| M27543 | guanine nucleotide-binding prot (Gi) alpha subunit | − |
| D63506 | unc-18 homolog | + |
| U73477 | acidic nuclear phosphoprot pp32; Also: X75090 | + |
| D87078 | Translational repressor Pumilio/KIAA0235 | + |
| HG3546- | Pre-Splicing Factor Sf2p33 | + |
| HT3744 | | |
| U33052 | lipid-activated, prot kinase PRK2; Also: S75548 | + |
| X57809 | rearranged Ig lambda light chain | + |
| U68536 | zinc finger protein 45 (ZNF45); Also KOX17 | + |
| X53683 | LAG-1 | + |
| L17327 | pre-T/NK cell associated prot (3B3) | + |
| X66867 | MAX | + |
| U07919 | aldehyde dehydrogenase 6 | + |
| S78798 | 1-phosphatidylinositol-4-phosphate 5-kinase isoform C | + |
| M64269 | mast cell chymase; Also: M69137 | − |
| S80905 | PRB2 (PRB2L CON1+) = Con1 | + |
| X52011 | MYF6 encoding a muscle determination factor | + |
| X82693 | Lymphocyte antigen 6 complex, locus D (E48) | + |
| S72503 | HRK1 = inward rectifier potassium channel/U07364 | + |
| S81083 | beta subunit 63 kDa | − |
| M15395 | leukocyte adhesion prot (LFA-1/Mac-1/p150,95) | + |
| X64878 | oxytocin receptor | − |
| D87735 | ribosomal prot L14 | + |
| D43947 | Hypothetical protein/KIAA0100 | + |
| X90846 | mixed lineage kinase 2 (MAP3K10) | + |
| X66142 | rod cGMP phosphodiesterase 6b (PDE6B); Also: S41458 | + |
| HG2992- | Beta-Hexosaminidase Alpha Polypeptide | + |
| HT5186 | | |
| HG64- | NF-Kappa B-Binding protein (KBP1) | − |
| HT64 | | |
| U09953 | ribosomal prot L9 | − |
| D38503 | PMS8 (yeast PMS1 homolog | + |
| D87445 | Hypothetical protein/KIAA0256 | + |
| U82671 | HSP1-A from cosmids from Xq28 | + |
| Z83336 | hH2B/d | + |
| M86406 | skeletal muscle alpha 2 actinin (ACTN20 | − |
| D87845 | platelet-activating factor acetylhydrolase 2 | + |
| X76061 | p130 retinoblastoma-like 2 (RBL2) | + |
| M32886 | sorcin CP-22 (SRI) | − |
| Z49194 | oct-binding factor | + |
| M81830 | somatostatin receptor isom 2 (SSTR2) | + |
| K01911 | neuropeptide Y (NPY) | + |
| U79261 | clone 23959 (MAPK8IP2); Also: U62317 | + |
| D87432 | solute carrier family 7/KIAA0245 | + |
| U55054 | K-Cl cotransporter (hKCC1) | + |
| J00129 | fibrinogen beta-chain | + |
| U49395 | ionotropic ATP receptor P2X5a | + |
| Z80787 | Histone H4 | + |
| D50402 | Natural resistance associated macrophate prot1 | + |
| | (NRAMP1) | |
| M74091 | cyclin C | + |
| D80006 | Hypothetical protein/KIAA0184 | + |
| X17622 | HBK2 potassium channel prot | + |
| Z80780 | H2B/h/Z80780 | + |
| L10338 | sodium channel beta-1 subunit (SCN1B)/U12194/L16242 | + |
| X80026 | B-cam | + |
| HG4321- | Ahnak-Related prot | + |
| HT4591 | | |
| S82297 | beta 2-microglobulin | + |
| HG3033- | Spliceosomal prot Sap 62 | + |
| HT3194 | | |
| U68488 | 5-hydroxytryptamine7 receptor isoform d | + |
| AF002224 | E6-AP ubiquitin prot ligase 3A (UBE3A) | + |
| X84194 | acylphosphatase erythrocyte (CT) isoenzyme | + |
| M76424 | carbonic anhydrase VII (CA VII) | + |
| D30758 | Centaurin beta 1 (CENTB1)/KIAA0050 | + |
| HG907- | Mg44 | + |
| HT907 | | |
| X83490 | Fas/Apo-1/X83490; Also: X83493, X63717 | + |
| M25079 | sickle cell beta-globin | + |
| U82279 | Ig-like transcript 2 (LILRB1) | + |
| U66559 | anaplastic lymphoma kinase receptor | − |
| HG3989- | Cpg-Enriched Dna Clone E14 | + |
| HT4259 | | |
| U20428 | SNC19 sequence | + |
| D50692 | c-myc binding prot | + |
| U18919 | chromosome 17q12-21 clone pOV-2 | − |
| AB002559 | hunc18b2 | + |
| S77576 | ERV9 reverse transcriptase homolog/S77576 | + |
| X12447 | aldolase A (ALDOA) (EC 41213) | − |
| D78367 | K12 keratin | + |
| U22233 | methylthioadenosine phosphorylase (MTAP) | + |
| X97160 | TFE3 transcription factor from TFE3 | + |
| D67029 | SEC14L | + |
| U15197 | histo-blood group ABO prot | + |
| M80478 | platelet glycoprot IX precursor (gpIX) | + |
| AF005775 | caspase-like apoptosis regulatory prot 2 (CLARP) | + |
| Y10514 | CD152 prot (CTLA4); Also: Y10508 | − |
| U51432 | nuclear prot Skip | − |
| M81758 | voltage-dependent sodium channel SKM1 (SCN4A) | + |
| U14973 | ribosomal prot S29 | + |
| U68494 | hbc647 sequence | + |
| U00238 | glutamine PRPP amidotransferase (GPAT) | − |
| U04270 | putative potassium channel subunit (KCNH2) | − |
| M16441 | Lymphotoxin | + |
| D87434 | Hypothetical protein/KIAA0247 | + |
| D13644 | tre oncogene homolog/KIAA0019 | + |
| U20938 | Lymphocyte dihydropyrimidine dehydrogenase (DPYD) | + |
| M94172 | N-type calcium channel alpha-1 subunit (CACNA1B) | + |
| M63959 | alpha-2-macroglobulin receptor-associated prot | − |
| L08069 | heat shock prot E coli DnaJ homolog | + |
| V00594 | metallothionein 2A (MT2A); Also: J00271 | − |
| U09002 | N-methyl-D-aspartate receptor subunit 2A (GRIN2A) | + |
| U01317 | beta-globin thalassemia | + |
| X80692 | ERK3 | − |
| AJ001421 | Rer1 | + |
| U41740 | trans-Golgi p230 (GOLGA4) | − |
| U20760 | extracellular calcium-sensing receptor | + |
| M60830 | Ecotropic viral integration site 2B (EVI2B) | + |
| X60487 | H4/h H4 histone | + |
| L15326 | endoperoxide synthase type II | + |
| Y13618 | DFFRY prot | + |
| D16350 | SA | + |
| X95735 | zyxin 2 | − |
| HG4460- | Ig Heavy Chain Vdjc Regions | + |
| HT4729 | | |
| HG884- | Oncogene E6-Ap, Papillomavirus; Also: U84404 | − |
| HT884 | | |
| M33308 | Vinculin (VCL) | − |
| M27394 | B-lymphocyte cell-surface antigen B1 (CD20) | + |
| U40714 | tyrosyl-tRNA synthetase (YARS) | + |
| U30245 | myelomonocytic specific prot (MNDA)/U30245 | + |
| X80909 | alpha NAC nascent polypeptide-associated complex | − |
| U08096 | peripheral myelin prot-22 (PMP22)/U08096 | − |
| M37435 | macrophage-specific colony-stimulating factor (CSF-1) | + |
| U23435 | Abl interactor 2 (Abi-2); Also: X95632, X95677 | + |
| HG3412- | Blue Cone Photoreceptor Pigment; Also: M13299 | + |
| HT3593 | | |
| D56495 | Reg-related sequence derived peptide-2 (REGL) | + |
| M54995 | connective tissue activation peptide III | + |
| D29956 | ubiquitin specific protease 8/KIAA0055 | + |
| L42572 | p87/89 mitochondrial inner membrane protein (IMMT) | − |
| U83410 | CUL-2 (cul-2) | + |
| K02766 | complement component C9 | − |
| HG4662- | Omega Light Chain Ig Lambda Light Chain Related | + |
| HT5075 | | |
| L49173 | OCP2/L49173 | + |
| L35035 | ribose 5-phosphate isomerase (RPI) | + |
| U15642 | transcription factor E2F-5; Also: U31556 | + |
| AF007551 | Bet1p homolog (hbet1) | + |
| X59711 | CAAT-box DNA binding prot subunit A | + |
| S81243 | CHN = steroid/thyroid orphan receptor homolog/U12767 | + |
| X69636 | sequence (15q11-13) | + |
| U36759 | pre-T cell receptor alpha-type chain precursor (PTCRA) | + |
| U22398 | Cdk-inhibitor p57KIP2 (KIP2) | − |
| U79252 | clone 23679 | + |
| X60188 | ERK1 prot serine/threonine kinase | − |
| D11327 | protein tyrosine phosphatase (PTPN7); Also: M64322 | + |
| Z48923 | BMPR-II | + |
| D21852 | hypothetical protein/KIAA0029 | + |
| X57809 | rearranged Ig lambda light chain; Also: S42404 | + |
| D80007 | RRP5 protein homolog/KIAA0185 | + |
| X71125 | glutamine cyclotransferase (QPCT) | + |
| M31642 | hypoxanthine phosphoribosyltransferase (HPRT) | − |
| U96769 | chondroadherin | + |
| D83174 | collagen binding prot 2 (SERPINH1); Also: X61598 | − |
| M30838 | pulmonary surfactant apoprot (PSAP) | + |
| M60746 | histone H3.1 (H1F3) | + |
| U74382 | telomeric repeat DNA-binding prot (PIN2); | + |
| | Also: U40705 | |
| X16983 | integrin alpha-4 subunit VLA4 (ITGA4) | + |
| U00115 | zinc-finger prot (bcl-6) | − |
| D16181 | PMP2 | + |
| X15875 | cAMP response element binding prot CREBP1 (ATF2) | − |
| HG1103- | Guanine Nucleotide-Binding prot Ral | + |
| HT1103 | | |
| X59812 | CYP 27 vitamin D3 25-hydroxylase | + |
| D49357 | S-adenosylmethionine synthetase | + |
| L24470 | prostanoid FP receptor | + |
| L11701 | phospholipase D; Also: L11702 | + |
| L19183 | MAC30 | − |
| U33822 | tax1-binding prot TXBP181, MAD1-like 1 (MAD1L1) | + |
| Y07829 | RING prot | + |
| U90905 | clone 23574 sequence | + |
| M22995 | ras-related prot (Krev-1) | + |
| U41371 | spliceosome associated prot (SAP 145) | − |
| U79272 | clone 23720 sequence | + |
| M13929 | c-myc-P64/HG3523-HT4900/HG3523-HT4899/L00058 | + |
| Z19554 | vimentin (VIM); Also: M18895_2 | − |
| M30269 | nidogen | + |
| AB000449 | VRK1 | + |
| X07618 | cytochrome P450 db1 variant a/X07619/X16866/M33189 | + |
| D79986 | bcl-2-associated transcription factor/KIAA0164 | + |
| U76010 | putative zinc transporter ZnT-3 (ZnT-3) | + |
| Z38026 | FALL-39 peptide antibiotic | − |
| U79255 | X11 prot | − |
| X79865 | Mrp17/Mitochondrial ribosomal L12 (MRPL12) | − |
| M81750 | myeloid cell nuclear differentiation antigen | + |
| U73377 | p66shc (SHC) | − |
| U43965 | ankyrin G119 (ANK3) | + |
| U03688 | dioxin-inducible cytochrome P450 (CYP1B1) | + |
| U47050 | putative calcium influx channel (htrp3) | − |
| M19645 | 78 kdalton glucose-regulated prot (GRP78) | − |
| X95404 | non-muscle type cofilin | − |
| D50640 | phosphodiesterase 3B (PDE3B) | + |
| M62831 | TRANSCRIPTION FACTORS ETR101 | + |
| U06863 | follistatin-related protein precursor (FSTL1) | + |
| L11672 | Kruppel related zinc finger prot (HTF10) | + |
| M80397 | DNA polymerase delta catalytic subunit; Also: M81735 | − |
| D10656 | Sarcoma virus homolog (CRK) | + |
| D86959 | hSLK serine threonine kinase/KIAA0204 | + |
| J02982 | glycophorin B | + |
| X81333 | PPH beta subunit prot | + |
| X76105 | Death-associated protein (DAP1) | − |
| M92642 | alpha-1 type XVI collagen (COL16A1), | + |
| U38175 | HuR RNA binding prot (HuR) | − |
| HG3636- | Myosin, Heavy Polypeptide 9, Non-Muscle | + |
| HT3846 | | |
| L37378 | guanylyl cyclase (RetGC-2) | − |
| X82200 | Staf50 | + |
| Y08409 | spot14 | + |
| D38583 | calgizzarin | + |
| D78514 | ubiquitin-conjugating enzyme | + |
| M31627 | X box binding prot-1 (XBP-1) | + |
| U81556 | hypothetical prot A4 | + |
| U07550 | Chaperonin 10 (HSPE1) | − |
| X52943 | ATF-a TRANSCRIPTION FACTORS | − |
| X95654 | SCP1 prot | + |
| L16842 | ubiquinol cytochrome-c reductase core l prot (UQCRC1) | − |
| M26004 | CR2/CD21/C3d/Epstein-Barr virus receptor/J03565 | + |
| D28476 | thyroid hormone receptor interactor 12/KIAA0045 | + |
| X80590 | PHKG1 | + |
| X59842 | PBX2; Also: U89336_2, D28769_1, X80700_rna1 | − |
| Y07847 | RRP22 prot | + |
| U15306 | cysteine-rich sequence-specific DNA-binding prot NFX1 | + |
| L39874 | deoxycytidylate deaminase | + |
| D16154 | cytochrome P-450c11/D16154 | + |
| X98253 | ZNF183/X98253 | + |
| M59941 | GM-CSF receptor beta chain (CSF2RB) | + |
| L10615 | beta casein (CSN2); Also: X17070 | + |
| X64728 | CHML | + |
| S41458 | rod cGMP phosphodiesterase 6B (PDE6B) | + |
| X87176 | 17-beta-hydroxysteroid dehydrogenase | + |
| HG3884- | Homeotic protein HPX42 | − |
| HT4154 | | |
| U15174 | Nip3 (NIP3) | + |
| D64015 | T-cluster binding protein/D64015 | + |
| U80040 | aconitase nuclear encoded mitochondrial prot | − |
| U19878 | transmembrane prot | + |
| D83018 | nel-related prot 2 | + |
| M22538 | mitochondrial NADH-ubiquinone reductase | − |
| | 24 Kd subunit | |
| U37055 | hepatocyte growth factor-like prot/L11924/M74178 | − |
| M75715 | TB3-1; Also: X81625 | − |
| U02566 | receptor tyrosine kinase TIF; Also: U18934 | − |
| X74142 | HBF-1 TRANSCRIPTION FACTORS | + |
| M16750 | pim-1 oncogene; Also: M27903, M24779, M54915 | + |
| U73799 | dynactin/U73799 | + |
| V01516 | cytoskeletal keratin (type II) from foreskin | + |
| J02960 | unknown prot/M15169 | + |
| U34380 | prot tyrosine kinase TEC and TXK (txk)/D29767 | + |
| S78432 | transmembrane 4 prot/S78432 | − |
| U70439 | silver-stainable prot SSP29; Also: Y07570 | − |
| U90916 | clone 23815 sequence, lFN-inducible | + |
| M99438 | transducin-like enhancer prot (TLE3) | + |
| X98248 | Sortilin (SORT1) | + |
| L11931 | cytosolic serine hydroxymethyltransferase (SHMT) | + |
| U68485 | Box-dependent MYC-interacting prot-1 (BIN1) | + |
| X14975 | CD1 R2 MHC-related antigen | + |
| S75578 | 4-aminobutyrate aminotransferase (ABAT) | − |
| D28235 | PTGS2 prostaglandin endoperoxide synthase-2/U04636 | + |
| X99656 | prot containing SH3 domain SH3GL1 | + |
| U08854 | UDP glucuronosyltransferase UGT2B15/U06641 | + |
| D14889 | small GTP-binding protein S10 (RAB33A) | + |
| M31899 | DNA repair helicase (ERCC3) | − |
| M75099 | Rapamycin and FK506-binding protein FKBP13 | − |
| Z68193 | Opsin 1 (OPN1LW) | + |
| U01212 | olfactory marker prot (OMP) | + |
| X74331 | DNA primase (subunit p58) | + |
| M91029 | AMP deaminase (AMPD2) | + |
| U63295 | seven in absentia homolog | − |
| U58970 | outer mitochondrial membrane translocase (TOMM34) | − |
| L08010 | reg homolog | + |
| M67468 | Fragile X mental retardation 1 FMR-1/X69962 | + |
| M13829 | putative raf related prot (pks/a-raf); Also: U01337 | + |
| U16811 | Bak; Also: U23765 | − |
| U34877 | biliverdin-IXalpha reductase | − |
| D16593 | BDR-2 hippocalcin | + |
| L19871 | activating TRANSCRIPTION FACTORS 3 (ATF3) | − |
| D86964 | dedicator of cytokinesis 1 homolog protein/KIAA0209 | + |
| U80017 | Survival motor neuron protein (SMN) | − |
| M81780 | Sphingomyelin phosphodiesterase 1 (SMPD1) | + |
| D42044 | hypothetical protein/KIAA0090 | + |
| Y07701 | aminopeptidase | + |
| D29954 | hypothetical protein/KIAA0056 | + |
| X61373 | alternatively spliced tau/X61373 | + |
| K01884 | Blym-1 transforming | + |
| L37792 | syntaxin 1A | + |
| X65488 | Heterogeneous nuclear ribonucleoprotein U (HNRPU) | + |
| J05243 | nonerythroid alpha-spectrin (SPTAN1) | − |
| D63485 | Inhibitor of NFkB kinase epsilon subU. | + |
| | IkBKE/KIAA0151 | |
| M60750 | histone H2B.1 (H2B)/M60750 | + |
| D13897 | peptide YY precursor | + |
| X04470 | antileukoprotease (ALP) from cervix uterus/X04503 | + |
| U65581 | ribosomal prot L3-like | + |
| U35113 | metastasis-associated mta1 | − |
| X62822 | beta-galactoside alpha-26-sialyltransferase | + |
| D50477 | membrane-type matrix metalloproteinase 3/D83646 | + |
| U59736 | TRANSCRIPTION FACTORS (NFATcb) | + |
| X07173 | second prot of inter-alpha-trypsin inhibitor complex | + |
| X52638 | 6-phosphofructo-2-kinase/fructose-26-bisphosphatase | + |
| Z46632 | HSPDE4C1 3,5-cyclic AMP phosphodiesterase (PDE4C) | + |
| D38024 | facioscapulohumeral muscular dystrophy (FSHD) | + |
| D87435 | golgi-specific brefeldin A resistance factor/KIAA0248 | + |
| L20859 | leukemia virus receptor 1 GLVR1 (SLC20A1) | + |
| AF006087 | Arp2/3 prot complex subunit p20-Arc (ARC20) | + |
| U83239 | CC chemokine STCP-1; Also: U83171 | + |
| D13435 | phosphatidyl-inositol-glycan class F | + |
| M37815 | CD28 | + |
| X03342 | ribosomal prot L32 | − |
| D88797 | cadherin | + |
| U85430 | TRANSCRIPTION FACTORS NFATx4 | + |
| U96131 | HPV16 E1 prot binding prot/U96131 | + |
| X62078 | GM2 activator protein (GM2A) | − |
| U62389 | cytosolic NADP-dependent isocitrate dehydrogenase | + |
| U02680 | protein tyrosine kinase 9 (PTK9) | − |
| U89922 | lymphotoxin beta isoform variant/L11016_rna1, L11015 | + |
| Y08999 | Sop2p-like prot | − |
| D83032 | nuclear prot NP220 | + |
| U66077 | DAZ, 3 UTR | + |
| D86960 | Hypothetical protein/KIAA0205 | + |
| U86136 | telomerase-associated prot TP-1 | + |
| U51010 | nicotinamide N-methyltransferase/U08021 | − |
| X65644 | MBP-2 MHC binding prot 2 | + |
| M18391 | tyrosine kinase receptor (eph); Also: Z27409 | + |
| L20298 | TRANSCRIPTION FACTORS (CBFB) | + |
| U62801 | protease M | + |
| M93405 | methylmalonate semialdehyde dehydrogenase | + |
| | (ALDH6A1) | |
| D26561 | ORF E7 from papillomavirus 5b genome | + |
| U37352 | prot phosphatase 2AB alpha 1 | + |
| D00760 | proteasome subunit HC3 | + |
| U88726 | symplekin/U88726 | + |
| Y09615 | mitochondrial transcription termination factor (MTERF) | + |
| M65214 | (HeLa) helix-loop-helix prot HE47 (E2A); Also: M31523 | + |
| X15393 | motilin | + |
| X59373 | HOX4D a homeobox prot | + |
| M29696 | interleukin-7 receptor (IL-7) | + |
| X07834 | Manganese superoxide dismutase SOD2 | + |
| U43203 | thyroid transcription factor 1 (TTF-1); Also: U33749 | + |
| U01923 | BTK region clone ftp-3 | + |
| X97444 | transmembrane prot Tmp21-Ilex./X97444 | + |
| D49489 | disulfide isomerase-related prot P5 | + |
| M54915 | h-pim-1 prot (h-pim-1); Also: M27903, M24779 | + |
| X05345 | histidyl-tRNA synthetase (HRS) | − |
| U94585 | requiem homolog (hsReq) | + |
| D88799 | cadherin | + |
| S81916 | phosphoglycerate kinase | + |
| J04088 | DNA topoisomerase II (top2) | + |
| D83597 | RP105 | + |
| X97230 | NK receptor, clone library 4M1#6 | + |
| HG1155- | Colony-Stimulating Factor 1 Macrophage (CSF1) | + |
| HT4822 | | |
| U31342 | nucleobindin | + |
| M13207 | granulocyte-macrophage colony-stimulating factor CSF1 | − |
| U24704 | antisecretory factor-1 (PSMD4) | − |
| U14391 | myosin-IC | − |
| L10405 | DNA binding prot surfactant prot B/L10405 | − |
| U21943 | organic anion transporting polypeptide (OATP) | + |
| U58087 | Hs-cul-1 | + |
| M21188 | insulin-degrading enzyme (IDE) | − |
| D42040 | RING3 protein/KIAA9001; Also: X62083, M80613 | + |
| HG1602- | Utrophin | + |
| HT1602 | | |
| X61072 | T cell receptor, clone IGRA17 | + |
| M60298 | erythrocyte membrane prot band 42 (EPB42) | + |
| U79280 | clone 23575 | + |
| HG3141- | Nadh-Ubiquinone Oxidoreductase, 39 Kda Subunit | + |
| HT3317 | | |
| U52077 | mariner1 transposase/U52077; Also: U80776 | − |
| L22454 | nuclear respiratory factor-1 (NRF-1) | + |
| Y00503 | keratin 19 | + |
| HG2139- | Beta-1-Glycoprot 1, Pregnancy-Specific/M25384 | + |
| HT2208 | | |
| M58026 | NB-1 | − |
| HG2320- | Integrin Beta 3 Subunit | + |
| HT2416 | | |
| S69265 | neuron-specific RNA recognition motifs; Also: L26405 | + |
| M93284 | pancreatic lipase related prot 2 (PLRP2) | + |
| M75110 | HK-ATPase beta subunit | + |
| M19283 | cytoskeletal gamma-actin | − |
| L46720 | autotaxin-t (atx-t); Also: L35594 | − |
| H46990 | CYT P450 IIE1 homolog | + |
| X76059 | YRRM1 | − |
| AC002086 | PAC clone DJ525N14/Xq23 | + |
| M17885 | acidic ribosomal phosphoprot P0 | − |
| U64863 | hPD-1 (hPD-1) | − |
| M80333 | m5 muscarinic acetylcholine receptor | + |
| U40705 | telomeric repeat binding factor (TRF1) | − |
| Y09980 | HOXD3 | − |
| D49818 | fructose 6phosphate 2kinase/fructose2 6bisphosphatase | − |
| HG4263- | Nkr-P1a prot | + |
| HT4533 | | |
| M29277 | isolate JuSo MUC18 glycoprot (3′ variant) | − |
| Y10615 | CYRN2/Y10615 | + |
| D83785 | mam1 mastermind homolog/KIAA0200 | + |
| J02611 | apolipoprot D | + |
| D87119 | cancellous bone osteoblast | + |
| U07857 | 18 kDa Alu RNA binding prot | − |
| U94332 | osteoprotegerin (OPG) | − |
| D42053 | Site-1 protease transcription factor/KIAA0091 | + |
| J05272 | IMP dehydrogenase type 1 | + |
| M29610 | glycophorin E | + |
| U71300 | snRNA activating prot complex 50 kD subunit (SNAP50) | + |
| M98776 | keratin 1 | + |
| L22650 | early lymphoid activation prot (EPAG) | + |
| M23533 | alpha 2 adrenergic receptor | + |
| L14922 | DNA-binding prot (PO-GA) | + |
| X95073 | translin associated prot X | + |
| X66087 | a-myb | + |
| U79267 | clone 23840 | − |
| X85786 | DNA binding regulatory factor | + |
| M57423 | phosphoribosylpyrophosphate synthetase subunit III | + |
| HG3214- | Metallopanstimulin 1 | + |
| HT3391 | | |
| Z29064 | AF-1p | + |
| D86425 | osteoblast osteonidogen | − |
| X95152 | brca2 | + |
| J04617 | elongation factor EF-1-alpha; Also: M27364 | + |
| X95826 | ART4/X95826 | + |
| X52221 | ERCC2 | + |
| U40215 | synapsin IIb | + |
| HG4167- | Nuclear Factor 1 A Type | + |
| HT4437 | | |
| M29540 | carcinoembryonic antigen (CEA) | + |
| X77166 | kunitz-type protease inhibitor HKIB9 | + |
| D49728 | NAK1 DNA binding prot, | + |
| X98258 | M-phase phosphoprot mpp9 | + |
| HG3123- | Homeotic prot Gbx2 | + |
| HT3299 | | |
| M91463 | glucose transporter (GLUT4) | + |
| HG1140- | Collagen, Type Vi, Alpha 2; Also: M34570 | − |
| HT4817 | | |
| X54150 | Fc receptor | − |
| X90857 | (−14) containing globin regulatory element (CGTHBA) | − |
| U67611 | Mouse transaldolase/U67611 | + |
| M34175 | beta adaptin | + |
| J02883 | Colipase (CLPS) | + |
| U34976 | gamma-sarcoglycan (SGCG) | − |
| Z80788 | H4/I | + |
| U89335 | Notch 4 | + |
| X06268 | pro-alpha 1 (II) collagen | + |
| X94910 | ERp31 prot | + |
| U80811 | Lysophosphatidic acid receptor homolog (EDG2) | − |
| U52153 | inwardly rectifying potassium channel Kir32 | − |
| U14577 | microtubule-associated prot 1A (MAP1A)/U38291_rnal | + |
| M34376 | beta-microseminoprot (MSP); Also: X57928 | + |
| U68723 | checkpoint suppressor 1 | − |
| L11708 | 17 beta hydroxysteroid dehydrogenase type 2 | + |
| U58034 | myotubularin related prot 3 (MTMR3)/U58034 | − |
| Z22551 | kinectin | − |
| D50915 | hypothetical protein/KIAA0125 | + |
| U57352 | sodium channel 1 (hBNaC1) | + |
| M62324 | modulator recognition factor I (MRF-1) | − |

TABLE 10
Highly discriminatory genes for MS (regardless of treatment with Avonex or
not) vs. Healthy Donors and ALS

| Probe sets | Gene Descriptions | Up (+) or down (−) |
|---|---|---|
| Z16411 | phospholipase c; Also: U26425, Z37544 (PLCB3) | − |
| U78095 | Placental bikunin (AMBP) | + |
| L34075 | FKBP-rapamycin associated prot (FRAP) | + |
| D79993 | Hypothetical/KIAA0171/Enthoprotin (ENTH) | + |
| L42324 | (clone GPCR W) G prot-linked receptor/L42324 | + |
| D29675 | iNOS | + |
| U79528 | SR31747 binding prot 1 (SRBP1); Also: U75283 | + |
| U39318 | E2 ubiquitin conjugating enzyme UbcH5C (UBCH5C) | + |
| M19650 | 2,3-cyclic nucleotide 3-phosphodiesterase (CNP) | + |
| Y09392 | WSL-LR, WSL-S1 and WSL-S2 prots; Also: U74611 | + |
| HG3991-HT4261 | Cpg-Enriched Dna, Clone E18 | + |
| HG2415-HT2511 | Transcription Factor E2f-2 | + |
| M14159 | T-cell receptor beta-chain J2.1 | − |
| D30037 | phosphatidylinositol transfer protein (PITPN) | + |
| U63717 | osteoclast stimulating factor (OSTF1) | + |
| HG3730-HT4000 | Tyrosine Kinase Syk; Also: L28824 | + |
| U89896 | casein kinase l gamma 2 (CSNK1G2) | + |
| D16626 | Histidine ammonia lyase (HAL) | + |
| D86967 | alpha mannosidase-like protein/KIAA0212 | + |
| Z26256 | L-type calcium channel/Z26256 | + |
| L34357 | GATA-4 | + |
| Y00282 | ribophorin II (RPN2) | − |
| D83784 | C2 H2-type zinc finger protein/KIAA0198 | + |
| U66059 | TCRBV1S1A1N1 from germline T-cell receptor beta chain | + |
| HG3175-HT3352 | Carcinoembryonic Antigen | + |
| HG3521-HT3715 | Ras-Related prot 1b | + |
| M90299 | glucokinase (GCK) | + |
| M25269 | tyrosine kinase (ELK1) onco | + |
| U14187 | receptor tyrosine kinase ligand LERK-3/Ephrin-A3 | + |
| D26069 | Centaurin beta 2 (CENTB2)/KIAA0041 | + |
| X72879 | 14A2AK DNA sequence | + |
| D13988 | rab GDl | + |
| U34301 | nonmuscle myosin heavy chain IIBU34301 | + |
| Z49254 | L23-related MRPL23 | − |
| Z46632 | HSPDE4C1 3,5-cyclic AMP phosphodiesterase (PDE4C) | + |
| M80335 | protein kinase A catalytic subunit (PRKACA) | + |
| X65977 | corticostatin HP-4 precursor (defensin/DEFA4) | + |
| U53347 | neutral amino acid transporter B (SLC1A5) | + |
| X83492 | Fas/APO1 (TNFRSF6) | + |
| HG4108-HT4378 | Olfactory Receptor Or17-24 | + |
| X58298 | interleukin-6-receptor (IL6R); Also: M20566 | + |
| X07619 | cytochrome P450 db1 variant b; Also: X16866 | + |
| D87450 | parallel sister chromatids drosophila protein-like/KIAA0261 | + |
| U44799 | U1-snRNP binding prot homolog; Also: U44798 | + |
| Z15114 | protein kinase C gamma (PRKCG) | + |
| U22028 | cytochrome P450 (CYP2A13) | + |
| HG1862-HT1897 | Calmodulin Type I | + |
| U03398 | receptor 4-1BB ligand (TNFSF9) | + |
| D38491 | Hypothetical protein/KIAA0117 | + |
| U79287 | clone 23867/prostate tumor overexpressed 1 (PTOV1) | − |
| D78361 | ornithine decarboxylase antizyme 1 (OAZ1) | + |
| D87969 | CMP-sialic acid transporter (SLC35A1) | + |
| L05512 | histatin 1 (HTN1) | + |
| HG2825-HT2949 | Ret Transforming | + |
| U59752 | Sec7p-like prot | + |
| U78575 | phosphatidylinositol 4-phosphate 5-kinase alpha PIP5K1A | + |
| D26579 | transmembrane prot ADAM8 | + |
| D25304 | Rac/Cdc42 guanine exchange factor/KIAA0006 | + |
| M35416 | GTP-binding prot (RALB) | + |
| U18271 | thymopoietin (TMPO); Also: U09087, U09088 | + |
| S74728 | antiquitin (ALDH7A1) | − |
| L36922 | Met-ase 1/Granzyme M (GZMM) | + |
| L77561 | DiGeorge syndrome critical region 11 DGS-D (DGCR11) | + |
| X94563 | dbi/acbp/X94563 | + |
| D45132 | kidney zinc-finger DNA-binding protein PRDM2 | + |
| M34181 | testis-specific cAMP-dependent prot kinase (PRKACB) | + |
| X57809 | rearranged Ig lambda light chain | + |
| U70732 | glutamate pyruvate transaminase (GPT) | + |
| U47635 | D13S824E locus | − |
| M29971 | 6-O-methylguanine-DNA methyltransferase (MGMT) | − |
| L27943 | cytidine deaminase (CDA) | + |
| U31248 | zinc finger prot (ZNF174) | − |
| HG315-HT315 | Beta-1-Glycoprot 11 Pregnancy-Specific (PSG11) | + |
| HG4115-HT4385 | Olfactory Receptor Or17-210 | + |
| HG2715-HT2811 | Tyrosine kinase | + |
| Z25884 | CIC-1 muscle chloride channel protein (CLCN1) | + |
| L76200 | guanylate kinase (GUK1) | − |
| K03218 | src sarcoma viral oncogene homolog (SRC) | + |
| HG2167-HT2237 | prot Kinase Ht31 Camp-Dependent | + |
| U46461 | dishevelled homolog (DVL) | + |
| Z12962 | homolog to yeast ribosomal prot L41 | + |
| U76388 | steroidogenic factor 1 (NR5A1) | + |
| M13994 | bcl-2-alpha; Also: M14745 | + |
| U49835 | YKL-39 precursor; Also: U58514, U58515 | + |
| Y08265 | DAN26 prot; Also: U94836 | + |
| D30036 | phosphatidylinositol transfer protein (PITPN) | + |
| M86406 | skeletal muscle alpha 2 actinin (ACTN20 | − |
| J04162 | leukocyte IgG receptor (Fc-gamma-R) | + |
| U50743 | NaK-ATPase gamma subunit | + |
| AC002486 | BAC clone RG367O17/7p15-p21/AC002486 | + |
| D64158 | cell differentiation-associated ATP binding prot | + |
| D86976 | KIAA0223 | + |
| U91930 | AP-3 complex delta subunit (AP3D1) | − |
| M20778 | alpha-3 (VI) collagen; Also: X52022 | + |
| M27749 | Ig-related 14.1 prot | + |
| D28423 | pre-splicing factor SRp20 | + |
| D86043 | SHPS-1/PTPNS1 tyrosine phosphatase; Also: U06701 | − |
| M10277 | cytoplasmic beta-actin; Also: HSAC07/X00351 | + |
| HG3995-HT4265 | Cpg-Enriched Dna Clone S19 | + |
| X68836 | S-adenosylmethionine synthetase (MAT2A) | + |
| U20647 | zinc finger prot (ZNF151) | + |
| Z14244 | coxVIIb cytochrome c oxidase subunit VIIb (COX7B) | − |
| D28416 | esterase D (ESD) | + |
| D31797 | CD40 ligand (TNFSF5) | + |
| U79303 | clone 23882 | + |
| D29833 | salivary proline rich peptide P-B | + |
| S82297 | beta 2-microglobulin | + |
| AD000092 | RAD23A homolog | + |
| D86979 | hypothetical protein/KIAA0226 | + |
| M13577 | myelin basic prot (MBP) | + |
| AF008937 | syntaxin-16C | + |
| S76992 | VAV2 = VAV onco homolog | + |
| X12794 | v-erbA related ear-2/NR2F6 | + |
| X05309 | C3b/C4b receptor (CR1) F allotype. | + |
| D28588 | Sp2 transcription factor/KIAA0048 | + |
| HG3627-HT3836 | Calcium Channel Voltage-Gated Beta 1 Subunit L Type 2 | + |
| X99586 | SMT3C prot; Also: U67122, U83117, U61397 | + |
| M19645 | 78 kdalton glucose-regulated prot (GRP78) | − |
| Y09022 | Not56-like protein (NOT56L) | + |
| X66867 | MAX | + |
| X75755 | PR264; Also: HG3088-HT3261 Same Uni Cluster as M90104 | + |
| X52599 | beta nerve growth factor | + |
| U68536 | zinc finger protein 45 (ZNF45); Also KOX17 | + |
| U09550 | oviductal glycoprotein (OVGP1) | + |
| K01884 | Blym-1 transforming | + |
| D50640 | phosphodiesterase 3B (PDE3B) | + |
| M29696 | interleukin-7 receptor (IL-7) | + |
| X02875 | (2-5) oligo A synthetase E (1,8 kb RNA); Also: M11810_2 | + |
| U75272 | gastricsin/progastricsin (PGC); Also: J04443 | + |
| HG3991-HT4261 | Cpg-Enriched Dna, Clone E18 | + |
| X75304 | giantin | − |
| M63959 | alpha-2-macroglobulin receptor-associated prot | − |
| Z70759 | mitochondrial 16S rRNA ()/Z70759 | + |
| Y08409 | spot14 | + |
| D49487 | obese/Leptin (LEP); Also: U43653 | + |
| X13444 | CD8 beta-chain glycoprot (CD8 beta1) | + |
| S82362 | hRAR- beta 2 = retinoic-acid-receptor beta/S82362/M62303 | + |
| X52943 | ATF-a TRANSCRIPTION FACTORS | − |
| D31883 | actin-binding LIM protein 1 (ABLIM1)/KIAA0059 | + |
| D87735 | ribosomal prot L14 | + |
| X15875 | cAMP response element binding prot CREBP1 (ATF2) | − |
| D86964 | dedicator of cytokinesis 1 homolog protein/KIAA0209 | + |
| U41371 | spliceosome associated prot (SAP 145) | − |
| Y09443 | alkyl-dihydroxyacetonephosphate synthase AGPS | + |
| L39059 | TRANSCRIPTION FACTOR SL1 (TAF1C) | − |
| U03634 | P47 LBC onco | + |
| L14754 | DNA-binding prot (SMBP2) | + |
| D37781 | protein-tyrosine phosphatase (PTPRJ); Also: U10886 | + |
| M62831 | TRANSCRIPTION FACTORS ETR101 | + |
| U48405 | G prot coupled receptor OGR1 | + |
| M16279 | MIC2 | − |
| M13699 | ceruloplasmin (CP) | + |
| U73477 | acidic nuclear phosphoprot pp32; Also: X75090 | + |
| U15197 | histo-blood group ABO prot | + |
| Z18954 | S100D calcium binding prot | + |
| X99664 | prot containing SH3 domain SH3GL3 | + |
| U73377 | p66shc (SHC) | − |
| D87434 | Hypothetical protein/KIAA0247 | + |
| D11327 | protein tyrosine phosphatase (PTPN7); Also: M64322 | + |
| D87445 | Hypothetical protein/KIAA0256 | + |
| X82200 | Staf50 | + |
| Y09943 | NGF-inducible PC3 anti-proliferative protein (BTG2) | + |
| L08069 | heat shock prot E coli DnaJ homolog | + |
| L13848 | RNA helicase A (DDX9) | + |

TABLE 11
Intermediate discriminatory genes for MS (regardless of treatment with Avonex
or not) vs. Healthy Donors and ALS

| | | |
|---|---|---|
| M81750 | myeloid cell nuclear differentiation antigen | + |
| D83018 | nel-related prot 2 | + |
| M28219 | low density lipoprot receptor (FH 10) | + |
| M81637 | Grancalcin (GCA) | + |
| D25538 | adenylate cyclase 7 (ADCY7)/KIAA0037 | + |
| D87673 | heat shock transcription factor 4 (HSF4) | + |
| X98534 | Vasodilator-stimulated phosphoprotein (VASP); Z46389 | + |
| L31584 | G prot-coupled receptor (EBI1) | + |
| L17327 | pre-T/NK cell associated prot (3B3) | + |
| X90761 | Keratin, hair, acidic, 2 hHa2 (KRTHA2) | + |
| L29376 | (clone 38-1) MHC class I fragment | + |
| M98833 | ERGB TRANSCRIPTION FACTORS (FLI-1 homolog) | + |
| U50553 | helicase like protein 2 (DDX3) | + |
| U20428 | SNC19 sequence | + |
| M75099 | Rapamycin and FK506-binding protein FKBP13 | − |
| L10717 | T cell-specific tyrosine kinase | + |
| X80692 | ERK3 | − |
| L35253 | P38 mitogen activated prot (MAP) kinase; Also: L35264 | + |
| L19593 | IL-8 receptor beta (IL8RB) | + |
| Y00486 | adenine phosphoribosyltransferase (aprt) | + |
| D38524 | 5 -nucleotidase (NT5C2) | + |
| M90391 | Interleukin 16 (IL16) | + |
| M32886 | sorcin CP-22 (SRI) | − |
| U09303 | T cell leukemia LERK-2 (EPLG2)/Ephrin-B1 | + |
| L15309 | zinc finger prot (ZNF141) | + |
| L06499 | ribosomal prot L37a (RPL37A) | + |
| D13639 | G1/S-specific cyclin D2/KIAK0002 | + |
| L42563 | (clone ISW34) non-gastric HK-ATPase (ATP1AL1) | + |
| U52112 | Renin binding protein (RENBP) | + |
| L20859 | leukemia virus receptor 1 GLVR1 (SLC20A1) | + |
| L38935 | GT212 | + |
| M13955 | mesothelial keratin K7 (type II) | + |
| Z47727 | RNA polymerase II subunit | + |
| U10886 | Protein tyrosine phosphatase (PTPRJ) | + |
| D30655 | eukaryotic initiation factor 4AII | + |
| X60188 | ERK1 prot serine/threonine kinase | − |
| D80006 | Hypothetical protein/KIAA0184 | + |
| X89267 | DNA uroporphyrinogen decarboxylase (UROD) | + |
| Z38026 | FALL-39 peptide antibiotic | − |
| U78678 | thioredoxin 2 (TXN2) | + |
| M15395 | leukocyte adhesion prot (LFA-1/Mac-1/p150,95 family) | + |
| M60626 | N-formylpeptide receptor 1 (FPR1) | + |
| S81083 | beta subunit 63 kDa | − |
| S77583 | HERVK10/HUMMTV reverse transcriptase homolog/S77583 | + |
| U25975 | serine kinase (hPAK65) | + |
| HG3994-HT4264 | Cpg-Enriched Dna Clone S16 | + |
| HG627-HT5097 | Rhesus (Rh) Blood Group Ce-Antigen, 2, Rhvi; Also: X63097 | + |
| U52827 | Cri-du-chat region clone NIBB11 | + |
| M60830 | Ecotropic viral integration site 2B (EVI2B) | + |
| U61500 | GT334 prot (GT334) | + |
| U43431 | DNA topoisomerase III alpha (TOP3A) | − |
| M31210 | Endothelial differentiation protein (EDG-1) | + |
| U51432 | nuclear prot Skip | − |
| U27325 | thromboxane A2 receptor (TBXA2R); Also: D38081 | + |
| X97303 | Ptg-12 prot/X97303 | + |
| D28364 | annexin II (ANXA2) | + |
| D43947 | Hypothetical protein/KIAA0100 | + |
| D89859 | zinc finger 5 protein (ZNF5) | + |
| HG3517-HT3711 | Alpha-1-Antitrypsin | + |
| U37055 | hepatocyte growth factor-like prot; Also: L11924, M74178 | − |
| L10955 | carbonic anhydrase IV; Also: M83670 | + |
| D42046 | DNA replication helicase-like homolog/KIAA0083 | + |
| S72503 | HRK1 = inward rectifier potassium channel; Also: U07364 | + |
| D83920 | uterus ficolin-1 | + |
| M64269 | mast cell chymase; Also: M69137 | − |
| U66618 | SWI/SNF complex 60 KDa subunit (BAF60b) | + |
| M24461 | pulmonary surfactant-associated prot SP-B (SFTP3) | + |
| D80004 | Hypothetical protein/KIAA0182 | + |
| U41068 | retinoid X receptor beta (RXRbeta)/collagen alpha2(XI) | + |
| D83657 | calcium-binding prot in amniotic fluid 1 CAAF1 (S100A12) | + |
| X53586 | integrin alpha 6 | + |
| K02054 | gastrin-releasing peptide | + |
| X94910 | ERp31 prot | + |
| U24685 | anti-B cell autoantibody lgM heavy chain variable V-D-J region | + |
| U89336 | Notch 4 | + |
| S68874 | EP3 prostanoid receptor EP3-l; Also: D86096_1, | + |
| HG1140-HT4817 | Collagen, Type Vi, Alpha 2; Also: M34570 | − |
| U68233 | farnesol receptor HRR-1 (HRR-1) | + |
| X95735 | zyxin 2 | − |
| D83779 | Hypothetical protein/KIAA0195 | + |
| D83174 | collagen binding prot 2 (SERPINH1); Also: X61598 | − |
| M62324 | modulator recognition factor I (MRF-1) | − |
| L04953 | Amyloid precursor protein binding APBA1 | + |
| M80899 | novel protein desmoyokin (AHNAK) | + |
| U90911 | clone 23652 sequence | − |
| Y09980 | HOXD3 | − |
| Z18956 | taurine transporter (SLC6A6) | + |
| D50926 | Hypothetical protein/KIAA0136 | + |
| U51561 | cosmid N79E2 sequence | + |
| D86965 | Hypothetical protein/KIAA0210 | + |
| HG4638-HT5050 | Spliceosomal prot Sap 49 | + |
| L16842 | ubiquinol cytochrome-c reductase core I prot (UQCRC1) | − |
| U27768 | RGP4 | + |
| AF002224 | E6-AP ubiquitin prot ligase 3A (UBE3A) | + |
| AF007111 | MDM2-like p53-binding prot (MDMX) | + |
| U41740 | trans-Golgi p230 (GOLGA4) | − |
| U70862 | nuclear factor I/B (NFIB) | + |
| D88613 | HGCMa/glial cells missing homolog 1 (GCM1) | + |
| U82979 | Ig-like transcript-3 (LILRB4) | + |
| HG4321-HT4591 | Ahnak-Related prot | + |
| U49395 | ionotropic ATP receptor P2X5a | + |
| D25217 | Membrane protein MLC1/KIAA0027 | + |
| U82535 | fatty acid amide hydrolase (FAAH) | + |
| L42611 | keratin 6 isoform K6e (KRT6E) | + |
| HG4094-HT4364 | Transcription Factor Lsf-ld; Also: U03494 | + |
| M65290 | Interleukin 12p40 (IL12p40/IL12B) | + |
| M12759 | Ig J chain (IGJ) | + |
| HG4662-HT5075 | Omega Light Chain Ig Lambda Light Chain Related | + |
| D28114 | myelin-associated oligodendrocytic basic protein (MOBP) | + |
| M81758 | voltage-dependent sodium channel SKM1 (SCN4A) | + |
| X89101 | Fas/APO1 (TNFRSF6) | + |
| U09851 | zinc finger prot (ZNF148); Also: L04282 | + |
| U33921 | HSU33921 cDNA | + |
| D30758 | Centaurin beta 1 (CENTB1)/KIAA0050 | + |
| S75578 | 4-aminobutyrate aminotransferase (ABAT) | − |
| D26067 | Hypothetical protein/KIAA0033 | + |
| D63875 | TPR-containing SH2-binding phosphoprotein/KIAA0155 | + |
| M30269 | nidogen | + |
| Y08319 | kinesin-2 (KIF2) | + |
| U33920 | clone lambda 5 semaphorin 3F (SEMA3F) | + |
| Z37166 | BAT1 nuclear RNA helicase (DEAD family) | − |
| Z11502 | annexin A13 (ANXA13) | + |
| L40395 | (clone S20iii15) | + |
| Y07701 | aminopeptidase | + |
| Y10514 | CD152 prot (CTLA4); Also: Y10508 | − |
| L14922 | DNA-binding prot (PO-GA) | + |
| HG4272-HT4542 | Hepatocyte Growth Factor Receptor | + |
| U82671 | HSP1-A from cosmids from Xq28 | + |
| X82240 | T cell leukemia/lymphoma 1 (TCL1A) | + |
| U05237 | fetal Alz-50-reactive clone 1 (FAC1) | + |
| AF006087 | Arp2/3 prot complex subunit p20-Arc (ARC20) | + |
| J04617 | elongation factor EF-1-alpha; Also: M27364 | + |
| U72512 | B-cell receptor associated prot (hBAP)//U72512 | + |
| U37219 | cyclophilin-like prot CyP-60 | + |
| HG3214-HT3391 | Metallopanstimulin 1 | + |
| X53296 | IRAP; Also: X64532_rna1, X52015 | + |
| X13255 | dopamine beta-hydroxylase type a (EC 114171) | + |
| U34380 | prot tyrosine kinase TEC and TXK; Also: D29767 | + |
| U58033 | myotubularin related prot 2 (MTMR2)/U58033 | + |
| AB002315 | Hypothetical protein/KIAA0317 | + |
| U52077 | mariner1 transposase/U52077; Also: U80776 | − |
| X77383 | cathepsin-O | + |
| J04809 | Cytosolic adenylate kinase AK1 | + |
| U89012 | dentin matrix acidic phosphoprot 1 (DMP1) | + |
| U57341 | neurofilament L (NFL) | + |
| X74330 | DNA primase (PRIM1) | + |
| Z47038 | microtubule-associated prot 1A (MAP1A)/Z47038/U38291 | + |
| L20860 | glycoprotein Ib beta (GP1BB) | + |
| U37431 | HOX A1 | + |
| U15173 | Nip2 (NIP2) | + |
| X90846 | mixed lineage kinase 2 (MAP3K10) | + |
| Z50194 | PQ-rich prot | + |
| U89922 | lymphotoxin beta isoform variant; Also: L11016_rna1, L11015 | + |
| D87684 | UBX domain-containing 2 protein/KIAA0242 | + |
| HG4258-HT4528 | Kinase Inhibitor P27kip1 Cyclin-Dependent | + |
| M31642 | hypoxanthine phosphoribosyltransferase (HPRT) | − |
| HG2992-HT5186 | Beta-Hexosaminidase Alpha Polypeptide | + |
| J03798 | autoantigen small nuclear ribonucleoprot Sm-D | + |
| X12953 | ab2 , YPT1-related and member of ras family | + |
| HG4128-HT4398 | Anion Exchanger 3 Cardiac Isom | + |
| Y10517 | CD108 prot/Y10517 | + |
| S81957 | BMP-5=bone morphogenic prot-5/S81957 | + |
| X13589 | aromatase (estrogen synthetase) (CYP19A1) | + |
| X16665 | HOX2H from the Hox2 locus | + |
| X57809 | rearranged Ig lambda light chain; Also: S42404 | + |
| S72487 | Platelet-derived endothelial growth factor 1 (ECGF1) | + |
| D42040 | RING3 protein/KIAA9001; Also: X62083, M80613 | + |
| D87078 | Translational repressor Pumilio/KIAA0235 | + |
| Z80780 | H2B/h/Z80780 | + |
| X58199 | adducin 2 beta (ADD2); Also: S81083_1 | + |
| U33822 | tax1-binding prot TXBP181, MAD1-like 1 (MAD1L1) | + |
| D63160 | DNA lectin P35/Ficolin 2 (FCN2) | + |
| U06454 | AMP-activated prot kinase (hAMPK) | + |
| Y10936 | hypothetical prot downstream of DMPK and DMAHP | + |
| HG3748-HT4018 | Basic Transcription Factor 44 Kda Subunit | + |
| U07919 | aldehyde dehydrogenase 6 | + |
| U40705 | telomeric repeat binding factor (TRF1) | − |
| U31930 | deoxyuridine nucleotidohydrolase | − |
| S78798 | 1-phosphatidylinositol-4-phosphate 5-kinase isoform C/U14957 | + |
| M95623 | hydroxymethylbilane synthase | − |
| U55206 | gamma-glutamyl hydrolase (hGH) | − |
| AB002559 | hunc18b2 | + |
| X07203 | CD20 receptor (S7) | + |
| D10495 | protein kinase C delta-type (PRKCD) | + |
| D42138 | Phosphatidylinositol glycan type B (PIG-B) | + |
| S80905 | PRB2 (PRB2L CON1+)=Con1; Also Similar To: U22312 | + |
| X52011 | MYF6 encoding a muscle determination factor | + |
| U12779 | MAP kinase activated protein kinase 2 (MAPKAPK2) | + |
| M31899 | DNA repair helicase (ERCC3) | − |
| X69141 | squalene synthase | − |
| L42572 | p87/89 mitochondrial inner membrane protein (IMMT) | − |
| M55621 | N-acetylglucosaminyltransferase I (GIcNAc-TI) | + |
| HG884-HT884 | Oncogene E6-Ap, Papillomavirus; Also: U84404 | − |
| Z35093 | SURF-1 | − |
| D16593 | BDR-2 hippocalcin | + |
| X66142 | rod cGMP phosphodiesterase 6b (PDE6B); Also: S41458 | + |
| D38583 | calgizzarin | + |
| L13329 | iduronate-2-sulfatase (IDS) | + |
| X54871 | ras-related prot RAB5B | + |
| D82344 | NBPhox (PHOX2B) | + |
| U49973 | tigger 1 transposable element | + |
| X80907 | phosphatidyl-inositol-3-kinase p85 (PIK3R2) | + |
| L13434 | chromosome 3p211 sequence | + |
| D50919 | TRIM14 protein/KIAA0129 | + |
| U10686 | MAGE-11 antigen (MAGE11) | + |
| U62389 | cytosolic NADP-dependent isocitrate dehydrogenase/U62389 | + |
| J00129 | fibrinogen beta-chain | + |
| M31523 | transcription factor (E2A) | − |
| M89957 | B cell receptor complex cell surface glycoprot (IGB) | + |
| U43030 | cardiotrophin-1 (CTF1) | + |
| X05345 | histidyl-tRNA synthetase (HRS) | − |
| U15642 | transcription factor E2F-5; Also: U31556 | + |
| U38175 | HuR RNA binding prot (HuR) | − |
| J02611 | apolipoprot D | + |
| HG3033-HT3194 | Spliceosomal prot Sap 62 | + |
| HG627-HT5098 | Rhesus (Rh) Blood Group Ce-Antigenl, 3, Rhviii; Also: X63097 | − |
| D86960 | Hypothetical protein/KIAA0205 | + |
| M13207 | granulocyte-macrophage colony-stimulating factor (CSF1) | − |
| J04101 | NAD(P)H: menadione oxidoreductase; Also: M81600 | + |
| HG3104-HT3280 | Serine Protease Met1 | + |
| HG3546-HT3744 | Pre-Splicing Factor Sf2p33; Also: HG3546-HT3746, M72709 | + |
| HG417-HT417 | Cathepsin B; Also: L22569 | − |
| D59253 | NCBP interacting prot 1 | + |
| HG4114-HT4384 | Olfactory Receptor Or17-209 | + |
| X56741 | rab8 | + |
| U58034 | myotubularin related prot 3 (MTMR3)/U58034 | − |
| X89430 | methyl CpG binding prot 2 | + |
| L24470 | prostanoid FP receptor | + |
| D38449 | G protein-coupled receptor (GPR) | + |
| U77735 | pim-2 protoonco homolog pim-2h | + |
| U07550 | Chaperonin 10 (HSPE1) | − |
| U57094 | small GTP-binding prot | + |
| D87119 | cancellous bone osteoblast | + |
| X60655 | EVX1 homeobox | + |
| X90857 | (−14) containing globin regulatory element (CGTHBA) | − |
| U21049 | DD96 | + |
| X63422 | delta-subunit of mitochondrial F1F0 ATP-synthase (clone #1) | − |
| D87076 | Br140/KIAA0239 | + |
| U50079 | histone deacetylase HD1; Also: D50405 | + |
| L10838 | SR prot family pre-splicing factor (SRp20) | + |
| X87176 | 17-beta-hydroxysteroid dehydrogenase | + |
| Z78289 | (clone 1D2)/Z78289 | + |
| U80040 | aconitase nuclear encoded mitochondrial prot | − |
| X76105 | Death-associated protein (DAP1) | − |
| D85418 | phosphatidylinositol-glycan-class C (PIGC) | + |
| X17648 | granulocyte-macrophage colony-stimulating factor receptor | + |
| X65488 | Heterogeneous nuclear ribonucleoprotein U (HNRPU) | + |
| Z97074 | Rab9 effector p40/Z97074 | − |
| M29994 | alpha-I spectrin (SPNA1); Also: M61877, M61826 | + |
| U22398 | Cdk-inhibitor p57KIP2 (KIP2) | − |
| M14123 | HERV-K10 neutral protease | − |
| X59871 | TCF-1 T cell factor 1 | + |
| M77349 | transforming growth factor-beta induced product (BIGH3) | − |
| M25809 | endomembrane proton pump subunit | + |
| U28369 | semaphorin V | − |
| HG3597-HT3800 | Major Histocompatibility Complex, Class I X12432 | + |
| AF001359 | mismatch repair prot (hMLH1)/AF001359 | − |
| D14661 | Splicing regulator WTAP protein/KIAA0105 | + |
| M58285 | membrane-associated prot (HEM-1) | + |
| M58583 | precerebellin and cerebellin | − |
| M80397 | DNA polymerase delta catalytic subunit; Also: M81735 | − |
| X63679 | TRAMP prot | + |
| X72964 | caltractin | − |
| U49188 | placenta (Diff33) | + |
| X57398 | pM5 prot | − |
| X76061 | p130 retinoblastoma-like 2 (RBL2) | + |
| M26901 | renin; Also: L00073 | + |
| U34976 | gamma-sarcoglycan (SGCG) | − |
| U31903 | CREB-RP (CREBL1); Also: U89337_1, X98054 | + |
| S90469 | cytochrome P450 reductase | − |
| D90276 | CGM7 nonspecific cross-reacting antigen (NCA) | + |
| S81243 | CHN=steroid/thyroid orphan receptor homolog; Also: U12767 | + |
| HG2602-HT2698 | Succinate Dehydrogenase Flavoprotein (HSSUCCDH) | + |
| M22995 | ras-related prot (Krev-1) | + |
| X01630 | argininosuccinate synthetase | − |
| D83243 | NPAT | + |
| U20938 | Lymphocyte dihydropyrimidine dehydrogenase (DPYD) | + |
| J05243 | nonerythroid alpha-spectrin (SPTAN1) | − |
| M34175 | beta adaptin | + |
| X67235 | proline rich homeobox (Prh) prot; Also: L16499 | + |
| U05040 | FUSE binding protein (FUBP1) | + |
| D82061 | short-chain alcohol dehydrogenase family | + |
| U91932 | AP-3 complex sigma 3A subunit (AP3S1) | − |
| L32832 | alpha fetoprotein enhancer binding protein/D10250 | − |
| U37352 | prot phosphatase 2AB alpha 1 | + |
| J03764 | Plasminogen activator inhibitor 1 | + |
| D86971 | Hypothetical protein/KIAA0217 | + |
| D29805 | beta-14-galactosyltransferase | + |
| X79353 | XAP-4 GDP-dissociation inhibitor (GDI1) | − |
| U57057 | WD prot IR10 | + |
| M16750 | pim-1 oncogene; Also: M27903, M24779, M54915 | + |
| U51903 | RasGAP-related prot (IQGAP2) | + |
| M54995 | connective tissue activation peptide III | + |
| U83410 | CUL-2 (cul-2) | + |
| U15174 | Nip3 (NIP3) | + |
| L19183 | MAC30 | − |
| X96506 | NC2 alpha subunit; Also: U41843 | + |
| M33308 | Vinculin (VCL) | − |
| HG3635-HT3845 | Zinc Finger prot, Kruppel-Like | + |
| Z48042 | encoding GPI-anchored prot p137 | + |
| HG1879-HT1919 | Ras-Like prot Tc10 | + |
| M73548 | polyposis locus (DP25) | + |
| X06389 | synaptophysin (p38) | + |
| U70439 | silver-stainable prot SSP29; Also: Y07570 | − |
| D67029 | SEC14L | + |
| U82279 | Ig-like transcript 2 (LILRB1) | + |
| HG3925-HT4195 | Surfactant prot Sp-A2 Delta; Also: M30838, M68519_rna1 | + |
| U61234 | tubulin-folding cofactor C | + |
| J02963 | platelet glycoprot IIb | + |
| D82345 | NB thymosin beta | − |
| L10615 | beta casein (CSN2); Also: X17070 | + |
| D17532 | RCK | + |
| D87436 | Lipin 2/KIAA0249 | + |
| X13967 | leukaemia inhibitory factor (LIF/HILDA) | + |
| M91036 | G-gamma globin (HBG2) | + |
| D63506 | unc-18 homolog | + |
| M61733 | erythroid membrane protein 41 (EPB41) | + |
| HG4102-HT4372 | N-Ethylmaleimide-Sensitive Factor (NSF) | + |
| X07173 | second prot of inter-alpha-trypsin inhibitor complex | + |
| U14391 | myosin-IC | − |
| D88795 | cadherin | + |
| M21154 | S-adenosylmethionine decarboxylase | + |
| D16217 | Calpastatin (CAST) | + |
| M91029 | AMP deaminase (AMPD2) | + |
| J04444 | cytochrome c-1 (CYC1) | − |
| X16983 | integrin alpha-4 subunit VLA4 (ITGA4) | + |
| D29642 | GTPase/KIAA0053 | + |
| D42108 | phospholipase C-like 1 (PLCL1) | + |
| M19045 | lysozyme | + |
| U69546 | RNA binding prot ETR3 | + |
| U24704 | antisecretory factor-1 (PSMD4) | − |
| X86809 | major astrocytic phosphoprot PEA-15 | − |
| M13929 | c-myc-P64; Also: HG3523-HT4900, HG3523-HT4899, L00058 | + |
| X16546 | eosinophil derived neurotoxin | + |
| U48296 | prot tyrosine phosphatase PTPCAAX1 (hPTPCAAX1) | − |
| Z48923 | BMPR-II | + |
| Y10055 | phosphoinositide 3-kinase | + |
| L06175 | P5-1 | + |
| M27504 | topoisomerase type II (Topo II)/M27504/Also: Z15115 | + |
| U23946 | putative tumor suppressor (LUCA15) | + |
| X80818 | metabotropic glutamate receptor type 4 | + |
| U80628 | thymidine kinase 2 isom B (TK2) alternatively spliced | − |
| U14973 | ribosomal prot S29 | + |
| M38258 | retinoic acid receptor gamma 1 | + |
| D83597 | RP105 | + |
| M81830 | somatostatin receptor isom 2 (SSTR2) | + |
| Z37976 | latent transming growth factor-beta binding prot (LTBP-2) | − |

TABLE 12
Least discriminatory genes for MS (regardless of treatment
with Avonex or not) vs. Healthy Donors and ALS

| | | |
|---|---|---|
| L33881 | prot kinase C iota isom | + |
| L14778 | calmodulin-dependent prot phosphatase PPP3CA | + |
| D45371 | GS3109 adipose most abundant gene transcript 1 (APM1) | + |
| X53683 | LAG-1 | + |
| D87442 | Nicastrin/KIAA0253 | + |
| X52730 | phenylethanolamine n-methyltransferase (PNMT) | + |
| M95549 | sodium/glucose cotransporter-like protein (SLC5A2) | + |
| AF005775 | caspase-like apoptosis regulatory prot 2 (CLARP) | + |
| Z69043 | translocon-associated prot delta subunit precursor (SSR4) | − |
| X65857 | HGMP07E olfactory receptor | + |
| U09953 | ribosomal prot L9 | − |
| X76648 | glutaredoxin | + |
| U20760 | extracellular calcium-sensing receptor | + |
| M22324 | aminopeptidase N | + |
| Z19554 | vimentin (VIM); Also: M18895_2 | − |
| AJ001421 | Rer1 | + |
| U00928 | clone CE29 4.1 (CAC)n/(GTG)n repeat-containing | + |
| M77144 | 3-b-hydroxysteroid dehydrogenase/5-delta-4-delta isomerase | + |
| S69265 | neuron-specific RNA recognition motifs; Also: L26405 | + |
| U55054 | K-Cl cotransporter (hKCC1) | + |
| M92642 | alpha-1 type XVI collagen (COL16A1), | + |
| U47686 | signal transducer and activator of transcription Stat5B/U48730 | + |
| HG1471-HT3923 | Transcription Factor Oct-1a/1b; Also: X13403 | − |
| L06845 | cysteinyl-tRNA synthetase | + |
| D63998 | golgi alpha-mannosidase II (MAN2A1) | + |
| AF002700 | TGF-beta related neurotrophic factor receptor 2 (TRNR2) | + |
| Y13618 | DFFRY prot | + |
| M19283 | cytoskeletal gamma-actin | − |
| X13930 | CYP2A4 P-450 IIA4 prot | + |
| U07857 | 18 kDa Alu RNA binding prot | − |
| D86957 | Septin-like protein/KIAA0202 | − |
| HG907-HT907 | Mg44 | + |
| X16316 | vav oncogene | + |
| M57609 | DNA-binding prot (GLI3) | + |
| U58032 | myotubularin related prot 1 (MTMR1)/U58032 | + |
| U68485 | Box-dependent MYC-interacting prot-1 (BIN1) | + |
| U82130 | tumor susceptiblity prot (TSG101) | − |
| X95404 | non-muscle type cofilin | − |
| M87507 | interleukin-1 beta convertase (IL1BCE); Also: U13697 | + |
| HG3405-HT3586 | Zinc Finger prot Hzf3 | + |
| X01677 | glyceraldehyde-3-phosphate dehydrogenase | − |
| U07563 | ABL and putative M8604 Met prot (M8604 Met) | − |
| D63879 | T cell-recognized SART-3/KIAA0156 | + |
| L07261 | alpha adducin (ADD1) | + |
| HG2479-HT2575 | Helix-Loop-Helix prot Sef2-1d; Also: M74719 | + |
| HG3925-HT4195 | SFTPA2D | + |
| X17622 | HBK2 potassium channel prot | + |
| HG3884-HT4154 | Homeotic protein HPX42 | − |
| U04270 | putative potassium channel subunit (KCNH2) | − |
| Y00285 | insulin-like growth factor II receptor; Also: J03528 | − |
| X99479 | NK receptor, clone 12.11C-Also Similar To: X93596, L76672 | − |
| HG1602-HT1602 | Utrophin | + |
| U67171 | selenoprot W (selW)/U67171 | − |
| L20941 | ferritin heavy chain (FTH1) | − |
| U34877 | biliverdin-IXalpha reductase | − |
| U79255 | X11 prot | − |
| D49490 | disulfide isomerase-related protein (PDIR) | + |
| Y07827 | butyrophilin (BTN)/U90552 | + |
| X82693 | Lymphocyte antigen 6 complex, locus D (E48) | + |
| L20815 | S protein/corneodesmosin (CDSN) | + |
| U56102 | adhesion molecule DNAM-1 | + |
| U15306 | cysteine-rich sequence-specific DNA-binding prot NFX1 | + |
| X79683 | Z68155 and others | − |
| D31888 | CoREST protein (RCOR)/KIAA0071 | + |
| M37721 | peptidylglycine alpha-amidating monooxygenase | − |
| D38024 | facioscapulohumeral muscular dystrophy (FSHD) | + |
| D26561 | ORF E7 from papillomavirus 5b genome | + |
| D56495 | Reg-related sequence derived peptide-2 (REGL) | + |
| D63485 | Inhibitor of NFkB kinase epsilon subU. lkBKE/KIAA0151 | + |
| Y14140 | G protein-encoding beta 3 subunit 1 (GNB3) | + |
| L20298 | TRANSCRIPTION FACTORS (CBFB) | + |
| U46025 | translation initiation factor elF-3 p110 subunit | − |
| D49728 | NAK1 DNA binding prot, | + |
| AB002365 | Hypothetical protein/KIAA0367 | − |
| HG1733-HT1748 | Moloney Murine Sarcoma Viral Oncogene Homolog | + |
| M54915 | h-pim-1 prot (h-pim-1); Also: M27903, M24779 | + |
| U23435 | Abl interactor 2 (Abi-2); Also: X95632, X95677 | + |
| X17567 | snRNP prot B; Also: J04564 | − |
| D50525 | TI-227H/D50525 | + |
| D50683 | TGF-betaIIR alpha | + |
| U41804 | putative T1/ST2 receptor binding prot precursor | − |
| U71364 | serine protase inhibitor (P19) | + |
| X71125 | glutamine cyclotransferase (QPCT) | + |
| D21235 | HHR23A prot | − |
| U35113 | metastasis-associated mta1 | − |
| L42373 | prot phosphatase 2A B56-alpha | + |
| M30818 | interferon-induced cellular resistance mediator MxB (MX2) | + |
| X61373 | alternatively spliced tau/X61373 | + |
| U46751 | phosphotyrosine independent ligand p62 | − |
| Z18951 | caveolin | − |
| M93221 | macrophage mannose receptor (MRC1) | − |
| M63838 | interferon-gamma induced prot (IFI 16); Also: S75433 | + |
| X62654 | ME491/CD63 antigen | − |
| K02574 | purine nucleoside phosphorylase (PNP) | + |
| HG1103-HT1103 | Guanine Nucleotide-Binding prot Ral | + |
| X12447 | aldolase A (ALDOA) (EC 41213) | − |
| X95826 | ART4/X95826 | + |
| D78514 | ubiquitin-conjugating enzyme | + |
| M76424 | carbonic anhydrase VII (CA VII) | + |
| D12775 | erythrocyte-specific AMP deaminase; Also: U29926_2 | + |
| U83192 | post-synaptic density prot 95 (PSD95) | − |
| D87073 | zinc finger protein znf142/KIAA0236 | + |
| S50017 | 2,3-cyclic nucleotide 3-phosphodiesterase (CNP) | + |
| X07618 | cytochrome P450 db1 variant a; Also: X07619, X16866 | + |
| X67683 | keratin 4 (KRT4) | + |
| M55543 | guanylate binding prot isom II (GBP-2) | − |
| U08096 | peripheral myelin prot-22 (PMP22) non-coding 1B/U08096 | − |
| HG3141-HT3317 | Nadh-Ubiquinone Oxidoreductase, 39 Kda Subunit | + |
| L46720 | autotaxin-t (atx-t); Also: L35594 | − |
| X98248 | Sortilin (SORT1) | + |
| M13241 | N-myc | − |
| U65581 | ribosomal prot L3-like | + |
| U58334 | Bcl2 p53 binding prot Bbp/53BP2 (BBP/53BP2) | + |
| S50223 | HKR-T1 = Kruppel-like zinc finger prot | + |
| D13435 | phosphatidyl-inositol-glycan class F | + |
| L37378 | Acc# L37378 | − |
| U16307 | glioma pathosis-related prot (GliPR) | + |
| L22650 | early lymphoid activation prot (EPAG) | + |
| U49837 | LIM prot MLP (CSRP3) | + |
| M32334 | intercellular adhesion molecule 2 (ICAM-2) | + |
| U79294 | clone 23748 phosphatidic acid phosphatase 2B (PPAP2B) | + |
| X16560 | COX VIIc subunit VIIc of cytochrome c oxidase (EC 1931) | − |
| X54816 | alpha-1-microglobulin-bikunin (AMBP) | + |
| M90656 | gamma-glutamylcysteine synthetase (GCS) | + |
| HG2239-HT2324 | Potassium Channel prot Z11585 | + |
| M27394 | B-lymphocyte cell-surface antigen B1 (CD20); Also: X12530 | + |
| AC002073 | WUGSC: DJ515N12/PAC clone DJ515N1/22q112-q22 | + |
| M17733 | thymosin beta-4 | + |
| S78432 | transmembrane 4 prot/S78432 | − |
| S72904 | APK1 antigen = MAb KI recognized | − |
| M80629 | cdc2-like protein kinase 5 (CDC2L5) | + |
| U89335 | Notch 4 | + |
| L34657 | platelet/endothelial cell adhesion molecule-1 (PECAM-1) | + |
| J00123 | enkephalin | − |
| X70944 | PTB-associated splicing factor; Also: X16850 | + |
| X80909 | alpha NAC nascent polypeptide-associated complex | − |
| L37042 | casein kinase I alpha isom (CSNK1A1) | + |
| X05196 | aldolase C | + |
| U40215 | synapsin IIb | + |
| D87460 | paralemmin/KIAA0270 | + |
| M37435 | macrophage-specific colony-stimulating factor (CSF-1) | + |
| M94046 | zinc finger prot (MAZ) | − |
| X69433 | mitochondrial isocitrate dehydrogenase (NADP+) | − |
| U80017 | Survival motor neuron protein (SMN) | − |
| M33336 | cAMP-dependent prot kinase type I-alpha subunit (PRKAR1A) | + |
| D38503 | PMS8 (yeast PMS1 homolog | + |
| HG3123-HT3299 | Homeotic prot Gbx2 | + |
| Y08302 | MAP kinase phosphatase 4 | − |
| L10405 | DNA binding prot surfactant prot B/L 10405 | − |
| X80026 | B-cam | + |
| X59932 | C-SRC-kinase; Also: X60114 | + |
| Y10812 | fructose-bisphosphatase | + |
| U79295 | clone 23961 sequence | + |
| X51804 | PMI a putative receptor prot | − |
| U23430 | Cholecystokinin type A receptor (CCKAR); Also: L19315 | + |
| M28879 | granzyme B/CTLA-1 (GZMB) | + |
| L15326 | endoperoxide synthase type II; Also: D28235, U04636_rna1 | + |
| L32977 | ubiquinol cytochrome c reductase Rieske iron-sulphur protein | − |
| D50925 | serine-threonine protein kinase/KIAA0135 | + |
| U28049 | TBX2 (TXB2) | − |
| M29277 | isolate JuSo MUC18 glycoprot (3′ variant) | − |
| U88726 | symplekin/U88726 | + |
| U63541 | expressed in HC/HCC livers and MolT-4 proliferating cells | + |
| U80811 | Lysophosphatidic acid receptor homolog (EDG2) | − |
| U02683 | Nuclear respiratory factor 1 (NRF1); Also: L22454, U18383 | + |
| S75463 | P43 = mitochondrial elongation factor homolog | − |
| D31767 | DAZ associated protein 2/KIAA0058 | + |
| HG2320-HT2416 | Integrin Beta 3 Subunit | + |
| HG3514-HT3708 | Tropomyosin Tm30nm Cytoskeletal | + |
| U40714 | tyrosyl-tRNA synthetase (YARS) | + |
| D16181 | PMP2 | + |
| D79999 | VPARP vault protein/KIAA0177 | + |
| M35999 | platelet glycoprot IIIa/Integrin beta 3 (ITGB3) | + |
| M21186 | neutrophil cytochrome b light chain p22 | + |
| M28713 | NADH-cytochrome b5 reductase (b5R) | − |
| U59309 | fumarase precursor (FH) nuclear encoding mitochondrial prot | − |
| U64863 | HPD-1 (hPD-1) | − |
| HG4194-HT4464 | Sodium/Hydrogen Exchanger 5 | + |
| Z83336 | hH2B/d | + |
| D50402 | Natural resistance associated macrophate prot1 (NRAMP1) | + |
| J04088 | DNA topoisomerase II (top2) | + |
| X65644 | MBP-2 MHC binding prot 2 | + |
| M31627 | X box binding prot-1 (XBP-1) | + |
| L06633 | TRANSCRIPTION FACTORS | + |
| D50550 | Lethal giant larvae homolog 1 (LLGL1) | + |
| U25265 | MEK5 | + |
| J05614 | proliferating cell nuclear antigen (PCNA) | + |
| X13451 | HG2563-HT2659 Same Uni Cluster as U05259 | − |
| U10868 | aldehyde dehydrogenase ALDH7 | + |
| L75847 | Zinc finger prot 45 (ZNF45) | + |
| HG644-HT644 | Histone H1.1 (HIST1H1A) | − |
| Z84721 | cosmid from 16p13.3 Contains alpha and zeta globin | + |
| HG2149-HT2219 | Mucin/M57417 | + |
| L21936 | succinate dehydrogenase flavoprot subunit (SDH) | − |
| U01212 | olfactory marker prot (OMP) | + |
| U14193 | TFIIA gamma subunit | − |
| X68486 | A2a adenosine receptor | + |
| Z48633 | retrotransposon | − |
| D87685 | TFIIS-like PHD finger protein 3/KIAA0244 | + |
| U18062 | TFIID subunit TAFII55 (TAFII55) | − |
| X95808 | prot encoded by DXS6673E, for mental retardation/AB002383 | + |
| U38276 | semaphorin III family homolog (SEMA3F) | − |
| M64497 | apolipoprot Al regulatory prot (ARP-1) | − |
| U79526 | orphan G-prot coupled receptor Dez isoform a | − |
| HG4167-HT4437 | Nuclear Factor 1 A Type | + |
| M31520 | Ribosomal S24 | − |
| U00238 | glutamine PRPP amidotransferase (GPAT) | − |
| K02777 | T-cell receptor active alpha-chain from Jurkat cell line/M12959 | + |
| L22342 | nuclear phosphoprot | + |
| U58087 | Hs-cul-1 | + |
| X81892 | G protein-couped receptor 64 (GPR64) | + |
| U70323 | ataxin-2 (SCA2) | − |
| S77812 | flt = vascular endothelial growth factor receptor/VEGF receptor | + |
| D50918 | septin 2, 6 (SEPT6)/KIAA0128 | + |
| X13839 | vascular smooth muscle alpha-actin | − |
| S76617 | protein tyrosine kinase (BLK) | + |
| HG36-HT4101 | PM-Sc1 autoantigen/M58460 | − |
| U12535 | epidermal growth factor receptor kinase substrate (EPS8) | − |
| X80590 | PHKG1 | + |
| M58028 | ubiquitin-activating enzyme E1 (UBE1) | − |
| D13634 | Hypothetical protein/KIAA0009 | + |
| M68891 | GATA-binding prot (GATA2) | + |
| AB000449 | VRK1 | + |
| S73885 | AP-4 = basic helix-loop-helix DNA-binding prot; Also: X57435 | − |
| U07807 | metallothionein IV (MTIV) | + |
| U95626 | ccr5 | + |
| L13977 | prolylcarboxypeptidase (PRCP) | − |
| J05068 | transcobalamin I | + |
| U71087 | MAP kinase kinase MEK5b | + |
| M57763 | ADP-ribosylation factor (hARF6) | + |
| M98528 | neuron-specific prot last clone D4S234 | + |
| U67963 | lysophospholipase homolog (HU-K5) | − |
| M20218 | coagulation factor Xl | + |
| U93049 | SLP-76 associated prot | + |
| HG1102-HT1102 | Ras-Related C3 Botulinum Toxin Substrate (RAC1) | − |
| HG1428-HT1428 | Globin, Beta; Also: U01317_6 | + |
| M94172 | N-type calcium channel alpha-1 subunit (CACNA1B) | + |
| U81556 | hypothetical prot A4 | + |
| M80244 | E16 | + |
| L40387 | thyroid receptor interactor TRIP14 (OASL) | + |
| U67932 | cAMP phosphodiesterase (Pde7A2)/U67932; Also: L12052 | + |
| U68494 | Hbc647 sequence | + |
| U01317 | Beta-globin thalassemia | + |
| HG2705-HT2801 | Serine/Threonine Kinase Z25427-Also: X97630 | + |
| M63573 | secreted cyclophilin-like prot (SCYLP) | − |
| D86959 | hSLK serine threonine kinase/KIAA0204 | + |
| HG3570-HT3773 | prot Phosphatase Inhibitor Homolog | − |
| U80457 | TRANSCRIPTION FACTORS SIM2 short form | − |
| U97018 | echinoderm microtubule-associated prot homolog HuEMAP | − |
| U90547 | Ro/SSA ribonucleoprot homolog (RoRet) | − |
| HG821-HT821 | Ribosomal prot S13 | + |
| Z29505 | nucleic acid binding prot sub23 | + |
| X84213 | BAK BCI-2 homolog; Also: U16811, U23765 | + |
| D28235 | PTGS2 prostaglandin endoperoxide synthase-2/U04636_rna1 | + |
| L33842 | type II inosine monophosphate dehydrogenase (IMPDH2) | − |
| U30521 | P311 HUM -31 | − |
| X64330 | ATP-citrate lyase | − |
| X15341 | COX Vla-L cytochrome c oxidase liver-specific subunit Via | − |
| U82010 | heme A: farnesyltransferase (COX10) | − |
| X73478 | hPTPA | − |
| D32001 | serum amyloid A1 gamma | + |
| X03342 | ribosomal prot L32 | − |
| HG3117-HT3293 | Mps1 | + |
| U65093 | Msg1-related 1 (mrg1) | + |
| U28831 | prot immuno-reactive with anti-PTH polyclonal antibodies | + |
| U41766 | metalloprotease/disintegrin/cysteine-rich prot precursor MDC9 | − |
| Z49269 | chemokine HCC-1 (CCL14) | + |
| S65738 | Actin depolymerizing factor | − |
| X56692 | C-reactive prot | + |
| U52522 | arfaptin 2 putative target prot of ADP-ribosylation factor | − |
| X64364 | M6 antigen | − |
| Z22548 | thiol-specific antioxidant peroxiredoxin 2 (PRDX2) | − |
| U04806 | U03858 and others | + |
| L34059 | cadherin-4 | − |
| M21188 | insulin-degrading enzyme (IDE) | − |
| L34060 | cadherin-8 | + |
| HG4704-HT5146 | Glial Growth Factor 2 | + |
| M73077 | glucocorticoid receptor repression factor 1 (GRF-1) | − |
| M32011 | neutrophil oxidase factor (p67-phox) | + |
| HG3914-HT4184 | Cell Division Cycle prot 2-Related prot Kinase Pisslre/X78342 | + |
| HG1783-HT1803 | Islet Amyloid Polypeptide; Also: X81832 | + |
| X53416 | Actin-binding prot (filamin) (ABP-280) | − |
| Z84497 | chrom 6 cosmid contains RING3, CpG Island; Also: Z96104_1 | + |
| X63131 | My1 (PML); Also: M73778 | − |
| HG2815-HT2931 | Unknown function prot | − |
| U90913 | clone 23665 sequence | − |
| U70735 | 34 kDa mov34 isologue/U70735 | − |
| Y00062 | T200 leukocyte common antigen (CD45 LC-A) | + |
| L04270 | (clone CD18) tumor necrosis factor receptor 2 related prot | − |
| J02854 | 20-kDa myosin light chain (MLC-2) | − |
| U37408 | C-terminal binding protein 1 (CTBP1) | − |
| U48736 | serine/threonine-prot kinase PRP4h (PRP4h) | + |
| X12530 | B lymphocyte antigen CD20 (B1f, Bp35); Also: X07203 | + |
| M83738 | prot-tyrosine phosphatase (PTPase MEG2) | + |
| X15331 | phosphoribosylpyrophosphate synthetase subunit one/D00860 | + |
| D13370 | APEX nuclease (APEX1) | + |
| U52700 | tenascin-X (XB) RACE clone N1/U52700 | + |
| M95809 | Basic TRANSCRIPTION FACTORS 62 kD subunit (BTF2) | + |
| HG3638-HT3849 | Amyloid Beta (A4) Precursor prot; Also: Y00264 | − |
| L14595 | alanine/serine/cysteine/threonine transporter (ASCT1) | + |
| U31342 | nucleobindin | + |
| U45880 | X-linked inhibitor of apotosis prot XIAP | + |
| S71129 | acetylcholinesterase (ACHE) | + |
| D00760 | proteasome subunit HC3 | + |
| HG3254-HT3431 | Phosphatidylinositol 3-Kinase P110 Beta | + |
| M64099 | gamma-glutamyl transpeptidase-related prot (GGT-Rel) | − |
| M98539 | prostaglandin D2 synthase | − |
| X59812 | CYP 27 vitamin D3 25-hydroxylase | + |
| D79986 | bcl-2-associated transcription factor/KIAA0164 | + |
| U57342 | myelodysplasia/myeloid leukemia factor 2 (MLF2) | − |
| X15187 | tra1 homolog of murine tumor rejection antigen gp96 | − |
| U79275 | Clone 23947 | + |
| L10338 | sodium channel beta-1 subunit (SCN1B)/U12194/L16242 | + |
| HG2247-HT2332 | Major Intrinsic prot | + |
| X16609 | ankyrin (variant 2.1); Also: HG2737-HT2837 | − |
| D50810 | placental leucine aminopeptidase | + |
| U43292 | MDS1B (MDS1) | − |
| D14664 | Lectin C domain-containing protein homolog/KIAA0022 | + |
| X98253 | ZNF183/X98253 | + |
| S75256 | HNL = neutrophil lipocalin; Also: X99133 | + |
| Z24727 | tropomyosin isoform | − |
| J02883 | Colipase (CLPS) | + |
| M83221 | I-Rel | + |
| D83260 | HXC-26; Also: D83389 | − |
| J05682 | subunit C of V-ATPase (vat C) | + |
| M15841 | U2 small nuclear RNA-associated B' antigen | − |
| D21852 | hypothetical protein/KIAA0029 | + |
| U11861 | G10 homolog (edg-2) | − |
| X72755 | Humig | − |
| L16896 | zinc finger prot | − |
| X81003 | HCG V | + |
| U37012 | cleavage and polyadenylation specificity factor | + |
| M22638 | LYL-1 prot | + |
| X52221 | ERCC2 | + |
| AF006084 | Arp2/3 prot complex subunit p41-Arc (ARC41) | + |
| M83088 | phosphoglucomutase 1 (PGM1) | − |
| D79989 | centaurin gamma-1/KIAA0167 | + |
| M75715 | TB3-1; Also: X81625 | − |
| J05582 | Mucin 1 (MUC1); Also: J05581 | − |
| L49219 | retinoblastoma susceptibility prot (RB1) deletion mutant | + |
| M93284 | pancreatic lipase related prot 2 (PLRP2) | + |
| M67468 | Fragile X mental retardation 1 FMR-1; Also: X69962, L19493 | + |
| U43965 | ankyrin G119 (ANK3) | + |
| U60060 | FEZ1 | − |
| U86136 | telomerase-associated prot TP-1 | − |
| M19267 | tropomyosin; Also: X12369 | − |
| X78686 | Chemokine (C-X-C motif) ligand 5 (CXCL5) | + |
| D28915 | hepatitis C-associated protein p44 (IFI44) | + |
| U78027 | L44L (L44-like ribosomal prot) | + |
| U71300 | snRNA activating prot complex 50 kD subunit (SNAP50) | + |
| V00565 | preproinsulin; Also: M10039 | + |
| HG2566-HT4867 | Microtubule-Associated prot Tau | + |
| D86331 | MT2-MMP matrix metalloprot; Also: Z48482 | − |
| M81933 | Cell division cycle 25A (CDC25A) | + |
| L33801 | prot kinase | − |
| S67070 | heat shock prot HSP72 homolog | − |
| M63262 | 5-lipoxygenase activating prot (FLAP) | + |
| X02761 | fibronectin (FN precursor); Also: HG3044-HT2527 | − |
| M65214 | (HeLa) helix-loop-helix prot HE47 (E2A); Also: M31523 | + |
| HG2815-HT2931 | Myosin, Light Chain/U02629; Also: HG2815-HT1357, M22919 | − |
| U18009 | chromosome 17q21 clone LF113 | − |
| D49818 | fructose 6-phosphate 2-kinase/fructose 2 6-bisphosphatase | − |
| HG2755-HT2862 | T-Plastin | − |
| X04143 | bone gla prot (BGP) | − |
| U40282 | integrin-linked kinase (ILK) | − |
| Z50022 | surface glycoprot | − |
| U22055 | 100 kDa coactivator | − |
| M94880 | MHC class I (HLA-A*8001); Also Similar To: M80469_rna1 | + |
| D38122 | Fas ligand | + |
| M93405 | methylmalonate semialdehyde dehydrogenase (ALDH6A1) | + |
| X79780 | YPT3 | − |
| U17969 | initiation factor eIF-5A | − |
| X51466 | elongation factor 2 | − |
| Z74616 | prepro-alpha2(I) collagen; Also: J03464 | − |
| D78367 | K12 keratin | + |
| L28821 | alpha mannosidase II isozyme | − |
| M29536 | translational initiation factor 2 beta subunit (eIF-2-beta) | − |
| Y07829 | RING prot | + |
| X97324 | adipophilin (ADFP) | + |
| X56494 | M M1-type and M2-type pyruvate kinase | − |
| L15533 | Pancreatitis associated prot | − |
| L15388 | G prot-coupled receptor kinase (GRK5) | − |
| U78521 | immunophilin homolog ARA9 | + |
| L07541 | replication factor C 38-kDa subunit | + |
| M25667 | neuronal growth prot 43 (GAP-43) | + |
| X82279 | Fas, Apo-1/X82279; Also: D31968 | + |
| X83490 | Fas/Apo-1/X83490; Also: X83493, X63717 | + |
| M81780 | Sphingomyelin phosphodiesterase 1 (SMPD1) | + |
| U59748 | Desert hedgehog (hDHH)/U59748/ | + |
| D42073 | reticulocalbin | − |
| L37792 | syntaxin 1A | + |
| M96233 | glutathione transferase class mu number 4 (GSTM4)/M99421 | − |
| U18919 | chromosome 17q12-21 clone pOV-2 | − |
| U09087 | thymopoietin (TMPO); Also: U09088 | + |
| X63097 | Rhesus polypeptide RhXIII | + |
| U22431 | hypoxia-inducible factor 1 alpha (HIF-1 alpha)/X72726, U29165 | − |
| X75593 | rab 13 | − |
| D63486 | hypothetical protein/KIAA0152 | − |
| M23114 | calcium-ATPase (HK1) | − |
| M17885 | Acidic ribosomal phosphoprot P0 | − |
| U28014 | cysteine protease (ICErel-II) | + |
| X84195 | acylphosphatase muscle type (MT) isoenzyme | + |
| Z80787 | Histone H4 | + |
| U58048 | metallopeptidase PRSM1 | − |
| M24899 | triiodothyronine (ear7) | − |
| M63928 | T cell activation antigen (CD27) | + |
| M55210 | laminin B2 chain (LAMB2) | − |
| HG4460-HT4729 | lg Heavy Chain Vdjc Regions | + |
| X85116 | epb72; Also: U33931 Same Unigene Cluster as M81635. | − |
| U20908 | melanoma ubiquitous mutated prot (MUM-1)/U20908 | − |
| D50532 | macrophage lectin 2 (HML2) | + |
| Z68274 | DNA sequence from Huntingtons Disease Region/Z68274 | + |
| D50915 | hypothetical protein/KIAA0125 | + |
| D38550 | E2f-3 transcription factor/KIAA0075 | + |
| M80333 | m5 muscarinic acetylcholine receptor | + |
| Z24725 | mitogen inducible mig-2 | − |
| U08815 | splicesomal prot (SAP 61) | − |
| U30313 | diadenosine tetraphosphatase (NUDT2) | − |
| D42044 | hypothetical protein/KIAA0090 | + |
| U32849 | Hou | + |
| Z31357 | cysteine dioxygenase type 1 | + |
| M95787 | 22 kDa smooth muscle prot (SM22) | − |
| X76732 | NEFA prot (DNA-binding leucine zipper Ca-binding EF-hand) | − |
| M74002 | arginine-rich nuclear prot | − |
| L35249 | vacuolar H+-ATPase Mr 56,000 subunit (HO57); Also: M60346 | + |
| U17760 | laminin S B3 chain (LAMB3) | + |
| D42053 | Site-1 protease transcription factor/KIAA0091 | + |
| D49817 | fructose 6-phosphate 2-kinase/fructose 2 6-bisphosphatase | − |
| U30255 | phosphogluconate dehydrogenase (hPGDH) | + |
| M86917 | oxysterol-binding prot (OSBP) | + |
| M16714 | MHC class I divergent lymphocyte antigen; clone RS5 | + |
| X59842 | PBX2; Also: U89336_2, D28769_1, X80700_rna1 | − |
| HG37-HT37 | HG37-HT37 | + |
| L05628 | multidrug resistance-associated prot (MRP); Also: X78338 | + |
| U38980 | PMS2 related (hPMSR6) | + |
| U27193 | prot-tyrosine phosphatase | − |
| HG3255-HT3432 | Gaba A Receptor Beta 2 Subunit | + |
| D88422 | DNA cystatin A | + |
| U59289 | H-cadherin | − |
| M12125 | fibroblast muscle-type tropomyosin, | − |
| J05016 | prot disulfide isomerase related prot (ERp72); Also: J05016 | + |
| D63478 | Hypothetical protein/KIAA0144 | − |
| D29954 | hypothetical protein/KIAA0056 | + |
| X06318 | protein kinase C (PKC) type beta I (PRKCB) | + |
| L35240 | enigma | − |
| U49070 | peptidyl-prolyl isomerase and essential mitotic regulator PIN1 | − |
| L10413 | farnesyltransferase alpha-subunit | − |
| X82634 | hair keratin acidic 3B (KRTHA3B) | + |
| M91463 | glucose transporter (GLUT4) | + |
| L19779 | histone H2A2 | + |
| S69790 | Brush-1 = tumor suppressor | + |
| D55640 | monocyte pseudoautosomal boundary-like sequence | − |
| D49357 | S-adenosylmethionine synthetase | + |
| U73304 | CB1 cannabinoid receptor (CNR1) . | + |
| U52154 | G prot-coupled inwardly rectifying potassium channel Kir34 | + |
| Z23090 | 28 kDa heat shock prot | − |
| S76756 | 4R-MAP2 = microtubule-associated prot 2 4R isoform/S76756 | + |
| X86018 | MUF1 prot | + |
| X97444 | transmembrane prot Tmp21-IIex. /X97444 | + |
| U35234 | prot tyrosine phosphatase sigma | + |
| U41515 | deleted in split hand/split foot 1 (DSS1) | − |
| U58970 | outer mitochondrial membrane translocase (TOMM34) | − |
| D49824 | HLA-B null allele; Also: L42345 and others | + |
| L11672 | Kruppel related zinc finger prot (HTF10) | + |
| M16336 | T-cell surface antigen CD2 (T11); Also: M14362 | + |
| U12404 | Csa-19 | − |
| M74091 | Cyclin C | + |
| U73737 | HMSH6 | − |
| U62801 | protease M | + |
| X97267 | LPAP; Also: X81422 | + |
| D87845 | platelet-activating factor acetylhydrolase 2 | + |
| L42243 | IFNAR2 (interferon receptor) | + |
| M64595 | Small G prot (Gx) | + |
| L25286 | Alpha-1 type XV collagen; Also: L25285 | − |
| HG3513-HT3707 | Myosin, Heavy Polypeptide, Light Meromyosin | + |
| Y09836 | unknown prot | − |
| X97335 | kinase A anchor prot | − |
| HG1155-HT4822 | Colony-Stimulating Factor 1 Macrophage (CSF1) | + |
| HG2007-HT2056 | Proto-Oncogene Sno; Also: HG2007-HT5112 | + |
| M61764 | gamma-tubulin | − |
| AF001548 | myosin heavy chain from chromosome 16 | − |
| L07044 | Ca-Calmodulin-dependent prot kinase CAMK | − |
| D31889 | 26S proteasome subunit S5 basic/KIAA0072 | − |
| Z49989 | smoothelin | − |
| M34344 | platelet glycoprot IIb (GPIIb) | + |
| L04490 | NADH-ubiquinone oxidoreductase subunit (NDUFA9) | − |
| X52896 | dermal fibroblast elastin; Also: HG2994-HT4851 | + |
| J05272 | IMP dehydrogenase type 1 | + |
| L26339 | autoantigen | − |
| L42374 | PP2A B56-beta; Also: Z69028 | + |
| U20350 | G prot-coupled receptor V28 | + |
| X84194 | acylphosphatase erythrocyte (CT) isoenzyme | + |
| X61587 | rhoG GTPase | + |
| U02566 | receptor tyrosine kinase TIF; Also: U18934 | − |
| X52979 | SmB prot from small nuclear ribonucleoprots; Also: X17567 | − |
| U19523 | GTP cyclohydrolase 1 | + |
| X06825 | skeletal beta-tropomyosin | − |
| J04611 | lupus p70 (Ku) autoantigen prot | − |
| U43527 | malignant melanoma metastasis-suppressor (KiSS-1) | − |
| X98743 | RNA helicase (Myc-regulated dead box prot) | + |
| L10844 | cellular growth-regulating prot | − |
| U43203 | Thyroid transcription factor 1 (TTF-1); Also: U33749 | + |
| U50327 | prot kinase C substrate 80K-H (PRKCSH); Also: J03075 | + |
| L41690 | TNF receptor-1 associated prot (TRADD) | + |
| M99701 | pp21 | − |
| V00594 | metallothionein 2A (MT2A); Also: J00271 | − |
| U40992 | heat shock prot hsp40 homolog | + |
| M33374 | cell adhesion prot (SQM1) | − |
| HG831-HT831 | Potassium Channel | + |
| HG658-HT658 | Major Histocompatibility Complex, Class I, C X58536) | + |
| U68019 | mad prot homolog (hMAD-3) | − |
| D16581 | 8-oxo-dGTPase | + |
| L14813 | carboxyl ester lipase like prot (CELL) | + |
| X13973 | ribonuclease/angiogenin inhibitor (RAI) | − |
| L10377 | (clone CTG-B37) sequence; Also: D38529, U23851, D31840 | − |
| Z68129 | NAD(H)-specific isocitrate dehydrogenase gamma-subunit | − |
| L19783 | GPI-H | − |
| HG4018-HT4288 | Opioid-Binding Cell Adhesion Molecule | + |
| L10910 | splicing factor (CC13) | − |
| U30246 | bumetanide-sensitive Na-K-CI cotransporter (NKCC1) | − |
| HG4683-HT5108 | TNF Receptor 2 Associated prot Trap3; Also: U12597 | − |
| M31013 | nonmuscle myosin heavy chain (NMHC) | − |
| U94832 | KH type splicing regulatory prot KSRP | − |
| S69272 | 38 kda intracellular serine protase inhibitor; Also: Z22658 | − |
| U76010 | putative zinc transporter ZnT-3 (ZnT-3) | + |
| L39064 | IL-9 receptor | − |
| M73547 | polyposis locus (DP1 ) | − |
| HG2668-HT2764 | Bradykinin Receptor | + |
| Z50115 | Thimet oligopeptidase (metalloproteinase); Also: U29366 | − |
| M21259 | Alu repeats in the region to the Snrp E | − |
| X95073 | translin associated prot X | + |
| V01516 | cytoskeletal keratin (type II) from foreskin | + |
| X70940 | elongation factor 1 alpha-2; Also: L10340 | − |
| D16350 | SA | + |
| AF007551 | Bet1p homolog (hbet1) | + |
| D50692 | c-myc binding prot | + |
| U34044 | selenium donor prot (selD) | − |
| U48959 | myosin light chain kinase (MLCK) | − |
| U54644 | tub homolog; Also: U82467 | − |
| U59057 | beta-A4 crystallin (CRYBA4) | − |
| U66559 | anaplastic lymphoma kinase receptor | − |
| L11701 | phospholipase D; Also: L11702 | + |
| L39874 | deoxycytidylate deaminase | + |
| U33052 | lipid-activated, prot kinase PRK2; Also: S75548 | + |
| U18671 | Stat2 | + |
| M59371 | prot tyrosine kinase | + |
| Y10615 | CYRN2/Y10615 | + |
| HG4116-HT4386 | Olfactory Receptor Or17-219; Also: HG4108-HT4378 | − |
| U15460 | bZip prot B-ATF | + |
| X62822 | beta-galactoside alpha-26-sialyltransferase | + |
| U22233 | methylthioadenosine phosphorylase (MTAP) | + |
| X59710 | CAAT-box DNA binding prot subunit B (NF-YB) | + |
| D87077 | hypothetical protein/KIAA0240 | + |
| U31875 | Hep27 prot. | − |
| D29956 | ubiquitin specific protease 8/KIAA0055 | + |
| U30998 | (nmd)/U30998 | − |
| U23852 | X13529, X05027 and others | + |
| Y07847 | RRP22 prot | + |
| M93107 | heart (R)-3-hydroxybutyrate dehydrogenase end | + |
| Z22551 | kinectin | − |
| HG1800-HT1823 | Ribosomal prot S20 | + |
| D13635 | ubiquitin-protein isopeptide ligase E3/KIAA0010 | + |
| M19961 | cytochrome c oxidase subunit Vb (coxVb) | − |
| X82850 | thyroid transcript factor 1; Also: U43203, U33749 | − |
| S82447 | GCN5-like 1 = GCN5 homolog/Also: D64007 | − |
| M16364 | creatine kinase-B; Also: X15334_rna1 | − |
| M10612 | apolipoprot C-II | + |
| X13293 | v-myb myeloblastosis viral homolog-like 2 (MYBL2) | − |
| U47101 | NifU-like prot (hNifU) | − |
| U65579 | mitoch NADH dehydrogenase-ubiquinone Fe-S prot 8 23 kDa | − |
| D10995 | serotonin 1B receptor | + |
| U51586 | siah binding prot 1 (SiahBP1) | − |
| D50477 | membrane-type matrix metalloproteinase 3; Also: D83646 | + |
| S45630 | alpha B-crystallin = Glioma Rosenthal fiber component | − |
| J02923 | 65-kilodalton phosphoprot (p65) | + |
| S85655 | prohibitin (PHB) | − |
| X14046 | leukocyte antigen CD37 | + |
| U57627 | fetal brain oculocerebrorenal syndrome (OCRL1) | − |
| M26004 | CR2/CD21/C3d/Epstein-Barr virus receptor; Also: J03565 | + |
| HG1980-HT2023 | Tubulin, Beta 2 | − |
| U07231 | G-rich sequence factor-1 (GRSF-1) | − |
| M86528 | neurotrophin-4 (NT-4) | + |
| D28476 | thyroid hormone receptor interactor 12/KIAA0045 | + |
| U73167 | H_LUCA146 | − |
| U37146 | silencing mediator of retinoid and thyroid hormone (SMRT) | − |
| D64053 | prot-tyrosine phosphatase | − |
| D14889 | small GTP-binding protein S10 (RAB33A) | + |
| X52638 | 6-phosphofructo-2-kinase/fructose-26-bisphosphatase | + |
| U96629 | Hereditary multiple exostosis | − |
| M85247 | dopamine D1A receptor/M85247 | − |
| Y08263 | AAD14 prot | + |
| X57766 | stromelysin-3 | − |
| D13644 | tre oncogene homolog/KIAA0019 | + |
| U77665 | RNaseP prot p30 (RPP30) | − |
| X74104 | TRAP beta subunit | − |
| D78132 | Ras homolog enriched in brain (RHEB); Also: Z29677 | − |
| U79271 | clones 23920 and 23921 sequence | − |
| X52851 | peptidylprolyl isomerase from cyclophilin (EC 5.2.1.8) | − |
| U19977 | preprocarboxypeptidase A2 (proCPA2) | + |
| X04470 | antileukoprotease (ALP) from cervix uterus; Also: X04503 | + |
| D30742 | calmodulin-dependent prot kinase IV | + |
| D26070 | type 1 inositol 145-trisphosphate receptor | − |
| U03399 | T-complex prot 10A (TCP10A) | + |
| X03068 | HLA-D class II antigen DQw1.1 beta chain | + |
| X77548 | RFG | + |
| U07418 | DNA mismatch repair (hmlh1) | − |
| U73799 | dynactin/U73799 | + |
| X54667 | cystatin S; Also: S51222, M19169 | − |
| L40397 | (clone S31i125) | − |
| M23533 | alpha 2 adrenergic receptor | + |
| U11732 | ets-like (tel) | + |
| D17400 | 6-pyruvoyl-tetrahydropterin synthase | − |
| D11086 | interleukin 2 receptor gamma | + |
| M24248 | MLC-1V-Sb | + |
| M20867 | glutamate dehydrogenase (GDH); Also: HG4200-HT4470 | + |
| L38820 | HMC class I antigen-like glycoprot (CD1D) | + |
| J04132 | T cell receptor zeta-chain | + |
| M31932 | lgG low affinity Fc fragment receptor (FcRlla) | + |
| X14008 | lysozyme (EC 3.2.1.17) | + |
| U63295 | Seven in absentia homolog | − |
| U06631 | (H326) | − |
| X80822 | ORF | + |
| L01406 | growth hormone-releasing hormone receptor | + |
| M68895 | alcohol dehydrogenase 6 | − |
| X60036 | mitochondrial phosphate carrier prot | − |
| M25079 | Sickle cell beta-globin; Also: U01317_6, HG1428-HT1428 | + |
| X69978 | XP-G factor | − |
| L08835 | DM kinase (myotonic dystrophy kinase)/HG2486-HT2582 | − |
| M34668 | prot tyrosine phosphatase (PTPase-alpha) | − |
| U61145 | enhancer of zeste homolog 2 (EZH2) | − |
| U62966 | Na+/nucleoside cotransporter (hCNT1a) | − |
| L19401 | myosin I homolog (MYH12) | + |
| U85767 | myeloid progenitor inhibitory factor-1 (MPIF1) | + |
| U94586 | NADH: ubiquinone oxidoreductase MLRQ subunit | − |
| L13972 | beta-galactoside alpha-23-sialyltransferase (SIAT4A) | − |
| X57152 | casein kinase II subunit beta (EC 2.7.1.37); Also: M30448 | − |
| U50950 | infant brain unknown product | − |
| X15882 | collagen VI alpha-2 C-terminal globular domain | − |
| U09850 | zinc finger prot (ZNF143) | + |
| X76013 | QRSHs glutaminyl-tRNA synthetase | − |
| X05855 | histone H3.3; Also: M11353 | + |
| X16663 | HS1 heamatopoietic lineage cell specific prot | + |
| M22760 | mitochondrial cytochrome c oxidase Va subunit | − |
| HG3412-HT3593 | Blue Cone Photoreceptor Pigment; Also: M13299 | + |
| U47292 | spasmolytic polypeptide (SP) | + |
| X13546 | put. HMG-17 non-histone prot | − |
| M18000 | ribosomal prot S17 | − |
| Z68193 | Opsin 1 (OPN1LW) | + |
| L49173 | OCP2/L49173 | + |
| X85750 | monocyte to macrophage differentiation-associated prot | + |
| X84373 | nuclear factor RIP140 | − |
| D87743 | Sodium/hydrogen exchanger 6/KIAA0267 | + |
| Z12173 | GNS glucosamine-6-sulphatase | + |
| U60115 | skeletal muscle LIM-prot SLIM1 | − |
| HG4263-HT4533 | Nkr-P1a prot | + |
| U30245 | myelomonocytic specific prot (MNDA)/U30245 | + |
| X72841 | IEF 7442 | − |
| M64571 | microtubule-associated prot 4 | − |
| M23294 | beta-hexosaminidase beta-subunit (HEXB) | − |
| J03133 | TRANSCRIPTION FACTORS SP1 | + |
| L40636 | (clone FBK III 16) prot tyrosine kinase (NET PTK) | − |
| X54870 | NKG2-D | + |
| M26167 | platelet factor 4 varation 1 (PF4var1) | + |
| U59058 | beta-A3/A1 crystallin (CYRBA3/A1); Also: M14306 | + |
| U89336 | Notch 4 | + |
| Z11559 | iron regulatory factor | − |
| HG4322-HT4592 | Tubulin, Beta | − |
| X06256 | fibronectin receptor alpha subunit | − |
| U13395 | oxidoreductase (HHCMA56) | + |
| D79990 | Ras association domain family 2/KIAA0168 | + |
| U90916 | clone 23815 sequence, IFN-inducible | + |
| AB002318 | talin homolog/KIAA0320 | + |
| X55448 | G6PD glucose-6-phosphate dehydrogenase; Also: M65234 | + |
| D89016 | Neuroblastoma | + |
| U13737 | cysteine protease CPP32 isom alpha | − |
| U11690 | faciogenital dysplasia (FGD1) | − |
| X02176 | complement component C9; Also: K02766 | − |
| HG2075-HT2137 | Camp-Responsive Element Modulator; D14825, S68134 | + |
| U39400 | NOF1 | − |
| U68723 | checkpoint suppressor 1 | − |
| L11373 | protocadherin 43 | − |
| D25216 | hypothetical protein/KIAA0014 | − |
| U13695 | homolog of yeast mutL (hPMS1) | + |
| U67611 | Mouse transaldolase/U67611 | + |
| X84746 | Histo-blood group AB0 1 | − |
| AF012270 | peropsin (Rrh) | − |
| J03925 | Mac-1 encoding complement receptor type 3 CD11b | + |
| Z74792 | CCAAT transcription binding factor subunit gamma/S74703 | − |
| U79300 | clone 23629 sequence | + |
| M11313 | alpha-2-macroglobulin; Also: M36501 | − |
| M30838 | pulmonary surfactant apoprot (PSAP) | + |
| HG1595-HT4788 | Heterogeneous Nuclear Ribonucleoprot I/X66975 | + |
| M27396 | asparagine synthetase; Also: M15798 | − |
| L48513 | paraoxonase 2 (PON2) | − |
| U50939 | amyloid precursor prot-binding prot 1 | − |
| L41607 | beta-16-N-acetylglucosaminyltransferase (IGnT) | + |
| D00654 | Enteric smooth muscle gamma-actin | − |
| Y12711 | putative progesterone binding prot | + |
| D86963 | dishevelled 3/KIAA0208 | + |
| L37043 | Casein kinase I epsilon | − |
| U60415 | bHLH-PAS prot Jap3 | + |
| L76937 | unnamed prot product from Werner syndrome | + |
| X98258 | M-phase phosphoprot mpp9 | + |
| M14636 | liver glycogen phosphorylase | + |
| V00571 | Prepro form of corticotropin releasing factor | − |
| D55696 | cysteine protease | − |
| U79272 | clone 23720 sequence | + |
| M27691 | transactivator prot (CREB) | + |
| U52111 | Plexin related prot | − |
| U83239 | CC chemokine STCP-1; Also: U83171 | + |
| X61072 | T cell receptor, clone IGRA17 | + |
| S71824 | N-CAM | − |
| M27093 | alpha-keto acid dehydrogenase transacylase subunit E2b | + |
| U19878 | transmembrane prot | + |
| HG2649-HT2745 | Serine/Threonine prot Kinase Cdk3; Also: X66357 | − |
| D89289 | N-Acetyl-beta-D-glucosaminide | + |
| L33243 | polycystic kidney disease 1 prot (PKD1) | − |

TABLE 13
Highest discriminatory genes for MS vs. MS on Avonex
(i.e., genes modified by Avonex).

| | | Up (+) or |
|---|---|---|
| Probe sets | Gene Descriptions | down (−) |
| U60115 | Four and a half LIM skeletal muscle SLIM1 | + |
| | (FHL1) | |
| X80878 | R kappa B (NFRKB) | + |
| AF001548 | 815A9.1 myosin heavy chain (MYH11) | − |
| K03008 | Gamma G2 psi from gamma crystalline | − |
| | (CRYGEP1) | |
| Z19585 | thrombospondin-4 (THBS4) | + |
| Z97054 | PAC contains KIAA0178 similar to SMC1 and | + |
| | URE-B1 | |

TABLE 14
Intermediate discriminatory genes for MS vs. MS on
Avonex (i.e., genes modified by Avonex)

| | | |
|---|---|---|
| M94345 | macrophage capping protein (CAPG) | − |
| M21665 | beta-myosin heavy chain (MYH7); Also: X52889 | + |
| M30448 | X56597, M59849 and others | + |
| X16416 | c-abl encoding p150 prot | + |
| Z75330 | nuclear protein stromal antigen SA-1 (STAG1) | + |
| X79865 | Mrp17/Mitochondrial ribosomal L12 (MRPL12) | − |
| X60483 | H4/D histone | + |
| M96759 | rod outer segment membrane prot 1 (ROM1) | − |
| M36284 | glycophorin C (GYPC); Also: X12496 | + |
| U88666 | SFRS protein kinase 2 (SRPK2) | + |
| D86973 | yeast translational activator Gcn1 homolog (GCN1L1) | + |
| U82130 | tumor susceptibility protein (TSG101) | + |
| S80050 | a-6-D-mannoside b-16-N-acetylglucosaminyltransferase V | + |

TABLE 15
Least discriminatory genes for MS vs. MS on
Avonex (i.e., genes modified by Avonex)

| | | |
|---|---|---|
| U90909 | clone 23722 sequence | + |
| M22638 | Lymphoblastic leukemia-derived sequence 1 (LYL1) | − |
| U07807 | metallothionein IV (MTIV) | + |
| HG2825- | Ret Transforming | + |
| HT2949 | | |
| X71428 | Fus | + |
| S76617 | protein tyrosine kinase (BLK) | + |
| HG1428- | Globin, Beta (HBB); Also: U01317_6 | + |
| HT1428 | | |
| S82597 | UDP-GaINAc: N-acetylgalactosaminyltransferase | + |
| | (GALNT1) | |
| U26710 | cbl-b (CBLB) | − |
| X62078 | GM2 activator protein (GM2A) | − |
| X76057 | PMI1 phosphomannose isomerase | + |
| HG4677- | Oncogene Ret/Ptc2, Fusion Activated/M57464 | − |
| HT5102 | | |
| L10102 | sex determining region Y (SRY) | − |
| M93143 | plasminogen-like protein (PLGL) | + |
| U79261 | clone 23959 (MAPK8IP2); Also: U62317 | + |
| X59373 | HOX4D homeobox protein | + |
| X83218 | ATP synthase | + |
| M80563 | Calcium-binding CAPL protein | − |
| M34458 | lamin B1 (LMNB1); Also: M34458, L37747 | − |
| L32866 | effector cell protease receptor-1 (EPR-1) | + |
| U10117 | endothelial-monocyte activating polypeptide II | − |
| U25138 | MaxiK potassium channel beta subunit (KCNMB1) | − |
| U78095 | Placental bikunin (AMBP) | + |
| D38550 | E2F3 transcription factor/KIAA0075 | + |
| X64878 | oxytocin receptor (OXTR) | − |
| U70323 | Ataxin-2 (SCA2) | − |
| D21878 | Bone marrow stromal cell antigen 1 (BST1) | − |
| X90530 | ragB protein | + |
| M31627 | X box binding prot-1 (XBP-1) | + |
| Y10207 | CD171 protein | + |
| U66559 | anaplastic lymphoma kinase receptor (ALK) | − |
| L12723 | heat shock prot 70 (hsp70) protein 4 (HSPA4) | − |
| D87685 | TFIIS-like PHD finger protein 3/ | + |
| | KIAA0244 | |
| J04982 | adenine nucleotide translocator ANT1 (SLC25A4) | + |
| X65867 | adenylosuccinate lyase (ADSL) | + |
| U83598 | death domain receptor 3 soluble form DDR3 | + |
| | (TNFRSF25) | |
| M93426 | prot tyrosine phosphatase zeta-polypeptide (PTPRZ1) | + |
| U01147 | guanine nucleotide regulatory protein (ABR) | − |
| U78027 | L44L (L44-like ribosomal protein) | − |
| D50911 | hypothetical protein/KIAA0121 | + |
| S77361 | transcript ch132/S77361 | − |
| X75252 | phosphatidylethanolamine binding protein | + |
| L09235 | vacuolar ATPase isom VA68 (ATP6V1A1) | − |
| X00274 | HLA-DR alpha heavy chain a class II antigen | − |
| D11094 | Proteasomal ATPase MSS1 (PSMC2) | − |
| M19483 | ATP synthase beta subunit | − |
| U88964 | HEM45 | − |
| D87450 | parallel sister chromatids drosophila protein-like/ | + |
| | KIAA0261 | |
| U65406 | KCNJ1 (from potassium channel ROM-K1-6) | − |
| X83492 | Fas/APO1 (TNFRSF6) | + |
| U17077 | BENE protein | + |
| U49352 | liver 24-dienoyl-CoA reductase 1 (DECR1) | − |
| Z25535 | nuclear pore complex protein hnup153 | + |
| M21119 | lysozyme (LYZ); Also: X14008_rna1, J03801, M19045 | − |
| M87284 | 69 kDa 2′ oligoadenylate synthetase (P69 2-5A | − |
| | synthetase) | |
| L15189 | mortalin-2/L11066 | + |
| U67784 | orphan G protein-coupled receptor (RDC1) | − |
| D87076 | Br140/KIAA0239 | + |
| U10323 | nuclear factor NF45 | + |
| M34455 | IFN-gamma-inducible indoleamine 23-dioxygenase | − |
| | (INDO) | |
| M11147 | ferritin L chain | − |
| L36033 | pre-B cell stimulating factor homolog SDF1b (CXCL12) | + |
| Y07604 | nucleoside-diphosphate kinase | + |
| M27543 | guanine nucleotide-binding prot (Gi) alpha subunit | − |
| | (GNAI3) | |
| S54005 | thymosin beta-10; Also: M92383 | − |
| AC002477 | PAC clone DJ327A19 from Xq25-q26, AC002477/ | + |
| | X98253 | |
| D80004 | Hypothetical protein/KIAA0182 | + |
| U34044 | selenium donor prot selenophosphate synthetase (SPS) | + |
| M30607 | zinc finger prot Y-linked (ZFY); Also: L10393 | − |
| D38521 | rat TEMO homolog/KIAA0077 | + |
| X66401 | LMP2 from TAP1, TAP2, LMP2, LMP7 and DOB | − |
| U00001 | Cell division cycle 27 (CDC27)/S78234 | − |
| D80005 | hypothetical protein/KIAA0183 | + |
| U56833 | VHL binding protein-1 (VBP-1) | + |
| M27396 | asparagine synthetase (ASNS); Also: M15798 | + |
| X56253 | MPR46 46 kd mannose 6-phosphate receptor (M6PR) | − |
| X77753 | TROP-2 (TACSTD2) | + |
| J04040 | Glucagon (GCG) | − |
| L14813 | carboxyl ester lipase like prot (CELL) | − |
| AF003743 | delayed rectifier potassium channel (KVLQT1-Iso5) | − |
| U52154 | G prot-coupled inwardly rectifying potassium channel | + |
| | Kir34 | |
| U56998 | putative serine/threonine protein kinase PRK | + |
| X99209 | arginine methyltransferase | + |
| Z30643 | chloride channel (CLCNKA) | − |
| D14662 | anti-oxidant protein peroxiredoxin 6 (PRDX6)/ | + |
| | KIAA0106 | |
| U32576 | apolipoprot apoC-IV (APOC4) | − |
| D89377 | Msh homeobox homolog 2 (MSX2) | − |
| L22005 | ubiquitin conjugating enzyme | + |
| U03100 | alpha2(E)-catenin | + |
| U05237 | fetal Alz-50-reactive clone 1 (FAC1) | + |
| L05425 | Autoantigen | − |
| J03909 | gamma-interferon-inducible protein (IP30) | − |
| M22976 | cytochrome b5 | − |
| X97324 | adipophilin (ADFP) | + |
| HG3369- | Potassium Channel Voltage-Gated lsk-Related | − |
| HT3546 | | |
| D89859 | zinc finger 5 protein (ZNF5) | + |
| X66363 | PCTAIRE-1 serine/threonine prot kinase 1 (PCTK1) | − |
| D90084 | pyruvate dehydrogenase alpha subunit 1 (PDHA1) | + |
| D13315 | lactoyl glutathione lyase, glyoxalase 1 (GLO1) | + |
| J04101 | NAD(P)H: menadione oxidoreductase; Also: M81600 | − |
| U89012 | dentin matrix acidic phosphoprotein 1 (DMP1) | − |
| D42041 | alpha glucosidase 2 (G2AN)/KIAA0088 | + |
| D31890 | lysyl-tRNA synthetase (KARS)/KIAA0070 | + |
| U53830 | interferon regulatory factor 7A (IRF7) | − |
| U15085 | HLA-DMB | − |
| L20941 | ferritin heavy chain (FTH1) | − |
| X13293 | v-myb myeloblastosis viral homolog-like 2 (MYBL2) | − |
| U07158 | Syntaxin | − |
| M77144 | 3-b-hydroxysteroid dehydrogenase/5delta 4delta | + |
| | isomerase | |
| X55079 | GAA from lysosomal alpha-glucosidase 1 | + |
| U79254 | clone 23693 sequence | + |
| X98507 | myosin-l beta | + |
| U44975 | DNA-binding protein CPBP (CPBP) | − |
| U49278 | putative DNA-binding protein | − |
| D85376 | DNA thyrotropin-releasing hormone receptor (TRHR) | + |
| X85785 | DARC, Duffy blood group (FY) | + |
| M19507 | Myeloperoxidase (MPO) | − |
| M26311 | M21064 and others; Calgranulin B (S100A9) | − |
| M26041 | MHC class II DQ alpha 1 (HLADQA1); Also: M33906 | − |
| M95929 | Paired mesoderm homeobox protein 1 PHOX1 (PMX1) | − |
| X15414 | aldose reductase (EC 1112) | + |
| L05188 | small proline-rich protein 2 (SPRR2B) | + |
| L34219 | retinaldehyde-binding protein (CRALBP) | − |
| X74262 | RbAp48 encoding retinoblastoma binding protein | + |
| M88461 | neuropeptide Y peptide YY receptor (NPY1R); L07615 | − |
| M90696 | cathepsin S (CTSS) | − |
| X54150 | Fc receptor (FCAR) | − |
| M23254 | Ca2-activated neutral protease large subunit (CANP) | − |
| M27891 | cystatin C (CST3) | − |
| L20591 | annexin III (ANX3) alternative | − |
| M57731 | gro-beta; chemokine (C-X-C) motif ligand 2 (CXCL2) | − |
| U88902 | endogenous retroviral integrase H and putative env prot | + |
| M59941 | GM-CSF receptor beta chain (CSF2RB) | + |
| M80629 | cdc2-like protein kinase 5 (CDC2L5) | + |
| X72790 | endogenous retrovirus | + |
| D64015 | T-cluster binding protein | + |
| D17516 | PACAP receptor (ADCYAP1R1) | − |
| Z26491 | catechol O-methyltransferase (COMT); Also: M65213 | + |
| U91931 | AP-3 complex beta3A subunit | − |
| X53002 | integrin beta-5 subunit; Also: J05633 | + |
| M12174 | ras-related rho (clone 6) | − |
| X04500 | prointerleukin 1 beta (IL1B) | − |
| D15057 | Defender against cell death 1 (DAD1) | + |
| D50912 | RNA-binding motif protein-10 (RBM10)/KIAA0122 | + |
| M34338 | spermidine synthase (SRM); Also: M64231_rna1 | + |

TABLE 16
Preferred Genes List Number 1: Genes that discriminate blood
samples of MS (on no treatment) when compared to ALS and Healthy
donors, regardless of heterogeneity of the MS sample population.

| | | Up (+) or |
|---|---|---|
| Probe set | Gene description | down (−) |
| D30037 | phosphatidylinositol transfer protein (PITPN) | + |
| D29675 | Inducible nitric oxide synthase (iNOS) | + |
| Z25884 | CIC-1 muscle chloride channel protein (CLCN1) | + |
| U78095 | Placental bikunin (AMBP) | + |
| U14187 | receptor tyrosine kinase ligand LERK-3/ | + |
| | Ephrin-A3 | |
| L34357 | GATA4 | + |
| U18271 | thymopoietin (TMPO) | + |
| HG2415- | transcription factor E2F2 | + |
| HT2511 | | |

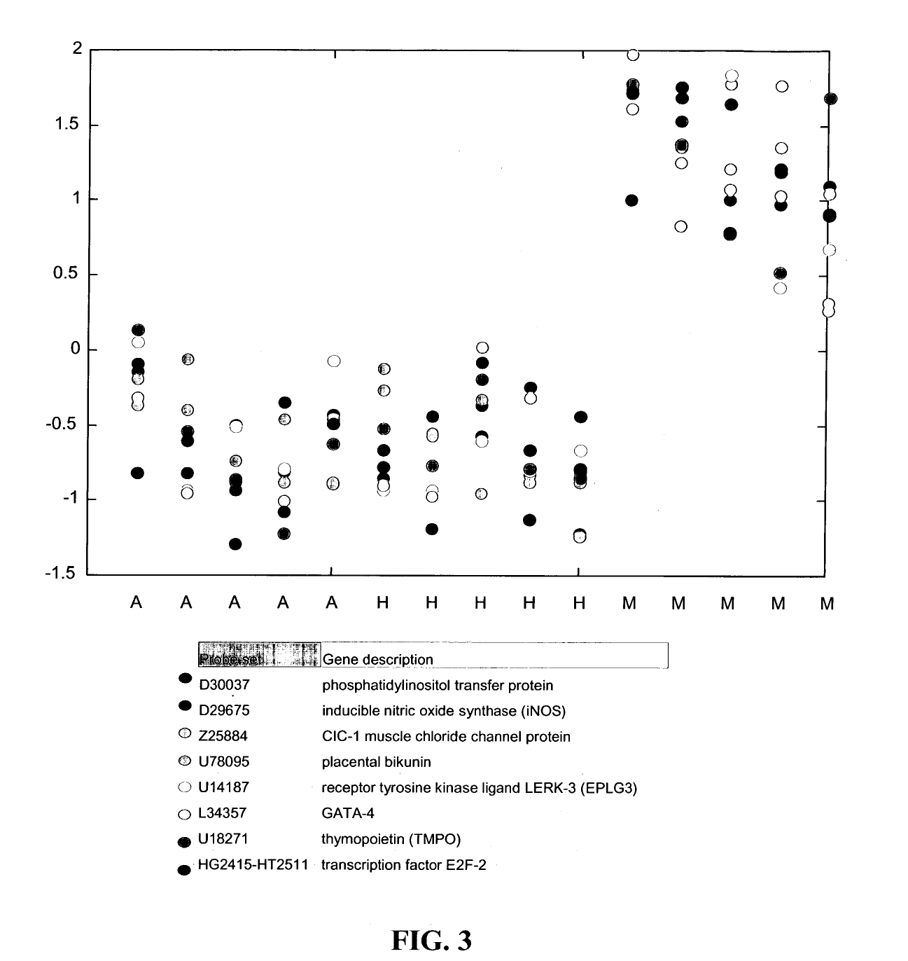

Tables 1-15 include comparisons of a homogeneous MS patient population (i.e., exclusively the relapsing-remitting type of MS, on Avonex or not, 5 samples each) with ALS (5 samples) or healthy donors (5 samples). To establish genes that were consistently discriminatory for MS regardless of patient sample heterogeneity, the inventors added a blood sample from an untreated patient who presented with tumefactive, tumor-like demyelinative lesions by MRI imaging (i.e., an extreme clinical variant of the disease refered to as “tumefactive” MS, which was confirmed by brain biopsy) and a patient with 6 years of symptoms of multiple sclerosis (confirmed by MRI imaging), now entering the secondary progressive phase of the disease and who had not previously received any specific treatment for her condition (total number of patients with MS=7). The inventors also added 2 blood samples from healthy donors (total=7) and 3 blood samples from ALS patients (total=8). They then performed a comparison of MS vs. ALS and healthy donors, and established the highest discriminatory genes that were shared with Tables 7-9 (which include only 5 samples in each group, including the homogeneous MS population). Among these shared discriminatory genes, they chose 8 top genes that are highly discriminatory in analyzing blood samples from untreated MS (regardless of heterogeneity of MS) when compared to healthy donors and/or other neurodegenerative (not primarily autoimmune) diseases (e.g., ALS) (Table 16). For purposes of uniformity, these 8 genes are shown in a comparison using 5 patients in each group (see FIG. 3).

TABLE 17
Preferred Genes List Number 2: Independent of Treatment or Controls

| | Probe Set | Altered |
|---|---|---|
| Gene Description | Identification | In* |
| S-adenosylmethionine synthetase | X68836 | 1, 2, 3 |
| (MAT2A) | | |
| Carcinoembryonic antigen | HG3175-HT3352 | 1, 2, 3 |
| Ret transforming gene | HG2825-HT2949 | 1, 2, 3 |
| G protein-linked receptor (clone | L42324 | 1, 2 |
| GPCR W) | | |
| GTP-binding protein RALB | M35416 | 1, 2 |
| Tyrosine kinase Syk | L28824 | 1, 2 |
| T cell leukemia LERK-2 (Ephrin-B1) | U09303 | 1, 2 |
| Tyrosine kinase (ELK1) oncogene | M25269 | 1, 2 |
| Transcription factor SL1⁺ | L39059 | 1, 3 |
| Phospholipase c (PLCB3)⁺ | Z16411 | 1, 3 |
| Gastricsin (progastricsin) | U75272 | 2, 3 |
| D13S824E locus⁺ | U47635 | 2,3 |
| *1: Avonex-treated heterogeneous MS and untreated heterogeneous MS vs. ALS and healthy | | |
| #donors; 2: untreated heterogeneous MS vs. ALS and healthy donors; 3: untreated heterogeneous | | |
| #MS vs. healthy donors | | |

⁺downregulated genes

The second group of preferred genes, shown in Table 17, was generated by multiple group comparisons using the inventors' statistical method, with the goal being to identify a heterogeneous population of MS patients, regardless of treatment with interferon β1a, and regardless of control sample origin (healthy individuals or subjects with other neurodegenerative diseases used as controls). Heterogeneity is used in this context to refer to a population of patients with different phenotypic forms of MS (i.e., besides the classical relapsing-remitting clinical form with typical ovoid, pericallosal or periventricular lesions by MR imaging and little disability in its early stages). Such patients may have the secondary progressive form and may have accumulated more clinical disability, may not have any brain lesions but only optic nerve or spinal cord demyelinating lesions, may have brain atrophy by MR imaging, may have brain tumor-mimicking MS lesions (i.e., tumefactive MS), and may or may not be on treatment with interferon β1a. MS patients in the group ranged in age from 18 to 44 years (within the normal distribution for MS).

To generate the list in Table 17, the inventors repeated their analysis by further increasing the number of RNA samples in each group (by adding RNA samples from a new set of blood donors to the original homogeneous MS groups), which increased the stringency of selection of heterogeneous MS-associated genes. They compared 8 untreated MS samples (heterogeneous types of MS), 9 Avonex-treated heterogeneous MS samples, 7 healthy donor samples, and 8 ALS samples (total: 32 samples). The following comparisons among grouped classes were performed: (1) Avonex-treated heterogeneous MS and untreated heterogeneous MS vs. ALS and healthy donors (two groups total), which generated 25 discriminatory genes; (2) untreated heterogeneous MS vs. ALS and healthy donors (two groups total), which generated 27 discriminatory genes; (3) untreated heterogeneous MS vs. healthy donors (two groups total), which generated 16 discriminatory genes. They then selected genes that were shared across these highest discriminatory lists. Unless specifically noted as downregulated, all the genes in Table 17 are upregulated in MS. The genes already present in preferred list 1 are omitted.

While both the lists in Tables 16 and 17 provide preferred targets, other equally valuable lists, using different criteria (distinct patient populations, different types of MS, different age of onset, etc.) may be constructed.

1. Discussion of Gene and Pathways Involved in MS

The following paragraphs represent a discussion of important pathways implicated in MS by the inventors' studies, and several key member molecules, found to be altered in MS peripheral blood by the inventors (see Tables above). Although the involvement of some of these genes might be inferred by in vitro or post-mortem experiments, there is very little in vivo evidence for most of these genes in MS. The present invention, which examines “immediately-ex vivo” material, is unique in approximating the in vivo setting, and provides far more conclusive evidence of their involvement.

Phosphatidylinositol transfer protein is upregulated in MS peripheral blood, and this protein serves as a lipid transport molecule (for membrane sphingolipids, for instance) and works at the interface of phospholipase c signaling and in membrane trafficking (including exocytosis and vesicle biogenesis). LERK-3 (EPLG3) is an ephrin-related receptor tyrosine kinase ligand that has been studied with relation to the nervous system but not to the immune system and has not been previously implicated in MS.

Several studies have implicated the nitric oxide (NO) pathway as a contributing factor in the pathogenesis of MS and of several other neurodegenerative or neuroinflammatory disorders. NO synthase leads to NO production, and NO reacts with superoxide anion to produce peroxynitrite (ONOO′). The stable end products of peroxynitrite (i.e., nitrite (NO2′) and nitrate (NO3′)) are commonly measured as an indirect evidence of NO-related oxidative damage. (Sarchielli et al., 1997) described higher production of nitrites in the supernatant of non-LPS-stimulated peripheral blood mononuclear cells of MS patients but only in patients with active lesions, or in patients undergoing disease relapses (also known as attacks or exacerbations). Others (Giovannoni et al. 1997) demonstrated elevated nitrite and nitrate in serum of patients with MS and in those with other neuro-inflammatory diseases, regardless of their level of disability. De Groot et al., 1997, has shown that in active MS lesions, there is strong iNOS immunoreactivity in perivascular and intralesional macrophages, indicating a possible role for this pathway in triggering cytotoxicity of oligodendrocytes and demyelination. Bagasra et al. (1995) also described high levels of iNOS in macrophages from MS postmortem brains. Bo et al., 1994, found iNOS mRNA elevation in demyelinating lesions of MS. Svenningsson et al. (1999), demonstrated increased nitrite levels in the cerebrospinal fluid (CSF) of MS patients with an active course. Yuceyar et al. (2001), found elevation of nitrate and nitrite in both serum and CSF of MS patients, independently of disease type, presence of exacerbation or degree of disability. Yamashita et al., (1997), demonstrated elevated CSF nitrate and nitrite at the most extreme stages of MS exacerbations. Liu et al. (2001), found higher expression of iNOS mRNA and protein in inflammatory cells and endothelial cells within early MS lesions, and in astrocytes at the edge of the lesion in chronic plaques. Lopez-Moratalla et al. (1997) demonstrated by immunocytochemistry and flow cytometry that iNOS was augmented in a subset (CD16+) of macrophages from peripheral blood. Importantly, Ding et al. (1998) has shown that using antisense oligodeoxynucleotides to specifically block the expression of iNOS in mice leads to resistance to the induction of experimental autoimmune encephalomyelitis (i.e., a disease induced in mice that represents the best known animal model of MS). Thus, DNA microarray experiments of peripheral blood detect mRNA changes that point towards potential targets of treatment, as exemplified by identification of iNOS elevation.

Bikunin represents the inhibitory light chain of the inter-α-trypsin inhibitor protein. It is a protease inhibitor, known to be elevated in the urine of patients with inflammatory diseases, such as psoriasis (Streit et al, 1995) and is considered an acute phase protein. However, there are no reports studying the role of bikunin in MS. The inventors reveal elevation of bikunin mRNA in MS suggesting that it is indeed a marker of inflammatory activity for this disease.

GATA-4 is a transcription factor that binds to genomic DNA containing GATA (genetic code) sequences. By itself, GATA-4 seems to be able to relax (decondense) compacted chromatin, suggesting a potential role in facilitating gene activation. It has been implicated in selectively upregulating interleukin 5, and may contribute to immune cell differentiation. Its role in immune cells to date is less understood than that of another family member, GATA-3, which is implicated in maintaining a Th2 (pro-humoral) T cell phenotype.

Thymopoietin is a thymic hormone and an immune modulator that is known to be upregulated by the action of transcription factors E2F-4 and E2F-1 (Ren et al., 2002). The inventors describe for the first time upregulation of thymopoietin RNA in peripheral blood of MS patients. Of interest, analogs of thymopoietin (i.e., thymopentin) have been devised in an attempt to treat autoimmunity. Kantharia et al. (1989), has shown that thymopentin ameliorates rheumatoid arthritis. It is unknown if the elevation of thymopoietin in peripheral blood of MS is a compensatory phenomenon.

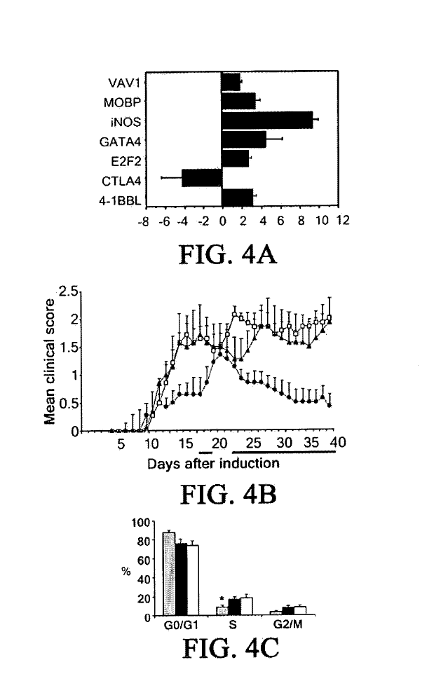

The Rb-E2F pathway plays a central role in the ability of immune cells to progress through the cell cycle and proliferate (Wu et al., 2001). Surprisingly, there are no previous reports implicating this pathway in multiple sclerosis, as disease-related reports of this pathway focus on cancer. The inventors show detection of significant upregulation of E2F-2, E2F-3, E2F-5, Rb, CDC25A, CDK2, BCL2 and DNA primase (PRIMI) in peripheral blood of MS patients. The mRNA expression of Rb, CDC25A and even E2Fs themselves is known to be dependent on E2F-mediated transcription, suggesting that indeed E2Fs are primarily elevated in MS peripheral blood, and not secondarily elevated to compensate for dysregulation of other pathways. As mentioned, thymopoietin mRNA is also elevated in the peripheral blood of MS patients and is similarly dependent on E2Fs for its transcription. Also in support of an upregulated E2F pathway, multiple histone mRNAs (identified by the gene descriptions H4, H2B.1, H2A2, H3.1, and H3.3) are disclosed in this invention to be upregulated in MS peripheral blood mononuclear cells, and E2F transcription factors are known to induce the expression of histones. In addition, histone deacetylase 1 (HD1, also known as HDAC1), a global regulator known to bind Rb, was upregulated. None of these gene products have been previously demonstrated to be upregulated in MS peripheral blood. The inventors therefore sought to validate these novel findings in the murine experimental autoimmune encephalomyelitis (EAE) animal model of MS. The results (FIGS. 4A-C; Table 19) indicate a role in EAE for an activated E2F pathway, which plays an essential role in the ability of cells to progress through the cell cycle (Wu et al., 2001) and has been shown to play a role in autoimmunity (Murga et al., 2001) but had not previously been associated with MS. The findings suggest that E2f pathway-mediated cell cycle progression is required to mount a vigorous proliferation response to an antigenic challenge that leads to CNS autoimmunity.

TABLE 18
Clinical Findings

| | EF21 | Wild-Type | C57BL/6 |
|---|---|---|---|
| Incidence | 7/7 | 7/7 | 5/7 |
| Day of Onset | 14.9 ± 1.9 | 13.1 ± 0.9 | 12.8 ± 1.3 |
| Mean Peak Remission Phase* | 1.3 ± 0.2 | 2.5 ± 0.2 | 3 ± 0.2 |
| Mean Percent Days of Severity* | 6.8 ± 4.4 | 48.2 ± 7.5 | 59.5 ± 6.6 |
| E2f1 deficient mice had a significant drop in the mean peak remission phase, | | | |
| #defined as the mean of the maximal score reached during remission, and in the | | | |
| #mean percentage of days of disease severity, defined as the mean % of days with | | | |
| #scores ≧ 2 (* = P < 0.002 by Fisher's PLSD). | | | |

RNA polymerase II, required for transcriptional activity, and DNA topoisomerase III, involved in DNA replication and the maintenance of genomic stability, were upregulated in MS peripheral blood (not previously reported) suggesting enhanced transcriptional and cell division activity. The inventors disclosed the upregulated expression of cyclin C, which regulates proliferation via its interactions with RNA polymerase II (Rickert, 1996), and of cyclin D2, which enhances Rb phosphorylation, and therefore S phase cell cycle entry. The inventors also disclose enhanced expression in MS peripheral blood of the T cell receptor engagement-induced activation marker CD27, and soluble CD27 has been previously reported to be elevated in cerebrospinal fluid from MS patients (Hintzen et al., 1991). The inventors report that the hypoxanthine guanine phosphoribosyl transferase (HPRT) gene is downregulated in MS peripheral blood, and it has been shown that this gene accumulates mutations in immune cells undergoing repeated cell divisions, a likely a mechanism accompanying autoimmunity. In fact, mutations in HPRT (which would theoretically impair binding of the mutated target RNA to the probe sets on these chips) have been demonstrated in peripheral blood T cells that have expanded to react against myelin basic protein in MS patients (Allegreta et al., 1990). Thus, the lower detection of HPRT in MS may be due theoretically to a mutated (i.e., mismatched) target RNA. An important response to mutations or DNA damage would be to upregulate DNA repair genes, and the inventors disclose Rad23A homolog as an upregulated biomarker in MS. Surprisingly, hMLH1, another DNA repair gene product, is disclosed to be downregulated in MS peripheral blood. Further, O6-methylguanine-DNA methyltransferase, which removes mutagenic O6 alkylguanine from DNA, is hereby shown by the inventors to be strikingly downregulated in peripheral blood of MS. Aberrant DNA repair mechanisms may play a role in perpetuating autoimmunity, since proliferating cells tend to accumulate mutations, and the immune system is known to become progressively more and more dysregulated. In accordance with this concept, defective repair of O6-methyl guanine has been associated with autoimmune diseases such as arthritis and systemic lupus erythematosus (SLE) (Lawley et al., 1988).

Another important set of biomarkers for MS, not previously reported in association with this disease, include the upregulated retinoic acid pathway genes RXR β, RAR-β2, and RAR-γ1, possibly involved in immune cell differentiation. Another important aspect of the invention is the identification of several homeobox transcription factors that are dysregulated in MS (Gbx2, NBPhox, and Hox4D, all upregulated, and Hox3D, found to be downregulated). These transcription factors are highly conserved through evolution and have not previously been implicated in MS. As further support of the clonal expansion hypothesis, the inventors show multiple markers of T cell markers and T cell receptor genes that are upregulated in MS peripheral blood. These include: CD8 β chain glycoprotein, TCRBV1S1A1N1, T cell receptor clone IGRA17, T cell receptor β-chain J2.1, T-cell receptor active β-chain from Jurkat cell line, and T cell receptor zeta chain. As evidence of B cell clonal expansion, the inventors show specific immunoglobulin (Ig) gene transcripts and Ig receptors to be upregulated in MS, including: Ig J chain, Rearranged Ig lambda light chain, α β autoantibody IgM heavy chain variable V-D-J region, Ig-like transcript 2, Ig-like transcript 3, omega light chain Ig lambda light chain related, Ig heavy chain Vdjc region, IgG low affinity Fc fragment receptor (FcRIIa), and the leukocyte IgG receptor (Fc-γ-R).

The inventors also disclose the finding of multiple surface molecules that participate in cell-cell interactions and are known to lead to immune cell activation. These include: upregulated CD28, LFA-1, ICAM-2,4-1BB ligand, CD40 ligand, and importantly, downregulated CTLA4 (CD152), an immune attenuator that when knocked out in mice leads to autoimmunity. Several reports have already described the potential association of CTLA4, CD28, CD40 ligand and other costimulatory molecules (such as LFA-1) with autoimmunity. Signaling molecules are also disclosed, including vav and vav2, from a family of genes that mediate T- and B-cell receptor activation and influence NFkB and JNK signaling. Mice that carry mutations in cbl-b, a gene that interacts with vav, are prone to autoimmunity. Vav family members also mediate signaling from the Fe γ receptor IIa1, and a probe set for this immunoglobulin receptor was also upregulated in MS peripheral blood.

Further, complex survival pathway mechanisms for immune cells may play a role in autoimmunity, and the inventors describe their finding of upregulated Bcl-2, fas and fas ligand genes (all three previously implicated in MS), and downregulated survival motor neuron protein in the peripheral blood of MS patients. The inventors also disclose CLARP, a pro-apoptotic gene not previously associated with MS, as upregulated in peripheral blood.

Supporting clonal expansion and/or differentiation of immune cells, the following markers were found to be upregulated: T cell surface antigen CD2 (T11), B lymphocyte antigen CD20, myeloid cell nuclear differentiation antigen, GM-CSF receptor and macrophage colony stimulating factor (M-CSF), and the myeloid cell surface marker CD11b. Upregulation of class I molecules is also disclosed, including MHC class I (HLA-A*8001), MHC class I—C, MHC class I CD8 α chain, β-2 microglobulin, and the MHC binding protein 2 (MBP2). The acute phase reactant c-reactive protein was upregulated, and this gene product has not been previously implicated in MS. Several interleukins (IL) or IL-receptors were found to serve as biomarkers of MS, and included: chemokine IL-8 receptor β, IL-6 receptor, IL-16 protein precursor, IL-3 receptor α subunit, IL-7 receptor, and the TGF-βII receptor α. The IL-6 receptor soluble form (not its peripheral blood immune cell RNA counterpart) has been reported to be highly elevated in serum and CSF of MS patients (Padberg et al., 1999; Stelmasiak et al., 2001). The IL-8 receptor is a chemotactic receptor that has been demonstrated to be elevated within MS plaque macrophages (Muller-Ladner et al., 1996). Taken together, upregulated IL-3 receptor, E2Fs and Bcl-2(all involved in the same IL-3 pro-survival pathway) indicate a direct involvement in MS-related autoimmunity for these biomarkers.

A striking and unexpected disclosure in this invention is the upregulated expression of myelin genes in peripheral blood mononuclear cells. This has not been described before as a factor in autoimmunity affecting the central nervous system. Since myelin is known to be expressed in the central or peripheral nervous systems, or in the thymus, but not in peripheral blood, the inventors disclose the upregulated myelin-component genes 2,3-cyclic nucleotide 3 phosphodiesterase (CNP), myelin basic protein (MBP), myelin-associated oligodendrocytic basic protein (MOBP) as potential triggers of autoimmunity in the peripheral circulation (the first demonstration of their expression and upregulation in peripheral blood mononuclear cells) and as novel biomarkers of the disease. Further supporting this invention, the peripheral myelin protein PMP-22 was not upregulated in peripheral blood of MS patients. The findings are novel since these gene products have previously been hypothesized to trigger autoimmunity in humans from the central nervous system compartment, not from their upregulated expression in the peripheral circulation. Furthermore, butyrophilin, an antigen present in cow milk and therefore proposed as an environmental antigen though to lead to molecular mimicry in MS (Winer et al., 2001) and which localizes within the HLA/complement region in human chromosome 6, is found by the inventors to be elevated in peripheral blood of MS patients. In addition, the inventors demonstrate higher expression in MS blood of a transcript representing the putative MS autoantigen L neurofilament (Eikelenboom et al., 2003). The expression in the blood of these self antigens, whether present in differentiating hematopoietic precursors or in antigen presenting cells, may play a role in priming and maintaining the autoimmune response that leads to demyelination in MS, and may serve as targets of treatment.

In addition, it has been shown that activation of immune cells leads to aberrant expression of endogenous retroviral sequences (Johnston et al., 2001). Consistent with these reports, the inventors find specific upregulation of the probe sets representing human endogenous retrovirus K10 (HERVK10)/HUMMTV reverse transcriptase homolog, HERVK10 neutral protease protein, ERV9 reverse transcriptase homolog, and a tigger 1 transposable element. The inventors also show that an endogenous retrovirus probe set (X72790) is downregulated in MS patients receiving Avonex, suggesting a beneficial mechanism for the drug.

Further, several signaling molecules are disclosed as biomarkers of MS. These include several probe sets for each of the following: centaurin, casein kinase I, and PI3 kinase, known to participate in the same signaling pathway, among many other signaling molecules. Finally, the inventors disclose two signaling biomarkers in MS peripheral blood that may serve as targets of treatment: FK13P-rapamycin associated protein (FRAP), upregulated in MS, and Rapamycin and FK506-binding protein FKBPI3, found to be downregulated in MS peripheral blood.

Another biomarker reported by the inventors to be elevated in peripheral blood of MS patients is natural resistance-associated macrophage protein-1 (NRAMP1), a molecule involved in iron metabolism and in the susceptibility to infections and/or autoimmunity. NRAMP1 promoter allelic variation has been significantly associated with MS susceptibility in the South African Caucasian population (Kotze et al., 2001) but it has not been shown to be elevated in peripheral blood previously. The inventors also disclose upregulation in MS peripheral blood of the mRNAs for Calgizzarin, a calcium-binding protein also known as SiOOC, and for another probe set representing SiOOD, which have been implicated in the regulation of cytoskeletal function, suggesting that these proteins may participate in the morphological changes observed in activated immune cells. Neither calgizzarin nor S100D have been previously associated with MS in peripheral blood. A recent report described Calgizzarin (probe set D38583) elevation in MS brain lesions as demonstrated by DNA microarrays (Lock et al., 2002). Interestingly in that study Lock reported upregulation in MS brain tissue of several other genes that were also found to be upregulated in MS peripheral blood by the inventors. These include Stat5B (U47686) and obese (D49487) (both found to be upregulated in acute/active MS plaques by Lock), and T-cell receptor b-chain J2.1 gene (M14159), p38 mitogen activated protein (MAP) kinase (L35253, known to interact in the vav pathways), platelet glycoprotein IIa (GPIIIa) (M35999), and platelet glycoprotein IIb (GPIIb) (M34344) (all these found to be upregulated in chronic/silent MS plaques by Lock). In addition, other genes upregulated in peripheral blood of MS patients that are disclosed by the inventors, and were also found by Lock to be regulated in MS brain tissue included CDKIO (X78342), two lysozyme probe sets (X14008 and M19045), HSI hematopoietic lineage cell specific protein (X16663), heterogeneous nuder ribonucleoprotein I (Hnrp I) (X66975), 5′-nucleotidase (D38524), histone deacetylase 1 (HDAC1) (D50405), histone H2A.2 (L19779), hypothetical protein 384D8_{—}7 (U62317) and a probe set for hypothetical protein A4 (U81556). Lock also reported multiple genes downregulated in MS brain tissue, some of which the inventors also disclose to be downregulated in peripheral blood of MS patients. These include: Thy-1 (M11749), Siah binding protein 1 (U51586), cytochrome c1 (J04444), and cytochrome c oxidase Va subunit (M22760). Finally, the cholecystokinin type A receptor (CCK-A) is a receptor previously implicated in pancreatic function and in the development of non-insulin dependent diabetes (Takiguchi et al., 1998), but not in MS: the inventors reveal its mRNA upregulation as a biomarker for multiple sclerosis.

Further supporting a role for oxidative stress in MS, the inventors have found that mitochondrial isocitrate dehydrogenase (NADP+) is downregulated in MS peripheral blood. Jo et al. (2001) have described an essential role for mitochondrial isocitrate dehydrogenase (NADP+) in preventing reactive oxygen species (ROS) generation. Therefore, this gene has potent antioxidant properties, and its downregulation in MS, in combination with elevated iNOS, may underlie oxidative stress-induced damage in this disease.

The inventors also demonstrate upregulation of the following genes in MS peripheral blood: the CUG repeat RNA-binding protein Etr-3 (probe set U69546), implicated in the regulation of splicing events (Ladd et al., 2001); the immunoglobulin superfamily member basal cell adhesion molecule (B-CAM); the adenomatous polyposis coli (APC) gene (represented in the microarray under the gene description “polyposis locus DP25”); a gene product known as LLGL (a regulatory target of the homeobox gene Hoxc8); the ubiquitin ligase cullin (CUL-2); the leukocyte antigen CD37, involved in regulating T cell-dependent B cell antibody responses (Knobeloch et al., 2000); and the ribosomal protein RPL37A. None of these biomarkers have previously been reported in association with MS.

KIAA0027 representing the membrane protein MLC1, KIAAOO10 representing an ubiquitin-protein isopeptide ligase, KIAA0128 representing septin 2, 6, and the KIAA0033 of yet unknown function, are also upregulated biomarkers for MS. In addition, probe sets representing uterus ficolin 1, nidogen (a basement membrane target of matrix metalloproteinases), the spliceosomal protein Sap62, the small G protein Gx, the G protein-coupled receptor v28, the transcription factor GATA-2 (probe set M68891) involved in immune cell differentiation, and the clone 23815 sequence (probe set U90916), are also disclosed as upregulated biomarkers for MS peripheral blood. In contrast, the α B-crystallin (a glioma Rosenthal fiber component) and the monocyte pseudoautosomal boundary mRNAs are both disclosed as downregulated biomarkers in MS peripheral blood mononuclear cells.

2. Treatments

Multiple sclerosis is an inflammatory, autoimmune disease that is known to be adversely affected by interferon γ (i.e., administration to MS patients in a clinical study lead to disease exacerbations), but on the contrary, is benefited by treatment with interferon β. The inventors included in their study a population of MS patients on treatment with interferon-β (Avonex) as a control, to assess reliability of their invention. The inventors were able to identify in vivo, genes previously reported to respond to interferon β in vitro (Der et al., 1998; U.S. Pat. No. 6,331,396). For instance, in accordance with Der's report of in vitro-stimulated cells, the inventors showed that MS patients on treatment with interferon β have peripheral blood upregulation of the interferon-responsive genes LMP2 and IP-30, and downregulation of the known interferon-downregulatable gene histone H4. Of note, both histone H4 and E2F-3 are transcriptionally activated by E2F proteins, and MS patients on Avonex also had downregulated E2F-3 mRNA as compared to non-treated MS patients, suggesting that the Rb/E2F pathway may be a target of Avonex. The inventors also identified other genes known to be interferon pathway-related as upregulated in vivo (but not shown in Der's report), including the interferon regulatory factor 7A and the interferon γ-inducible indoleamine 23-dioxygenase. Further, the inventors showed multiple other genes that are shown to be up- or downregulated in patients receiving interferon β, indicating that they are targets of treatment as well.

Finally, the inventors identify four genes that were upregulated in MS peripheral blood, but not significantly downregulated in vivo by Avonex; however, they may still represent valid targets of interferon β since it is now known that other commercial versions of interferon β may be more efficacious in vivo (i.e., Rebif and Betaseron), and they are shown in Der's report to be downregulated by interferon β but not by interferon γ. These four genes are clone Id2, ZnT-3, clone 23748, and a gene represented by accession number U82311. The inventors therefore disclose these genes specifically as therapeutic targets in MS.

III. Diagnostic/Prognostic Determinations in MS

The lists presented above can be used for determination of sample origin (ALS/healthy vs. MS). The lists above (except for Table 16) were generated by comparing the gene expression profile of peripheral blood mononuclear cells derived from 5 relapsing-remitting MS patients, 5 relapsing-remitting MS patients on treatment with Avonex (IFN-β1a), 5 ALS patients, and 5 healthy donors. As expected, the comparison of ALS vs. healthy donors yielded no significant differences, since ALS is not an autoimmune disease. However, every comparison involving MS patients peripheral blood yielded massive changes in gene expression, consistent with its autoimmune nature. The gene expression changes were separated into three classes:

(1) highest discriminatory, having a P value <0.05 by a new statistical method (see examples), regardless of the P value by T statistics (a much less stringent method);

(2) intermediate discriminatory: P value <0.01 by T statistics and >0.05 by the new method; and

(3) least discriminatory: P value <0.05 by T statistics and >0.05 by the new method.

Note that the least discriminatory genes may also be used for diagnostic purposes using microarrays or other methods (see below). In addition, since ALS samples did not differ in any significant manner from healthy donor samples, the gene lists in which both ALS and healthy donors are combined into a single group (to provide higher statistical validity) compared to MS are preferred in this invention for demonstrating MS discriminatory genes.

IV. Methods of Assaying for Alterations in Gene Expression

Thus, in accordance with the present invention, methods are provided for the assaying of gene expression in patients suffering from or at risk of MS. As discussed above, the principal applications of this assay are to: (a) identify patients whose gene expression profile puts them at risk of developing MS; identify patients whose symptoms are such that they may or may not be suffering from MS (i.e., provide a definitive diagnosis of MS); and (c) assess the impact of an MS therapy. In each of these assays, the expression of a particular set of genes, set forth in the preceding sections, will be measured. The following is a discussion of various aspects of these methods.

1. Hybridization

There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCRTM, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Since many nucleic acids, especially mRNAs, are in low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCRTM) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers”, the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller and Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohiman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA or total RNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501,5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

V. Protein-Based Diagnostic Assays

In another aspects of the invention, one may employ a protein-based diagnostic approach. The most common form of protein identification is by the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference).

In accordance with the present invention, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev 0, 1999; Gulbis and Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove nonspecifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies. “Under conditions effective to allow immune complex (antigen/antibody) formation” means that the conditions preferably include diluting the antigens and/or antibodies with solutions such as BSA, bovine ≡ globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

VI. Gene Therapy

In another embodiment, the present invention provides for the administration of a gene therapy vector encoding one or more genes identified as being downregulated in MS. Alternatively, for genes that are overexpressed in MS, the transgenes may provide for reduced expression of appropriate targets. Various aspects of gene delivery and expression are set forth below.

1. Therapeutic Transgenes

Thus, in accordance with the present invention, there are provided methods of treating MS utilizing genes identified as being overexpressed or underexpressed in MS. By inhibiting or increasing the expression of various of these genes, therapeutic benefit may be provided to patients.

2. Antisense

The term “antisense” nucleic acid refers to oligo- and polynucleotides complementary to bases sequences of a target DNA or RNA. When introduced into a cell, antisense molecules hybridize to a target nucleic acid and interfere with its transcription, transport, processing, splicing or translation. Targeting double-stranded DNA leads to triple helix formation; targeting RNA will lead to double helix formation.

Antisense constructs may be designed to bind to the promoter or other control regions, exons, introns or even exon-intron boundaries of a gene. Antisense RNA constructs, or DNA encoding such antisense RNA's, may be employed to inhibit gene transcription or translation within a host cell. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine in the case of DNA (A:T), or uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” and “antisense sequences” mean nucleic acid sequences that are substantially complementary over their entire length and have very few base mismatches. For example, nucleic acid sequences of fifteen bases in length may be termed complementary when they have complementary nucleotides at thirteen or fourteen positions. Naturally, nucleic acid sequences with are “completely complementary” will be nucleic acid sequences which have perfect base pair matching with the target sequences, i.e., no mismatches. Other sequences with lower degrees of homology are contemplated. For example, an antisense construct with limited regions of high homology, but overall containing a lower degree (50% or less) total homology, may be used.

While all or part of the gene sequence may be employed in the context of antisense construction, statistically, any sequence of 17 bases long should occur only once in the human genome and, therefore, suffice to specify a unique target. Although shorter oligomers are easier to make and increase in vivo accessibility, numerous other factors are involved in determining the specificity of hybridization: Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting a gene simply by testing the construct in vitro to determine whether the gene's function or expression is affected.

In certain embodiments, one may wish to employ antisense constructs which include other elements, for example, those which include C-5 propyne pyrimidines. Oligonucleotides which contain C-5 propyne analogs of uridine and cytidine have been shown to bind RNA with high affinity and to be potent inhibitors or gene expression. Wagner et al. (1993).

3. Ribozymes

The term “ribozyme” refers to an RNA-based enzyme capable of targeting and cleaving particular DNA and RNA sequences. Ribozymes can either be targeted directly to cells, in the form of RNA oligonucleotides incorporating ribozyme sequences, or introduced into the cell as an expression construct encoding the desired ribozymal RNA. Ribozymes may be used and applied in much the same way as described for antisense nucleic acids. Ribozyme sequences also may be modified in much the same way as described for antisense nucleic acids. For example, one could include modified bases or modified phosphate backbones to improve stability or function.

4. RNA Interference

RNA interference (RNAi) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNAᵢ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al. (1999). Moreover, dsRNA has been shown to silence genes in a wide range of systems, including plants, protozoans, fungi, C. elegans, Trypanosoma and Drosophila. Grishok et al. (2000); Sharp (1999); Sharp & Zamore (1999).

Several principles are worth note (see Plasterk & Ketting, 2000) First, the dsRNA should be directed to an exon, although some exceptions to this rule have been shown. Second, a homology threshold (probably about 80-85% over 200 bases) is required. Most tested sequences are 500 base pairs or greater. Third, the targeted mRNA is lost after RNAᵢ. Fourth, the effect is non-stoichometric, and thus incredibly potent. In fact, it has been estimated that only a few copies of dsRNA are required to knock down >95% of targeted gene expression in a cell. Fire et al. (1998). Recently, shorter (˜20 base pairs) synthetic duplex RNAs have been shown to efficiently perform RNAi, by using liposome transfection. Further, similar short interfering RNA (siRNA) duplexes of 19-25 base pairs have been used by transfection via recombinant DNA constructs containing a promoter for U6 small nuclear RNA (snRNA) to drive nuclear expression of a single RNA transcript. This is also known as the hairpin siRNA/suppression of endogenous RNA (SUPER) strategy and has been shown to eliminate the expression of a target gene in longterm mammalian cell cultures (Brummelkamp et al., Science 2002; 296: 550-553; Paul et al., Nature Biotechnol 2002; 20: 505-508.; Lee et al., Nature Biotechnol 2002; 19: 500-505; Miyagishi et al., Nature Biotechnol 2002; 19: 497-500).

Although the precise mechanism of RNAᵢ is still unknown, the involvement of permanent gene modification or the disruption of transcription have been experimentally eliminated. It is now generally accepted that RNAᵢ acts post-transcriptionally, targeting RNA transcripts for degradation. It appears that both nuclear and cytoplasmic RNA can be targeted. Bosher and Labouesse (2000).

5. Single Chain Antibodies

Naturally-occurring antibodies (of isotype IgG) produced by B cells, consist of four polypeptide chains. Two heavy chains (composed of four immunoglobulin domains) and two light chains (made up of two immunoglobulin domains) are held together by disulphide bonds. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (VH and VL) at the ends of the arms. Each variable domain contains three hypervariable regions known as complementarity determining regions, or CDRs. The CDRs come together in the final tertiary structure to form an antigen binding pocket. The human genome contains multiple fragments encoding portions of the variable domains in regions of the immunoglobulin gene cluster known as V, D and J. During B cell development these regions undergo recombination to generate a broad diversity of antibody affinities. As these B cell populations mature in the presence of a target antigen, hypermutation of the variable region takes place, with the B cells producing the most active antibodies being selected for further expansion in a process known as affinity maturation.

A major breakthrough was the generation of monoclonal antibodies, pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody. It may be better to deliver antibodies by gene therapy, as this would hopefully provide a constant localized supply of antibody following a single dose of vector. The problems of vector design and delivery are dealt with elsewhere, but antibodies in their native form, consisting of two different polypeptide chains which need to be generated in approximately equal amounts and assembled correctly are not good candidates for gene therapy. However, it is possible to create a single polypeptide which can retain the antigen binding properties of a monoclonal antibody.

The variable regions from the heavy and light chains (VH and VL) are both approximately 110 amino acidg long. They can be linked by a 15 amino acid linker (e.g., (glycine₄serine)₃), which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity. However, there is evidence that scFvs spontaneously multimerize, with the extent of aggregation (presumably via exposed hydrophobic surfaces) being dependent on the length of the glycine-serine linker.

The variable regions for constructing the scFv are obtained as follows. Using a monoclonal antibody against the target of interest, it is a simple procedure to use RT-PCR to clone out the variable regions from mRNA extracted from the parent hybridoma. Degenerate primers targeted to the relatively invariant framework regions can be used. Expression constructs are available with convenient cloning sites for the insertion of the cloned variable regions.

6. Vectors

In accordance with the present invention, both stimulatory and inhibitory genes may be provided to cells of an MS patient and expressed therein. Stimulatory genes are generally simply copies of the gene of interest, although in some cases they may be genes, the expression of which direct the expression of the gene of interest. Inhibitory genes, discussed above, may include antisense or single-chain antibody genes.

The term “vector” is used to refer to a carrier nucleic acid molecule into which a nucleic acid sequence can be inserted f6r introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1989 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 18 lists non-limiting examples of elements/promoters that may be employed, in the context of the present invention, to regulate the expression of a RNA. Table 19 provides non-limiting examples of inducible elements, which are regions of a nucleic acid sequence that can be activated in response to a specific stimulus.

TABLE 18
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| lnterleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick et al., 1989; Johnson et al., |
| (MCK) | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulaled Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 19
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; |
| mammary tumor | | Lee et al., 1981; Majors et |
| virus) | | al., 1983; Chandler et al., |
| | | 1983; Lee et al., 1984; |
| | | Ponta et al., 1985; Sakai et |
| | | al., 1988 |
| β-Interferon | Poly(rI)x | Tavrnier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse α2 (XI) collagen (Tsumaki et al, 1998), D1A dopamine receptor gene (Lee et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al, 1992; Curiel, 1994). Adeno-associated virus (AAV) is an attractive vector system as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

7. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel and Baltimore, 1987), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al, 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

a. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

b. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

c. Calcium Phosphate

In other embodiments of the present invention, a nucleic acid may be introduced to the cells using calcium phosphate precipitation in an ex vivo context. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

d. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

e. Sonication Loading

Additional embodiments of the present invention include the introduction of a nucleic acid by direct sonic loading. LTK-fibroblasts have been transfected with the thymidine kinase gene by sonication loading (Fechheimer et al., 1987).

f. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

g. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

h. Microprojectile Bombardment

Microprojectile bombardment techniques can be used to introduce a nucleic acid ex vivo into at least one, organelle, cell, or tissue (U.S. Pat. Nos. 5,550,318, 5,538,880, 5,610,042, and PCT Application WO 94/09699; each of which is incorporated herein by reference). This method depends on the ability to accelerate DNA-coated microprojectiles to a high velocity allowing them to pierce cell membranes and enter cells without killing them (Klein et al, 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

VII. Pharmaceutical Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the present invention will be via any common route so long as the target tissue is available via that route. This includes intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

RNA isolation and hybridization of biotin-labeled cRNA to HuFL6800 GeneChips. Peripheral blood obtained from MS patients, ALS patients and healthy donors was subjected to Ficoll-paque gradient centrifugation for isolation of whole mononuclear cells (also known as peripheral blood mononuclear cells or PBMCs). RNA was then isolated using the Trizol (Gibco BRL) method. The inventors used protocols for cRNA preparation and biotin labeling for use with the Affymetrix HuFL6800 microarrays that have been published (Warrington et al., 2000). Briefly, the method is described below:

The HuFL6800 microarrays contain 7,070 distinct probe sets, representing approximately 6,800 human genes. Briefly, using 7-10 μg of total RNA, double stranded cDNA was synthesized using the Superscript Choice System (Life Technologies) with the following modifications. In the first strand synthesis, the reverse transcription reaction contained a T7(dT)₂₄ primer plus 0.1 M DTT and 10 mM dNTP mix. For second strand synthesis, E. coli DNA ligase (10 U/μl) and T4 DNA Polymerase I (10 U/μl), 10 mM dNTP mix and RNase H (2 U/μl) were used. Phenol-chloroform extraction was followed by in vitro transcription (IVT) (Enzo Bioarray™ High Yield™ RNA transcript labeling kit, distributed by Affimetrix, Inc.) with biotin labeling. IVT was performed with (1:3) biotinylated: unlabeled CTP and UTP. The T7 enzyme mix and T7 transcription buffer were added to the ds cDNA and NTP labeling mix (ATP, CTP, UTP, GTP, Bio-11-CTP and Bio-16-UTP). The NTP labeling mix was incubated for 5 hr at 37° C., and cleaned using RNeasy columns (Qiagen). 13-20 μg of fluorescently-labeled and chemically-fragmented cRNA were used for array hybridization. Fragmented cRNA and herring sperm DNA were added to the hybridization buffer containing 1.0 M NaCl, 10 mM Tris-HCL pH 7.6, and 0.01% Triton X-100.

The hybridization mixture was heated to 99° C. for 5 min., spun, and incubated at 45° C. for 5 min, and injected into the probe array cartridge. Hybridizations were carried out at 45° C. for 16 hours with mixing at 60 rpm. Following hybridization, solutions were removed, and arrays were rinsed and incubated with 0.1×ST-T (100 mM NaCl, 10 mM Tris-HCL pH 8.0, and 0.01% Triton X-100) at 50° C. for 20 min. Hybridized arrays were stained with 5.0 μg/ml streptavidin-phycoerythrin (Molecular Probes) and 2.0 mg/ml acetylated BSA (Sigma) in 1×ST-T at 40° C. for 15 min. The streptavidin-phycoerythrin step was repeated after an intermediate amplification step in which anti-streptavidin rabbit IgG antibodies and secondary biotinylated goat anti-rabbit antibodies are added to the samples. Following washes, probe arrays were scanned twice at 6 μm resolution using the GeneChip system confocal scanner.

Analysis was performed using Affymetrix Microarray Suite software, which assesses presence or absence of transcripts for each probe set, taking into account metrics such as background, noise, and comparison of intensities between Perfect Match (PM) and their control Mismatch (MM) probe cells. The average intensity of each microarray was scaled to a target intensity of 1500. Prior to statistical analysis, all average difference intensity values of less than 20 and all negative values were assigned a set value of 20. Results from each sample were grouped into a defined class (i.e., MS, MS on Avonex, ALS, and healthy donors).

Selection of Discriminatory Genes. Several statistical measures have been introduced to identify discriminatory genes for two conditions (e.g., cancerous and normal tissues). Parametric tests such as P-value (Golub et al., 1999) and t-test (Thomas et al. 2001) are based on differences of group means, while non-parametric tests such as Wilcoxon rank sum (MannWhitney) test are based on differences of rank sums in groups (Thomas et al., 2001). A couple of measures such as Wilks' lambda were also proposed for the identification of discriminatory genes in multi-classes (Dudoit et al., 2001). All these measures have their own advantages and disadvantages and no method is thus unanimously optimal for all kinds of data. Parametric tests may perform poorly due to violation of their underlying assumptions, such as normality and equal variance in the various groups. A non-parametric test does not rely on these assumptions and works well with a small sample size, but the results may be more critically sensitive on the nature of the samples used for the training of the classifier than those in parametric tests. For these reasons, many methods normally yield various levels of false positive discriminatory genes, depending on the degree of how the method used violates the underlying assumptions for a given data set and is sensitive to the sample size.

A few methods such as Significance Analysis of Microarrays (SAM) (Storey et al., 2001; Tusher et al., 2001) have been introduced to reduce false positive error rate in identifying discriminatory genes. These methods do not directly estimate the probability of false positive errors to calculate a false positive error rate. However, they introduce both a typical measure (e.g., simple t-test) to produce a large set of discriminatory genes including many false positives, and a more stringent measure (e.g., permutation based SAM) to produce a smaller set of genes only with high discriminatory characteristics. A false discovery rate is then indirectly estimated based on the reduction of the size of the latter set of discriminatory genes, with respect to the former set. Here, the inventors introduce a novel method for discriminatory gene selection that directly estimates the probability of false positive error for a given false negative error, thus more precisely eliminating false positives. This method involves a standard statistical test (e.g., t-test) on the basis of the distribution of the null hypothesis (Ho: group means are same), but the unique feature in this method is to include additionally the distribution of the alternative hypothesis (H₁: group means are not same). Normal t-test involves the hypothesis testing with mean difference scaled by a pooled standard deviation, based on only the distribution of H₀ that the scaled mean differences follow, when the group means are same. For a given discriminatory gene whose means are distinct in classes, the scaled mean difference stays outside the H₀ distribution. The normal t-test calculates the probability of observing the given scaled mean difference by chance, when H₀ is true (e.g., the group means are same) (Rice, 1995; Statistics toolbox Version 2.1.1, 1997). If this probability, called significance, is less than a desired significance level (α=0.05 or 0.01), then reject the H₀ and accept the H₁, concluding that the gene is discriminatory. However, this significance is only calculated in the view of H₀, never involving the H₁ distribution. Thus, the probability that the gene is actually discriminatory (i.e., H₁ is true) is not calculated before the H₁ is accepted. In this light, to identify a more correct set of discriminatory genes, both distributions H₀ and H₁ should be simultaneously considered.

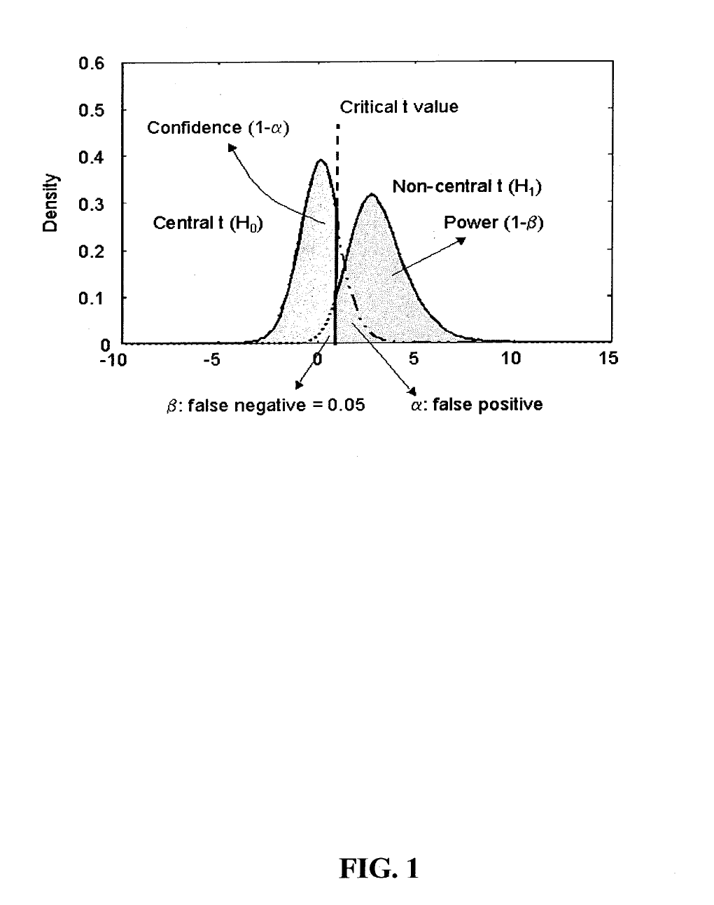

Considering the two distributions together, there are two types of errors in the standard hypothesis testing: false positive (α) and false negative (β) errors (two tailed t-test; see FIG. 1). False positive error is defined by the probability that the gene is in fact not discriminatory (i.g., H₀ is true), but the test concludes that the gene is discriminatory (i.e., H₁ is true), while false negative error is by the probability that the gene is in fact discriminatory, but the test concludes that the gene is not discriminatory (i.g., H₀ is true) (see FIG. 1). This novel method involving these two distributions comprises two steps to determine false positive error for each gene with a given false negative error and select discriminatory genes whose estimated false positive errors are less than a certain cutoff value (α=0.05). First, the H₀ and H₁ distributions are first defined. While the H₀ distribution in the standard t-test can be used, a non-centrality parameter (Δ) is required to define the H₁ distribution, as shown in Equations 1 to 3.

H₀:μ₁=μ₂ and H₁:μ₂≠μ₂ (1)

$$\begin{matrix}{H}_{0} : t = \frac{( {\overline{x}}_{1} - {\overline{x}}_{2} )}{{S}_{p} \sqrt{1 / {n}_{1} + 1 / {n}_{2}}} ∼ t ( N - 2 ) & ( 2 ) \\ {H}_{1} : t = \frac{( {\overline{x}}_{1} - {\overline{x}}_{2} )}{{S}_{p} \sqrt{1 / {n}_{1} + 1 / {n}_{2}}} ∼ t ( N - 2 ; Δ = \frac{{( {\overline{x}}_{1} - {\overline{x}}_{2} )}_{actual}}{{S}_{p} \sqrt{1 / {n}_{1} + 1 / {n}_{2}}} ) & ( 3 )\end{matrix}$$

where the bar of xᵢ is the mean in the i-th class and Sₚ is the pooled standard deviation. N is the total number of samples and nᵢ is the number of samples belonging to the i-th class. t(N-2) and t(N-2; Δ) are a central t-distribution with the degrees of freedom (d.o.f.) of N-2 and a non-central t-distribution with the same d.o.f. and the non-centrality parameter Δ. For each gene, the actual scaled mean difference is used in Equation 3 to estimate the non-centrality parameter Δ. Then, by taking the inverse of the H₁ distribution, a critical t statistic value is calculated for the given false negative error (β=0.05) (see FIG. 1). Second, a false positive error for each gene can be calculated using the H₀ distribution with this critical t value (see Equation 1 and FIG. 1). If this estimated false positive error is less than a certain cutoff value (α=0.05), the corresponding gene is selected as a discriminatory gene (see Discriminatory gene lists). The genes selected in this way are denoted by a set of most discriminatory genes. This procedure is schematically depicted in FIG. 1.

The standard t-test is also used to identify a set of moderately discriminatory genes and a set of less discriminatory genes. The moderately discriminatory genes are selected by a cutoff value of 0.01 for the significances of genes in the t-test, while the less discriminatory genes are by a cutoff value of 0.05.

Fisher Discriminant Analysis (FDA). Fisher Discriminant Analysis (FDA) is a linear method of dimensionality reduction from the expression space comprising all selected discriminatory genes to just a few dimensions where the separation of sample classes is maximized. FDA is similar to Principal Component Analysis (PCA) (Alter, 2000; Holter et al., 2000) in the linear reduction of data (Johnson & Wichem, 1992; Dillon & Goldstein, 1984). The major difference is that the discriminant axes of the FDA space are selected such as to maximize class separation in the reduced FDA space, instead of variability as in the case of PCA. The discriminant axes of FDA, termed as discriminant weights (V), maximizing the separation of sample classes in their projection space can be shown to be equivalent to the eigenvectors of W ¹B, the ratio of between-group variance (B) to within-group variance (W), as shown in Equation 4:

$${W}^{- 1} BV = V Λ \\ where B = T - W , W = {\sum }_{j = 1}^{c} {( {X}_{j} - 1 {\overline{x}}_{j}^{T} )}^{T} ( {X}_{j} - 1 {\overline{x}}_{j}^{T} ) , \\ and T = {( X - 1 {\overline{x}}^{T} )}^{T} ( X - 1 {\overline{x}}^{T} ) . \qquad ( 4 )$$

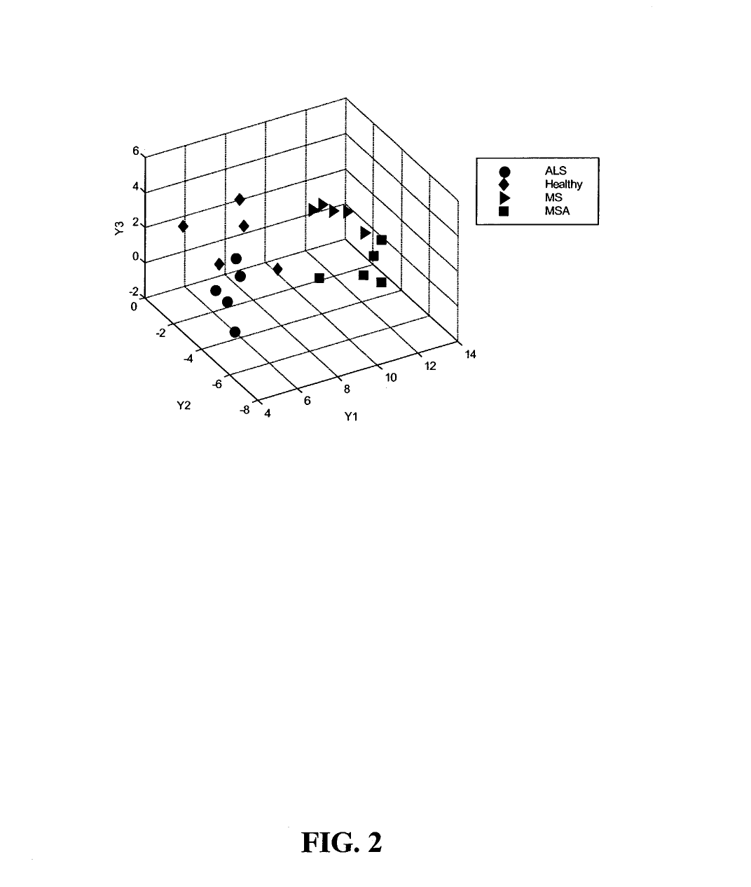

The eigenvalues (Λ) indicate the discrimination power for the corresponding discriminant axes. FIG. 2 shows the projection of the expression data in the 4-classes (ALS, Healthy, MS, and MS+Avonex). A classification rule can be built in the FDA space. A new sample is projected into the FDA space using the discriminant weights (V). Then, the new sample will be assigned to the predefined class whose mean is closest to the projection of the new sample (Johnson & Wichem, 1992): a new sample (x) will be allocated to class j if

∥ŷ−\overline{y}ⱼ∥²=(\hat{x}−\overline{x}ⱼ)V∥²≦∥(\hat{x}−\overline{x}ₖ)V∥² for all k≠j (5)

where ŷ is a projection of the new sample into the discriminant axes (V).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,683,195

U.S. Pat. No. 4,683,202

U.S. Pat. No. 4,684,611

U.S. Pat. No. 4,797,368

U.S. Pat. No. 4,800,159

U.S. Pat. No. 4,883,750

U.S. Pat. No. 4,952,500

U.S. Pat. No. 5,139,941

U.S. Pat. No. 5,242,974

U.S. Pat. No. 5,279,721

U.S. Pat. No. 5,302,523

U.S. Pat. No. 5,322,783

U.S. Pat. No. 5,384,253

U.S. Pat. No. 5,384,261

U.S. Pat. No. 5,405,783

U.S. Pat. No. 5,412,087

U.S. Pat. No. 5,424,186

U.S. Pat. No. 5,429,807

U.S. Pat. No. 5,436,327

U.S. Pat. No. 5,445,934

U.S. Pat. No. 5,464,765

U.S. Pat. No. 5,472,672

U.S. Pat. No. 5,527,681

U.S. Pat. No. 5,529,756

U.S. Pat. No. 5,532,128

U.S. Pat. No. 5,538,877

U.S. Pat. No. 5,538,880

U.S. Pat. No. 5,545,531

U.S. Pat. No. 5,550,318

U.S. Pat. No. 5,554,501

U.S. Pat. No. 5,556,752

U.S. Pat. No. 5,561,071

U.S. Pat. No. 5,563,055

U.S. Pat. No. 5,571,639

U.S. Pat. No. 5,580,859

U.S. Pat. No. 5,589,466

U.S. Pat. No. 5,591,616

U.S. Pat. No. 5,593,839

U.S. Pat. No. 5,599,695

U.S. Pat. No. 5,610,042

U.S. Pat. No. 5,624,711

U.S. Pat. No. 5,656,610

U.S. Pat. No. 5,658,734

U.S. Pat. No. 5,700,637

U.S. Pat. No. 5,702,932

U.S. Pat. No. 5,736,524

U.S. Pat. No. 5,780,448

U.S. Pat. No. 5,789,215

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,336

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,565

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,981,274

U.S. Pat. No. 5,994,136

U.S. Pat. No. 5,994,624

U.S. Pat. No. 6,004,755

U.S. Pat. No. 6,013,516

U.S. Pat. No. 6,331,396

Abbondanzo, Ann. Diagn. Pathol., 3(5):318-327, 1999.

Allegreta et al, Science, 247(4943):718-21, 1990.

Allred et al., Arch Surg, 125(1):107-13, 1990.

Almendro et al., J Immunol., 157:5411-5421, 1996.

Alter et al., Proc. Nat'l Acad. Sci. USA, 97, 10101-10106 (2000).

Angel et al., Mol. Cell. Biol., 7:2256, 1987.

Angel et al., Cell, 49:729, 1987b.

Angel et al., Mol. Cell Biol., 7:2256, 1987a.

Atchison and Perry, Cell, 46:253, 1986.

Atchison and Perry, Cell, 48:121, 1987.

Bagasra et al., Proc. Nat'l Acad. Sci. USA, Dec. 19, 1995;92(26):12041-5

Bagasra et al., Proc. Nat'l Acad. Sci. USA, 92(26):12041-5, 1995.

Baichwal and Sugden, In: Gene Transfer, Kucherlapati (ed.), New York, Plenum Press, 117-148, 1986.

Banerji et al., Cell, 27(2 Pt 1):299-308, 1981.

Banerji et al., Cell, 33(3):729-740, 1983.

Barcellos et al., Immunogenetics, 51: 281, 2000.

Berkhout et al., Cell, 59:273-282, 1989.

Blanar et al., EMBO J, 8:1139, 1989.

Blomer et al., J. Virol., 71(9):6641-6649, 1997.

Bo et al., Ann. Neurol., 36(5):778-86, 1994.

Bodine and Ley, EMBO J., 6:2997, 1987.

Boshart et al., Cell, 41:521, 1985.

Bosher and Labouesse, Nat. Cell Biol. 2(2):E31-36, 2000.

Bosze et al., EMBO J, 5(7):1615-1623, 1986.

Braddock et al., Cell, 58:269, 1989.

Brown et al., Immunol Ser, 53:69-82, 1990.

Bulla and Siddiqui, J. Virol., 62:1437, 1986.

Campbell and Villarreal, Mol. Cell. Biol., 8: 1993, 1988.

Campere and Tilghman, Genes and Dev., 3:537, 1989.

Campo et al., Nature, 303:77, 1983.

Carbonelli et al., FEMS Microbiol Lett, 177(1):75-82, 1999.

Carlin et al., J. Neuropathol. Exp. Neurol. 59:361, 2000.

Celander and Haseltine, J. Virology, 61:269, 1987.

Celander et al., J. Virology, 62:1314, 1988.

Chamberlan et al., In: PCR Protocols, eds. Innis, Gelfand, Sninsky, White (Academic Press, NY, 272-281, 1990.

Chandler et al., Cell, 33:489, 1983.

Chang et al., Mol. Cell. Biol., 9:2153, 1989.

Chataway et al., Neurogenetics 2:91, 1999.

Chatterjee et al., Proc Nat'l Acad. Sci. U.S.A., 86:9114, 1989.

Chen and Okayama, Mol. Cell Biol., 7(8):2745-2752, 1987.

Choi et al., Cell, 53:519, 1988.

Cocea, Biotechniques, 23(5):814-816, 1997.

Cohen et al., J. Cell. Physiol., 5:75, 1987.

Costa et al., Mol. Cell. Biol., 8:81, 1988.

Cotten et al., Proc Nat'l Acad Sci USA, 89(13):6094-6098, 1992.

Coupar et al., Gene, 68:1-10, 1988.

Cripe et al., EMBO J, 6:3745, 1987.

Culotta and Hamer, Mol. Cell. Biol., 9:1376, 1989.

Curiel, Nat. Immun., 13(2-3):141-64, 1994.

Dandolo et al., J. Virology, 47:55-64, 1983.

De Groot et al., J. Neuropathol. Exp. Neurol. 56(1):10-20, 1997.

De Jager et al., Semin. Nucl. Med. 23(2):165-179, 1993.

De Villiers et al., Nature, 312(5991):242-246, 1984.

Der et al., Proc. Nat'l Acad. Sci. USA 95:15623-8, 1998.

Deschamps et al., Science, 230:1174-1177, 1985.

Dillon and Goldstein, Multivariate Analysis, Wiley, New York; 1984.

Ding et al., J. Immunol., 160(6):2560-4, 1998.

Doolittle et al., Methods Mol. Biol., 109:215-237, 1999.

Dudoit et al., UC Berkeley Statistics tech. Report, 2001.

Edbrooke et al., Mol. Cell. Biol., 9:1908, 1989.

Edlund et al., Science, 230:912-916, 1985.

Eikelenboom et al., Neurology 60, 219-223, 2003.

European Application No. 0 364 255

European Application No. 320 308

European Application No. EPO 0273085

European Application No. GB 2 202 328

European Application No. 329 822

Fechheimer et al., Proc. Nat'l Acad. Sci. USA, 84:8463-8467, 1987.

Feng and Holland, Nature, 334:6178, 1988.

Femandez-Arquero et al Neurology 53:1361, 1999.

Firak and Subramanian, Mol. Cell. Biol., 6:3667, 1986.

Fire et al., Nature, 391:806-811, 1998.

Fiten et al., J. Neuroimmunol. 95:195, 1999.

Foecking and Hofstetter, Gene, 45(1):101-105, 1986.

Fraley et al., Proc. Nat'l Acad. Sci. USA, 76:3348-3352, 1979.

Friedmann, Science, 244:1275-1281, 1989.

Frohman, In: PCR Protocols: A Guide To Methods And Applications, Academic Press, N.Y., 1990.

Fujita et al., Cell, 49:357, 1987.

Fukazawa et al., J Neurol. Sci. 166: 47, 1999.

Fukazawa et al., J Neurol. Sci. 171: 49, 1999.

Gade-Andavolu et al., Arch. Neurol. 55:513, 1998.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gilles et al., Cell, 33:717, 1983.

Giovannoni et al., J Neurol. Sci., 145(1):77-81, 1997.

Gloss et al., EMBO J., 6:3735, 1987.

Godbout et al., Mol. Cell. Biol., 8:1169, 1988.

Golub et al., Science 286, 531-537, 1999.

Goodbourn and Maniatis, Proc. Nat'l Acad. Sci. USA, 85:1447, 1988.

Goodbourn et al., Cell, 45:601, 1986.

Gopal, Mol. Cell Biol., 5:1188-1190, 1985.

Graham and Van Der Eb, Virology, 52:456-467, 1973.

Greene et al., Immunology Today, 10:272, 1989

Grishok et al., Science, 287:2494-2497, 2000.

Grosschedl and Baltimore, Cell, 41:885, 1985.

Gulbis and Galand, Hum. Pathol. 24(12):1271-1285, 1993.

Harland and Weintraub, J. Cell Biol., 101:1094-1099, 1985.

Hashimoto et al., J Neuroimmunol. 44:77, 1993.

Haslinger and Karin, Proc. Nat'l Acad. Sci. USA, 82:8572, 1985.

Hauber and Cullen, J. Virology, 62:673, 1988.

Hen et al, Nature, 321:249, 1986.

Hensel et al., Lymphokine Res., 8:347, 1989.

Herr and Clarke, Cell, 45:461, 1986.

Hintzenetal., JNI, 35:211-7, 1991.

Hirochika et al., J. Virol., 61:2599, 1987.

Hirsch et al., Mol. Cell. Biol., 10:1959, 1990.

Holbrooketal., Virology, 157:211, 1987.

Holter et al., Proc. Nat'l Acad. Sci. USA 97, 8409-8414, 2000.

Horlick and Benfield, Mol. Cell. Biol., 9:2396, 1989.

Horwich et al., Virol., 64:642-650, 1990.

Huang et al., Cell, 27:245, 1981.

Hug et al., Mol. Cell. Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol., 4:875, 1984.

Innis et al., Proc. Nat'l Acad. Sci. USA 85(24):9436-9440, 1988.

Inouye and Inouye, Nucleic Acids Res., 13: 3101-3109, 1985.

Jacobsen et al., Nat. Genetics 26:495, 2000.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Jo et al., J. Biol. Chem., 276(19):16168-76, 2001.

Johnson and Applied Multivariate Statistical Analysis, Prentice Hall, New Jersey, 1992.

Johnson et al., Mol. Cell. Biol., 9:3393, 1989.

Johnston et al., Ann. Neurol., 50(4):434-42, 2001.

Kadesch and Berg, Mol. Cell. Biol., 6:2593, 1986.

Kaeppler et al., Plant Cell Reports 9: 415-418, 1990.

Kaneda et al., Science, 243:375-378, 1989.

Kantharia et al., Br. J. Rheumatol., 28(2):118-23, 1989.

Karin et al., Mol. Cell. Biol., 7:606, 1987.

Karin et al., Mol. Cell. Biol., 7:606, 1987.

Katinka et al., Cell, 20:393, 1980. Kato et al., J. Biol. Chem., 266:3361-3364, 1991.

Kawamoto et al., Mol. Cell. Biol., 8:267, 1988.

Kelleher and Vos, Biotechniques, 17(6):1110-7, 1994.

Ketting et al., Cell, 99:133-141, 1999.

Kiledjian et al., Mol. Cell. Biol., 8:145, 1988.

Klamut et al., Mol. Cell. Biol., 10: 193, 1990.

Klein et al., Nature, 327:70-73, 1987.

Knobeloch et al., Mol. Cell. Biol., 20(15):5363-9, 2000.

Koch et al., Mol. Cell. Biol., 9:303, 1989.

Kotze et al., Blood Cells Mol. Dis., 27(1):44-53, 2001.

Kraus et al., FEBS Lett., 428(3):165-170, 1998.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kuhl et al., Cell, 50:1057, 1987.

Kunz et al., Nucl. Acids Res., 17:1121, 1989.

Kwoh et al., Proc. Nat'l Acad. Sci. USA, 86: 1173, 1989.

Ladd et al., Mol. Cell Biol., 21(4):1285-96, 2001.

Lareyre et al., J. Biol. Chem., 274(12):8282-8290, 1999.

Larsen et al., Proc Nat'l Acad. Sci. USA., 83:8283, 1986.

Laspia et al., Cell, 59:283, 1989.

Latimer et al., Mol. Cell. Biol., 10:760, 1990.

Laughlin et al., J. Virol., 60(2):515-524, 1986.

Lawley et al., Ann. Rheum. Dis., 47(6):445-51, 1988.

Lebkowski et al., Mol. Cell. Biol., 8(10):3988-3996, 1988.

Lee et al., J. Auton. Nerv. Syst. 74(2-3):86-90, 1997.

Lee et al., Nature, 294:228, 1981.

Lee et al., Nucleic Acids Res., 12:4191-206, 1984.

Levenson et al., Hum Gene Ther, 9(8):1233-6, 1998.

Levinson et al., Nature, 295:79, 1982.

Lin and Avery, Nature, 402:128-129, 1999.

Lin et al., Mol. Cell. Biol., 10:850, 1990.

Liu et al. Am J Pathol, 158(6):2057-66, 2001.

Lock et al., Nature Medicine 5:500-508, 2002.

Lock et al., Nature Medicine 8:500-508, 2002.

Lopez-Moratalla et al. Nitric Oxide, 1(1):95-104, 1997.

Lucotte et al., Mult. Scler. 6:78, 2000.

Luomala et al., Neurology 54:1862, 2000.

Luria et al., EMBO J., 6:3307, 1987.

Lusky and Botchan, Proc. Nat'l Acad. Sci. USA, 83:3609, 1986.

Lusky et al., Mol. Cell. Biol. 3:1108, 1983.

Macejak and Sarnow, Nature, 353:90-94, 1991.

Majors and Varmus, Proc. Nat'l Acad. Sci. USA, 80:5866, 1983.

Maniatis et al., Molecular Cloning, A Laboratory Manual, Cold Spring Harbor Press, Cold Spring Harbor, N.Y., 1989.

Mann et al., Cell, 33:153-159, 1983.

Mann et al., Neurology 54:552, 2000.

McLaughlin et al., J. Virol., 62(6):1963-1973, 1988.

McNeall et al., Gene, 76:81, 1989.

Miksicek et al., Cell, 46:203, 1986.

Miller et al., Am. J. Clin. Oncol., 15(3):216-221, 1992.

Miterski et al., Genes Immun. 1:37, 1999.

Montgomery et al., Proc. Nat'l Acad. Sci. USA, 95:155-2-15507, 1998.

Mordacq and Linzer, Genes and Dev., 3:760, 1989.

Moreau et al., Nucl. Acids Res., 9:6047, 1981.

Mueller and Wold, Science 246, 780-786, 1989.

Muesing et al., Cell, 48:691, 1987.

Muller-Ladner et al., J Neurol. Sci., 144(1-2):135-41, 1996.

Murga et al., Immunity 15; 959-970, 2001.

Muzyczka, Curr Top Microbiol Immunol, 158:97-129, 1992.

Mycko et al., Ann. Neurol. 44:70, 1998.

Mycko et al., J. Neuroimmunol., 84:198, 1998.

Myhr et al., Neurology 52:1771, 1999.

Nabel and Baltimore, Nature 326:711-713, 1987.

Nakamura et al., In: Handbook of Experimental Immunology (4^{th} Ed.), Weir et al., (eds). 1:27, Blackwell Scientific Publ., Oxford, 1987.

Naldini et al., Science, 272(5259):263-267, 1996.

Ng et al., Nuc. Acids Res., 17:601, 1989.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Niino et al., J Neurol. Sci. 179:70, 2000.

Nomoto et al., Gene, 236(2):259-271, 1999.

Ohara et al., Proc. Nat'l Acad. Sci. USA, 86: 5673-5677, 1989.

Omirulleh et al., Plant Mol. Biol., 21:415-28, 1993.

Ondek et al., EMBO J., 6:1017, 1987.

Omitz et al., Mol. Cell. Biol., 7:3466, 1987.

Padberg et al., J. Neuroimmunol., 99(2):218-23, 1999.

Palmiter et al., Nature, 300:611, 1982.

Paskind et al., Virology, 67:242-248, 1975.

PCT Application No. 95/06128

PCT Application No. WO 94/09699

PCT Application No. WO 94/09699

PCT Application PCT/US87/00880

PCT Application PCT/US89/01025

PCT Application WO 88/10315

PCT Application WO 90/07641

PCT Application WO89/06700

Pech et al., Mol. Cell. Biol., 9:396, 1989.

Pelletier and Sonenberg, Nature, 334:320-325, 1988.

Perales et al., Proc. Nat'l Acad. Sci. USA, 91:4086-4090, 1994.

Perez-Stable and Constantini, Mol. Cell. Biol., 10:1116, 1990.

Picard and Schaffner, Nature, 307:83, 1984.

Pinkert et al., Genes and Dev., 1:268, 1987.

Plasterk and Ketting, Curr. Opin. Genet. Dev., 10:562-567, 2000.

Ponta et al., Proc. Nat'l Acad. Sci. USA, 82:1020, 1985.

Porton et al., Mol. Cell. Biol., 10:1076, 1990.

Potrykus et al., Mol. Gen. Genet., 199:183-188, 1985.

Potter et al., Proc. Nat'l Acad. Sci. USA, 81:7161-7165, 1984.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Ren et al., Genes Dev., 16: 245-256, 2002.

Resendez Jr. et al., Mol. Cell. Biol., 8:4579, 1988.

Rice, Mathematical Statistics and Data Analysis, Duxbury Press, Belmont, CA, 1995.

Rickert, Oncogene 12: 2631-40, 1996.

Ridgeway, In: Vectors: A survey of molecular cloning vectors and their uses, Stoneham: Butterworth, pp. 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rosen et al., Cell, 41:813, 1988.

Roux et al., Proc. Nat'l Acad. Sci. USA, 86:9079-9083, 1989.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Sarchielli et al., J. Neuroimmunol., 80(1-2):76-86, 1997.

Satake et al., J. Virology, 62:970, 1988.

Schaffner et al., J. Mol. Biol., 201:81, 1988.

Searle et al., Mol. Cell. Biol., 5:1480, 1985.

Sharp and Marciniak, Cell, 59:229, 1989.

Sharp, Genes Dev., 13:139-141, 1999.

Sharpand Zamore, Science, 287:2431-2433, 2000.

Shaul and Ben-Levy, EMBO J, 6:1913, 1987.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J Virology, 62:427, 1988.

Spandidos and Wilkie, EMBO J, 2:1193, 1983.

Statistical Toolbox, Math Works, Inc., Version 2.1.1, 1997.

Stelmasiak et al., Med. Sci. Monit., 7(5): 914-8, 2001.

Stephens and Hentschel, Biochem. J., 248:1, 1987.

Storey and Tibshirani, Stanford tech report, 2001.

Streit et al, J. Invest. Dermatol., 105(4):562-6, 1995.

Stuart et al., Nature, 317:828, 1985.

Sullivan and Peterlin, Mol. Cell. Biol., 7:3315, 1987.

Svenningsson et al., Neurology, 53(8):1880-2, 1999.

Swartzendruber and Lehman, J. Cell. Physiology, 85:179, 1975.

Tabara et al., Cell, 99:123-132, 1999.

Takebe et al., Mol. Cell. Biol., 8:466, 1988.

Takiguchi et al., Am. J. Physiol., 274(2 Pt 1):E265-70, 1998.

Tavernier et al., Nature, 301:634, 1983.

Taylor and Kingston, Mol. Cell. Biol., 10: 165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al., J. Virology, 62:614, 1988.

Thomas, Genome Research 11, 1227-1236, 2001.

Tienari et al., Lancet 340:987-91, 1992.

Tratschin et al., Mol. Cell. Biol., 4:2072-2081, 1984.

Treisman, Cell, 42:889, 1985.

Tronche et al., Mol. Biol. Med., 7:173, 1990.

Trudel and Constantini, Genes and Dev. 6:954, 1987.

Tsumaki et al., J. Biol. Chem. 273(36):22861-22864, 1998.

Tur-Kaspa et al., Mol. Cell Biol., 6:716-718, 1986.

Tusher et al., Proc. Nat'l Acad. Sci. USA 98, 5116-5121, 2001.

Two tailed t-test: www.psycho.uni-duesseldorf.de/aap/proj ects/gpower/reference/

Tyndell et al., Nuc. Acids. Res., 9:6231, 1981.

Vandenbroeck et al., Genes Immunol 1:460, 2000.

Vandenbroeck et al., J. Neuroimmunol. 76:189, 1997.

Vannice and Levinson, J. Virology, 62:1305, 1988.

Vasseur et al., Proc Nat'l Acad. Sci. U.S.A., 77:1068, 1980.

Wagner et al., Proc. Nat'l Acad. Sci. USA 87(9):3410-3414, 1990.

Wagner et al., Science, 260:1510-1513, 1993.

Walker et al., Proc. Nat'l Acad. Sci. USA, 89:392-396 1992.

Walter et al., J. Clin. Invest. 87:1266, 1991.

Wang and Calame, Cell, 47:241, 1986.

Warrington et al., Physiol. Genomics 2: 143-7, 2000.

Weber et al., Cell, 36:983, 1984.

Weinberger et al., Mol. Cell. Biol., 8:988, 1984.

Wilson et al., Science, 244:1344-1346, 1989.

Winer et al., J. Immunol., 166(7):4751-6, 2001.

Winoto and Baltimore, Cell 59:649, 1989.

Wong et al., Gene, 10:87-94, 1980.

Wu and Wu, J. Biol. Chem., 262:4429-4432, 1987.

Wu and Wu, Adv. Drug Delivery Rev., 12:159-167, 1993.

Wu and Wu, Biochemistry, 27:887-892, 1988.

Wu et al., Nature 414(6862):457-62, 2001.

Wu et al, Biochem Biophys Res Commun., 233(1):221-226, 1997.

Yamashita et al, J Neurol. Sci., 153(1):32-4, 1997.

Yang et al., Proc. Nat'l Acad. Sci. USA, 87:9568-9572, 1990.

Yuceyar et al., Clin. Neurol. Neurosurg., 103(4):206-11, 2001.

Yutzey et al., Mol. Cell Biol., 9:1397, 1989.

Zhao-Emonet et al, Biochem. Biophys. Acta., 1442(2-3):109-19, 1998.

Zufferey et al, Nat. Biotechnol., 15(9):871-875, 1997.

What is claimed is:

1. A method of predicting whether a subject is or will be afflicted with multiple sclerosis (MS) comprising:
(a) obtaining an mRNA-containing sample from said subject;
(b) determining expression information for one or more genes from the group consisting of phosphatidylinositol transfer protein, inducible nitric oxide synthase, CIC-1 muscle chloride channel protein, placental bikunin, receptor kinase ligand LERK-3, GATA-4, thymopoietin, transcription factor E2f-2, S-adenosylmethionine synthetase, carcinoembryonic antigen, ret transforming gene, G protein-linked receptor, GTP-binding protein RALB, tyrosine kinase Syk, T cell leukemia LERK-2, tyrosine kinase (ELK1) oncogene, transcription factor SL1, phospholipase c, gastricsin, and D13S824E locus;
(c) comparing expression information for said selected genes with the expression information of the same genes in a subject not afflicted with MS; and
(d) predicting whether said subject is or will be afflicted with MS.

2. The method of claim 1, wherein the expression information for more one gene in the group of claim 1(b) is determined.

3. The method of claim 1, wherein the expression information for at least 2 genes in the group of claim 1(b) is determined.

4. The method of claim 1, wherein the expression information for at least 3 genes in the group of claim 1(b) is determined.

5. The method of claim 1, wherein the expression information for at least 4 genes in the group of claim 1(b) is determined.

6. The method of claim 1, wherein the expression information for at least 5 genes in the group of claim 1(b) is determined.

7. The method of claim 1, wherein the expression information for at least 6 genes in the group of claim 1(b) is determined.

8. The method of claim 1, wherein the expression information for at least 7 genes in the group of claim 1(b) is determined.

9. The method of claim 1, wherein the expression information for at least 8 genes in the group of claim 1(b) is determined.

10. The method of claim 1, wherein the expression information for at least 9 genes in the group of claim 1 (b) is determined.

11. The method of claim 1, wherein the expression information for 10-20 genes in the group of claim 1(b) is determined.

12. The method of claim 1, wherein said sample comprises peripheral blood-derived mononuclear cells.

13. The method of claim 1, further comprising determining expression information for an additional one or more genes in Tables 1, 4, 7, and 10.

14. The method of claim 1, further comprising determining expression information for one or more genes in Tables 2, 5, 8, and 11.

15. The method of claim 1, further comprising determining expression information for one or more genes in Tables 3, 6, 9, and 12.

16. The method of claim 1, further comprising obtaining expression information for each gene in step (b) from a subject not afflicted with MS.

17. The method of claim 1, wherein expression information is determined by microarray analysis of mRNA transcripts.

18. The method of claim 1, wherein expression information is determined by multiplex PCR of transcripts, northern blot, quantitative real time PCR, reverse trancription PCR (RT-PCR), or RNAse protection.

19. The method of claim 1, wherein expression information is determined by immunohistochemistry, ELISA or western blot.

20. The method of claim 17, wherein microarray analysis comprises use of oligonucleotides that hybridize to mRNA transcripts or cDNAs for the selected genes, and wherein the oligonucleotides are disposed or directly synthesized on the surface of a chip or wafer.

21. The method of claim 20, wherein said oligonucleotides are about 10 to about 50 base pairs in length.

22. A chip or wafer comprising a nucleic acid microarray, wherein said nucleic acids hybridize to target transcripts or cDNAs for phosphatidylinositol transfer protein, inducible nitric oxide synthase, CIC-1 muscle chloride channel protein, placental bikunin, receptor kinase ligand LERK-3, GATA-4, thymopoietin, transcription factor E2f-2, S-adenosylmethionine synthetase, carcinoembryonic antigen, ret transforming gene, G protein-linked receptor, GTP-binding protein RALB, tyrosine kinase Syk, T cell leukemia LERK-2, tyrosine kinase (ELK1) oncogene, transcription factor SL1, phospholipase c, gastricsin, and D13S824E locus.

23. The chip of claim 22, wherein said chip is comprised of polymers, plastics, resins, polysaccharides, silica or silica-based materials, fiberoptic materials, carbon, metals, inorganic glasses, or nitrocellulose.

24. The chip of claim 22, wherein said nucleic acids are cDNAs.

25. The chip of claim 22, wherein said nucleic acids are oligonucleotides.

26. The chip of claim 22, wherein said oligonucleotides are about 10 to about 50 base pairs or less in length.

27. A method for monitoring a therapy for multiple sclerosis comprising:
(a) obtaining an mRNA-containing sample from a subject receiving said therapy;
(b) determining expression information for one or more genes comprising phospatidylinositol transfer protein, inducible nitric oxide synthase, CIC-1 muscle chloride channel protein, placental bikunin, receptor kinase ligand LERK-3, GATA-4, thymopoietin, transcription factor E2f-2, S-adenosylmethionine synthetase, carcinoembryonic antigen, ret transforming gene, G protein-linked receptor, GTP-binding protein RALB, tyrosine kinase Syk, T cell leukemia LERK-2, tyrosine kinase (ELK1) oncogene, transcription factor SL1, phospholipase c, gastricsin, and D13S824E locus; and
(c) comparing expression information for said selected genes with the expression information of the same genes in an MS subject not receiving said therapy.

28. The method of claim 27, further comprising determining expression information for an additional one or more genes in Tables 1, 4, 7, and 10.

29. The method of claim 27, further comprising determining expression information for one or more genes in Tables 2, 5, 8, and 11.

30. The method of claim 27, further comprising determining expression information for one or more genes in Tables 3, 6, 9, and 12.

31. The method of claim 27, wherein said sample is peripheral blood.

32. The method of claim 27, further comprising modifying said therapy based upon the altered expression of one or more of said selected genes.

33. The method of claim 27, further comprising making a prediction on the efficacy of treating the subject from which said sample was obtained.

34. The method of claim 27, wherein the expression information is determined by microarray analysis of transcripts.

35. The method of claim 27, wherein the expression information is determined by multiplex PCR of transcripts, northern blot, quantitative real time PCR, reverse trancription PCR (RT-PCR), or RNAse protection.

36. The method of claim 27, wherein the expression information is determined by immunohistochemistry, western blot or ELISA.

37. The method of claim 34, wherein microarray analysis comprises use of oligonucleotides that hybridize to transcripts or cDNAs for the selected genes, and wherein the oligonucleotides are disposed or synthesized directly on the surface of a chip or wafer.

38. The method of claim 37, wherein said chip is comprised of polymers, plastics, resins, polysaccharides, silica or silica-based materials, carbon, metals, inorganic glasses, or nitrocellulose.

39. The method of claim 37, wherein said oligonucleotides are about 10 to about 50 base pairs or less in length.

40. The method of claim 27, wherein said MS subject not receiving said therapy is the same subject prior to receiving said therapy.

41. The method of claim 27, further comprising determining expression information for said selected genes from said subject at multiple time points.

42. A method for determining the efficacy of a therapy for multiple sclerosis comprising:
(a) obtaining an mRNA-containing sample from a subject receiving said therapy;
(b) determining expression information for one or more selected genes selected from the group consisting of skeletal muscle LIM-prot SLIM1, R kappa B, 815A9.1 myosin heavy chain, γ G2 psi from γ crystallin, thrombospondin 4, KIAA0178 (or Z97054);
(c) comparing expression information for said one or more selected genes with the expression information of the same gene or genes in an MS subject not receiving said therapy; and
(d) determining the efficacy of said therapy based on the ability of said therapy to alter the expression of said one or more genes.

43. The method of claim 42, further comprising determining expression information for one or more genes in Table 14.

44. The method of claim 43, further comprising determining expression information for one or more genes in Table 15.

45. A method for treating multiple sclerosis (MS) comprising administering to a subject with MS a drug that causes an increase in the level of a gene product selected from the group consisting of those genes indicated by a minus (−) sign in Tables 1-12.

46. A method for treating multiple sclerosis (MS) comprising administering to a subject with MS a drug that causes a decrease in the level of a gene product selected from the group consisting of those genes indicated by a plus (+) sign in Tables 1-12 and 16.
